SEQUENCE LISTING (57 sequences)

SEQ ID NO: 1 - DNA, 83 bp, Artificial
  misc_feature (23)..(62): n is any of A, G, T and C
gtggaaccgacagtggtacgtgnnnnnnnnnnnnnnnnnnnnnnnnnnnnnnnnnnnnnnnncacgagtgaagtctgagctcc

SEQ ID NO: 2 - DNA, 83 bp, Artificial; identical to SEQ ID NO: 1
  misc_feature: chemically synthesized

SEQ ID NO: 3 - DNA, 20 bp, Artificial
  misc_feature: Primer
gtggaaccgacagtggtacg

SEQ ID NO: 4 - DNA, 20 bp, Artificial
  misc_feature: Primer
gtggaaccgacagtggtacg

SEQ ID NO: 5 - DNA, 38 bp, Artificial
  misc_feature: Primer
tctaatacgactcactataggagctcagacttcactcg

SEQ ID NO: 6 - DNA, 15 bp, Artificial
  misc_feature: Primer
accgacagtggtacg

SEQ ID NO: 7 - DNA, 35 bp, Artificial
  misc_feature: Primer
gtcctaccgtcagatgtggaaccgacagtggtacg

SEQ ID NO: 8 - DNA, 74 bp, Artificial
  misc_feature: chemically synthesized
acagtggtacgtgnnnnnnnnnnnnnnnnnnnnnnnnnnnnnnnnnnnnnnnncacgagtgaagtctgagctcc

SEQ ID NO: 9 - DNA, 87 bp, Artificial
  misc_feature: chemically synthesized
tgatgtggaaccgacagtggtacgtgnnnnnnnnnnnnnnnnnnnnnnnnnnnnnnnnnnnnnnnncacgagtgaagtctgagctcc

SEQ ID NO: 10 - DNA, 26 bp, Artificial
  misc_feature: Primer
tgatgtggaaccgacagtggtacgtg

SEQ ID NO: 11 - DNA, 38 bp, Artificial; identical to SEQ ID NO: 5
  misc_feature: Primer

SEQ ID NO: 12 - DNA, 13 bp, Artificial
  misc_feature: Primer
ugauguggaaccg

SEQ ID NO: 13 - DNA, 13 bp, Artificial
  misc_feature: Primer
tgatguggaaccg

SEQ ID NO: 14 - DNA, 83 bp, Artificial; identical to SEQ ID NO: 1
  misc_feature: chemically synthesized

SEQ ID NO: 15 - DNA, 83 bp, Artificial; identical to SEQ ID NO: 1
  misc_feature: chemically synthesized

SEQ ID NO: 16 - DNA, 40 bp, Artificial
  misc_feature: Primer
aattgtcctactcgtcagatgtggaaccgacagtggtacg

SEQ ID NO: 17 - DNA, 38 bp, Artificial; identical to SEQ ID NO: 5
  misc_feature: Primer

SEQ ID NO: 18 - DNA, 21 bp, Artificial
  misc_feature: Primer
aattgtcctactcgtcagatg

SEQ ID NO: 19 - DNA, 21 bp, Artificial
  misc_feature: Primer
aattgtcctactcgtcagatg

SEQ ID NO: 20 - DNA, 135 bp, Artificial
  misc_feature: chemically synthesized
gcgagttcctctcagcgtgggannnnnnnnnnnnnnnnnnnnnnnnnnnnnnnnnnnnnnnngtcctatagtgagtcgtattagtagtcgcggagagtcgcaccctcaggatatcactcagcataatcatcagcg

SEQ ID NO: 21 - DNA, 48 bp, Artificial
  misc_feature: chemically synthesized
gggannnnnnnnnnnnnnnnnnnnnnnnnnnnnnnnnnnnnnnngtcc

SEQ ID NO: 22 - DNA, 18 bp, Artificial
  misc_feature: Primer
gcgagttcctctcagcgt

SEQ ID NO: 23 - DNA, 29 bp, Artificial
  misc_feature: Primer
gcgactactaatacgactcactataggac

SEQ ID NO: 24 - DNA, 15 bp, Artificial
  misc_feature: Primer
tcccacgctgagagg

SEQ ID NO: 25 - DNA, 24 bp, Artificial
  misc_feature: Primer
tatagtgagtcgtattagtagtcg

SEQ ID NO: 26 - DNA, 18 bp, Artificial
  misc_feature: Primer
gcgagtucctctcagcgu

SEQ ID NO: 27 - DNA, 18 bp, Artificial
  misc_feature: Primer
gcgaguuccucucagcgu

SEQ ID NO: 28 - DNA, 83 bp, Artificial; identical to SEQ ID NO: 1
  misc_feature: chemically synthesized

SEQ ID NO: 29 - DNA, 83 bp, Artificial; identical to SEQ ID NO: 1
  misc_feature: chemically synthesized

SEQ ID NO: 30 - DNA, 20 bp, Artificial
  misc_feature: Primer
gtggaaccgacagtggtacg

SEQ ID NO: 31 - DNA, 20 bp, Artificial
  misc_feature: Primer
gtggaaccgacagtggtacg

SEQ ID NO: 32 - DNA, 19 bp, Artificial
  misc_feature: Primer
ggagctcagacttcactcg

SEQ ID NO: 33 - DNA, 74 bp, Artificial; identical to SEQ ID NO: 8
  misc_feature: chemically synthesized

SEQ ID NO: 34 - DNA, 87 bp, Artificial; identical to SEQ ID NO: 9
  misc_feature: chemically synthesized

SEQ ID NO: 35 - DNA, 26 bp, Artificial
  misc_feature: Primer
tgatgtggaaccgacagtggtacgtg

SEQ ID NO: 36 - DNA, 19 bp, Artificial
  misc_feature: Primer
ggagctcagacttcactcg

SEQ ID NO: 37 - DNA, 13 bp, Artificial
  misc_feature: Primer
ugauguggaaccg

SEQ ID NO: 38 - DNA, 13 bp, Artificial
  misc_feature: Primer
tgatguggaaccg

SEQ ID NO: 39 - DNA, 135 bp, Artificial; identical to SEQ ID NO: 20
  misc_feature: chemically synthesized

SEQ ID NO: 40 - DNA, 48 bp, Artificial; identical to SEQ ID NO: 21
  misc_feature: chemically synthesized

SEQ ID NO: 41 - DNA, 18 bp, Artificial
  misc_feature: Primer
gcgagttcctctcagcgt

SEQ ID NO: 42 - DNA, 29 bp, Artificial
  misc_feature: Primer
gcgactactaauacgactcactatnggac

SEQ ID NO: 43 - DNA, 15 bp, Artificial
  misc_feature: Primer
tcccacgctgagagg

SEQ ID NO: 44 - DNA, 25 bp, Artificial
  misc_feature: Primer
tatagtgagtcgtattagtagtcgc

SEQ ID NO: 45 - DNA, 18 bp, Artificial
  misc_feature: Primer
gcgaguuccucucagcgu

SEQ ID NO: 46 - DNA, 18 bp, Artificial
  misc_feature: Primer
gcgagtucctctcagcgu

SEQ ID NO: 47 - DNA, 83 bp, Artificial; identical to SEQ ID NO: 1
  misc_feature: chemically synthesized

SEQ ID NO: 48 - DNA, 83 bp, Artificial; identical to SEQ ID NO: 1
  misc_feature: chemically synthesized

SEQ ID NO: 49 - DNA, 20 bp, Artificial
  misc_feature: Primer
gtggaaccgacagtggtacg

SEQ ID NO: 50 - DNA, 19 bp, Artificial
  misc_feature: Primer
ggagctcagacttcactcg

SEQ ID NO: 51 - DNA, 20 bp, Artificial
  misc_feature: Primer
gtggaaccgacagtggtacg

SEQ ID NO: 52 - DNA, 19 bp, Artificial
  misc_feature: Primer
ggagctcagacttcactcg

SEQ ID NO: 53 - DNA, 83 bp, Artificial; identical to SEQ ID NO: 1
  misc_feature: chemically synthesized

SEQ ID NO: 54 - DNA, 83 bp, Artificial; identical to SEQ ID NO: 1
  misc_feature: chemically synthesized

SEQ ID NO: 55 - DNA, 20 bp, Artificial
  misc_feature: Primer
gtggaaccgacagtggtacg

SEQ ID NO: 56 - DNA, 38 bp, Artificial; identical to SEQ ID NO: 5
  misc_feature: Primer

SEQ ID NO: 57 - DNA, 61 bp, Artificial
  misc_feature: chemically synthesized
nnnnnnnnnnnnnnnnnnnnnnnnnnnnnnnnnnnnnnnncacgagtgaagtctgagctcc

(19) United States
(12) Patent Application Publication
(10) Pub. No.: US 2008/0070238 A1
(43) Pub. Date: Mar. 20, 2008

(54) Method for the Synthesizing Nucleic Acids, and Application Thereof

(76) Inventors: Sven Klussmann, Berlin (DE); Florian Jarosch, Berlin (DE); Axel Vater, Berlin (DE)

Correspondence Address: BELL, BOYD, & LLOYD LLP, P.O. BOX 1135, CHICAGO, IL, 60690, US

(21) Appl. No.: 10/572,291

(22) PCT Filed: Sep. 14, 2004
(86) PCT No.: PCT/EP04/10268

§ 371 Date: Feb. 23, 2007

(30) Foreign Application Priority Data
Sep. 14, 2003 (DE) 103 42 373.7

(51) Int. Cl. C12Q 1/68; C12P 19/34

(52) U.S. Cl. 435/6; 435/91.1

(57) ABSTRACT

The invention relates to a method for synthesizing a nucleic acid comprising modified nucleotides. Said method encompasses the following steps: a matrix strand is provided; —a primer which at least partially hybridizes on the matrix strand is provided; —nucleoside triphosphates, at least some of which are modified nucleoside triphosphates, are provided; —a polymerase activity is supplied; and —the matrix strand, the primer, and the nucleoside triphosphates are incubated so as to synthesize a nucleic acid that is substantially complementary to the matrix strand. The inventive method is characterized in that the polymerase activity represents a reverse transcriptase activity.

The present invention is related to a method for the synthesis of nucleic acids and its use in methods for the selection of target molecule binding nucleic acids.

### Functions of Oligonucleotides

Since the discovery of Cech and Altman who described for the first time that RNA is not only active in C. elegans as a messenger molecule between the genome, i.e. DNA, and the protein synthesis machinery, i.e. the ribosomes, but exhibits catalytic activities, numerous papers have been published in the field of ribozymes, i.e. catalytic RNA oligonucleotides and aptamers, i.e. target molecule binding (deoxy)oligonucleotides. Both approaches are suitable as such or in combination, for use as therapeutics, diagnostics, for target validation or as media for affinity chromatography or specific adsorbers.

A further approach to industrial application of oligonucleotides are antisense molecules as well as siRNAs which may result in post-transcriptional suppression of a specific gene expression.

### Stabilisation of Functional Oligonucleotides

A significant limitation is imposed on the use of oligonucleotides by their rapid degradation through ubiquitous RNases and DNases. Particularly in biological systems RNases and DNases can be found in significant amounts and result in lifetimes of RNA and DNA oligonucleotides of a few seconds to minutes (Griffin et al., 1993; Jellinek et al., 1995; Lin et al., 1994).

Methods to protect oligonucleotides from being attacked by exonucleases are in most cases based on the modification of their ends by the addition of protective groups such as, e.g., a terminal inverted nucleotide, i.e. 3′-3′ or 5′-5′ linkages or other large groups such as, for example, polyethylene glycol (Bell et al., 1999). The replacement of natural substituents protects similarly against exonucleases and endonucleases, particularly at the 2′ carbon of the ribose and at the phosphor. Non-natural, nuclease-resistant alternatives to ribose (2′-OH) or deoxyribose (2′-H) are: 2′-amino, 2′-fluoro, 2′-azido, 2′-O-methyl, 2′-alkyl, 2′-allyl and arabino nucleotides (Eaton and Pieken, 1995). The most frequent phosphor modification is the replacement of oxygen by sulfur which results in so-called phosphorothioates.

Measures of this kind provide for significantly longer liftetimes in biological liquids, which may be up to several hours (Eaton and Pieken, 1995; Green et al., 1995; Jellinek et al., 1995; Lin et al., 1994).

Any of the aforementioned modifications, however, goes along with some limitations. In particular the synthesis of modified oligonucleotides by DNA or RNA polymerases is in most cases not possible. This, however, is frequently a requirement for the identification of functional oligonucleotides. It is particularly by the immediate link between the phenotype (the structure and thus the function) and the genotype (the nucleic acid sequence) inherent to the oligonucleotides, and by their characteristic that they can be copied, that it is possible to enrich suitable nucleic acid sequences from natural libraries, as represented by the genome and the transcriptome, or from synthetic libraries, for example combinatorial libraries, by selection and amplification to such an extent that single sequences having the desired characteristics can be isolated.

Such modifications which cannot be reconciled with the enzymatic processes, can only be introduced by chemical synthesis of the identified sequences subsequent to the identification of the functional oligonucleotides. At first, it is necessary that the identified sequences can be chemically synthesized. Therefore, the candidates being RNA hybrids typically having a length of about 60 to 90 nucleotides, are first shortened to 50 nucleotides or less in order to allow for an efficient chemical synthesis. Subsequently, the unmodified purines are replaced by 2′-modified nucleotides. Frequently, the function of the oligonucleotide is thus affected (Kujau et al., 1997). Therefore, in only a minority of cases all nucleotides can be modified afterwards. Because of this, most of the stabilized oligonucleotides have some weak points in the molecule which may be subject to an attack by a nuclease, thereby reducing their lifetime.

### Current Enzymatic Methods for the Stabilization of Oligonucleotides

Phosphorothioate may be enzymatically introduced by RNA polymerases ((Jhaveri et al., 1998) or by Taq-DNA polymerases, whereby, however, it is only possible to incorporate up to three phosphorothioate DNTPs (King et al., 2002). Phosphorothioates are apart from that disadvantageous insofar as they are cytotoxic (Henry et al., 2001).

2′-fluoro and 2′-amino modifications can be introduced into RNA by transcription. A review on modified aptamers has been prepared by Kusser (Kusser, 2000). Also the enzymatic incorporation of 2′-O-methyl and 2′-azido nucleoside triphosphates using T7-RNA polymerase has been described (Lin et al., 1994; Padilla and Sousa, 1999; Padilla and Sousa, 2002). The incorporation of such modified nucleotides is, however, limited to modified cytidines and uridines for the time being (Aurup et al., 1992). In particular, 2′-modified guanosines are not tolerated by known RNA polymerases. The reason therefor seems to reside in the instability of the complex of polymerase and DNA during the initial phase of the RNA polymerization. This stage, comprising the polymerization of the first 8 to 12 nucleotides has to be performed as quickly as possible as otherwise the RNA polymerase rapidly leaves the complex again (Lin et al., 1994; Milligan and Uhlenbeck, 1989).

A prerequisite for a successful initiation of the RNA synthesis, however, is at least one guanosine at the first two positions to be transcribed. An optimum initiation sequence even requires three consecutive guanosines at positions 1 to 3 of the RNA for T7- and T3-RNA polymerase (Milligan and Uhlenbeck, 1989) and GAAGNG for the SP6-RNA polymerase, such as, for example, presented in Meador et al., 1995.

Therefore, it has factually been impossible to date to incorporate 2′-modified guanosine nucleotides into RNA molecules and to synthesize completely 2′-modified nucleic acids by means of RNA polymerases, respectively.

Therefore, the objective underlying the present invention was to provide for an enzymatic method which allowed the incorporation of modified nucleotides and in particular of 2′-fluoro modified nucleotides into a nucleic acid. In connection therewith it was a particular objective to provide a method allowing to incorporate for all of the five naturally occurring bases (A, C, G, T, U), base modifications thereof and possible universal bases such as, for example, inosine (I), as nucleoside phosphates in their sugar modified form into nucleic acids.

A further objective underlying the present invention was to provide for a method for enzymatic synthesis of nucleic acids, whereby the nucleic acids completely consist of modified nucleoside phosphates, in particular 2′-fluoro-modified nucleoside phosphates.

Finally, another objective underlying the present invention was to provide a method for the selection of nucleic acids binding to a target molecule, whereby the nucleic acids partially or completely consist of modified nucleoside phosphates and in particular 2′-fluoro-modified nucleoside phosphates.

The objective is solved in accordance with the present invention by the methods of the independent claims. Preferred embodiments may be taken from the subclaims.

The problem underlying the present invention is solved in a first aspect by a method for the synthesis of a nucleic acid, whereby the nucleic acid comprises modified nucleotides, comprising the steps of:
providing a template strand;
providing a primer which is at least partially hybridizing to the template strand;
providing nucleoside triphosphates, whereby a portion of the nucleoside triphosphates are modified nucleoside triphosphates;
providing a polymerase activity; and
incubating the template strand, the primers, the nucleoside triphosphates for the synthesis of a nucleic acid which is essentially complementary to the template strand,
characterized in that the polymerase activity is a reverse transcriptase activity.

In an embodiment the reverse transcriptase is selected from the group comprising reverse transcriptases of murine moloney leukemia virus (MMLV), avian myeloblastosis virus (AMV), thermostable reverse transcriptases, DNA polymerase of Carboxydothermus hydrogenoformans, respective mutants thereof, and mixtures thereof.

In an embodiment the modified nucleoside triphosphates are selected from the group comprising 2′-fluoro-modified nucleoside triphosphates, 2′-amino-modified nucleoside triphosphates, 2′-azido-modified nucleoside triphosphates, 2′-O-methyl-modified nucleoside triphosphates, 2′-alkyl-modified nucleoside triphosphates, 2′-allyl-modified nucleoside triphosphates, arabino-nucleoside triphosphates and nucleotide phosphorothioates.

In an embodiment the modified nucleoside triphosphates are 2′-fluoro nucleoside triphosphates.

In an embodiment the nucleoside triphosphates provided are exclusively modified nucleoside triphosphates and that the synthesized nucleic acid preferably essentially consists solely of modified nucleotides.

In an embodiment the template strand consists of RNA.

In an alternative embodiment the template strand consists of DNA.

In a further alternative embodiment the template strand consists of a modified nucleic acid, preferably a 2′-fluoro nucleic acid.

In an alternative the sequence of the primer is part of the nucleic acid to be synthesized.

In an embodiment the sequence of the primer is different from the nucleic acid to be synthesized.

In an embodiment the primer consists of modified nucleoside phosphates, whereby the modification of the nucleoside phosphates of the primer is the same modification as the one of the nucleoside triphosphates provided.

In an embodiment the primer consists of RNA.

In an embodiment the primer consists of DNA, whereby at least the 3′ terminal nucleotide of the primer is a deoxyribonucleotide.

In an embodiment the polymerase activity synthesizes a strand which is essentially complementary to the template strand, whereby it is preferably base paired with the template strand.

In a preferred embodiment the synthesized nucleic acid is separated from the template strand.

In an embodiment the primer or a part thereof is removed from the nucleic acid synthesized by the polymerase activity.

In an embodiment the template strand and/or the primer is digested or cleaved, preferably after the synthesis of the nucleic acid which is essentially complementary to the template strand.

In a preferred embodiment the separation and/or the cleavage is performed by alkaline cleavage or enzymatic activity.

The problem underlying the present invention is solved in a second aspect by the use of a reveres transcriptase for the synthesis of a nucleic acid, whereby the nucleic acid comprises at least a modified nucleoside phosphate.

In an embodiment according to the second aspect of the present invention the reverse transcriptase is selected from the group comprising reverse transcriptases of murine moloney leukemia virus (MMLV), avian myeloblastosis virus (AMV), thermostable reverse transcriptases, DNA polymerase of Carboxydothermus hydrogenoformans, mutants thereof, and mixtures thereof.

In an embodiment the modified nucleoside triphosphate is selected from the group comprising 2′-fluoro-modified nucleoside triphosphates, 2′-amino-modified nucleoside triphosphates, 2′-azido-modified nucleoside triphosphates, 2′-O-methyl-modified nucleoside triphosphates, 2′-alkyl-modified nucleoside triphosphates, 2′-allyl-modified nucleoside triphosphates, arabino nucleoside triphosphates and nucleoside phosphorothioates.

In an embodiment the nucleic acid essentially consists completely of modified nucleoside phosphates.

The problem underlying the present invention is solved in a third aspect by a method for the selection of a target molecule binding nucleic acid, in particular of aptamers, comprising the steps of:
(a) providing a heterogeneous population of nucleic acids, in particular D-nucleic acids, whereby any of the nucleic acids comprises a region having a randomized sequence and a first constant sequence at the 5′ end and a second constant sequence at the 3′ end, and whereby the nucleic acids forming the population differ in the randomized sequence,
(b) contacting the population of nucleic acids with the target molecule,
(c) separating the nucleic acids not interacting with the target molecule,
(d) separating from the target molecule the nucleic acid(s) interacting with the target molecule,
(e) optionally repeating the steps (a) to (d), whereby the nucleic acid(s) from step (d) form the heterogeneous population or is/are contained therein,
(f) reverse transcription of the nucleic acid(s) interacting with the target molecule, in order to form reverse transcription products,
(g) performing a second strand synthesis, whereby the second strand is essentially complementary to the reverse transcription products, whereby the second strand synthesis is preferably an amplification reaction and preferably a polymerase chain reaction,
(h) transcription of the product of (g), whereby the synthesized second strand serves as a template strand, to obtain transcription products,
(i) synthesis of the nucleic acids which are essentially complementary to the transcription products,
(j) optionally repeating steps (a) to (i), whereby the nucleic acid(s) of step (i) form the heterogeneous population or are contained therein, and
(k) optionally sequencing the nucleic acid(s) obtained from step (f) or (g),
characterized in that the synthesis according to step (i) is performed in accordance with a method according to the first or the second aspect of the present invention.

The problem underlying the present invention is solved in a fourth aspect by a method for the selection of a target molecule binding nucleic acid, in particular of aptamers, comprising the steps of:
(a) providing a heterogeneous population of nucleic acids, in particular D-nucleic acids, whereby any of the nucleic acids comprises a region having a randomized sequence and a first constant sequence at the 5′ end and a second constant sequence at the 3′ end, and whereby the nucleic acids forming the population differ in the randomized sequence,
(b) contacting the population of nucleic acids with the target molecule,
(c) separating the nucleic acids not interacting with the target molecule,
(d) separating from the target molecule the nucleic acid(s) interacting with the target molecule,
(e) optionally repeating steps (a) to (d), whereby the nucleic acid(s) of step (d) form the heterogeneous population or are contained therein,
(f) reverse transcription of the nucleic acid(s) interacting with the target molecule, in order to form reverse transcription products,
(g) performing a second strand synthesis, whereby the second strand is essentially complementary to the reverse transcription products, whereby the second strand synthesis is preferably an amplification reaction and preferably a polymerase chain reaction,
(h) transcription of the product of step (g), whereby the synthesized second strand serves as a template strand in order to obtain transcription products,
(i) synthesis of nucleic acids which are essentially complementary to the transcription products,
(j) optionally repeating steps (a) to (i), whereby the nucleic acid(s) of step (i) form the hydrogenous population or are contained therein, and
(k) optionally sequencing the nucleic acid(s) obtained from step (f) or (g),
characterized in that at least the randomized region of the nucleic acid and/or of the nucleic acid synthesized in step (i) essentially consists completely of modified nucleoside phosphates, preferably 2′-fluoro nucleoside phosphates.

The problem underlying the present invention is solved in a fifth aspect by a method for the selection of a target molecule binding nucleic acid, in particular of aptamers, in particular a method according to the third and the fourth aspect of the present invention, comprising the steps of:
(a) providing a heterogeneous population of nucleic acids, in particular D-nucleic acids, whereby any of the nucleic acids comprises a region having a randomized sequence and a first constant sequence at the 5′ end and a second constant sequence at the 3′ end and whereby the nucleic acids forming the population differ in the randomized sequence,
(b) contacting the population of nucleic acids with the target molecule,
(c) separating the nucleic acids not interacting with the target molecule,
(d) separating from the target molecule the nucleic acid(s) interacting with the target molecule,
(e) optionally repeating steps (a) to (d), whereby the nucleic acid(s) of step (d) form the heterogeneous population or are contained therein,
(f) reverse transcription of the nucleic acid(s) interacting with the target molecule, in order to form reverse transcription products,
(g) performing a second strand synthesis, whereby the second strand is essentially complementary to the reverse transcription products, whereby the second strand synthesis is preferably an amplification reaction and preferably a polymerase chain reaction,
(h) transcription of the product of step (g), whereby the synthesized second strand serves as a template strand in order to obtain transcription products,
(i) synthesis of nucleic acids which are essentially complementary to the transcription products,
(j) optionally repeating steps (a) to (i), whereby the nucleic acid(s) of step (i) form the hydrogenous population or are contained therein, and
(k) optionally sequencing the nucleic acid(s) obtained from step (f) or (g),
characterized in that
   the first constant sequence of the nucleic acid in step (a) comprises a forward primer sequence and the second constant sequence comprises a reverse primer binding site, and
   a reverse primer is used in the reverse transcription according to step (f) which is essentially complementary to the reverse primer binding site and comprises at its 5′ end a further partial region, and the reverse transcription product comprises in 5′→3′ direction a reverse primer sequence, a sequence essentially complementary to the randomized sequence and a forward primer binding site.

In an embodiment of the fifth aspect of the present invention the reverse primer and a forward primer are used in the second strand synthesis, whereby the forward primer is at least partially complementary to a part of the forward primer binding site of the reverse transcription product, whereby the sequence of the synthesized second strand is essentially identical to the sequence of the nucleic acid of step (d) and additionally comprises at the 3′ end a sequence which is essentially complementary to the further partial region of the reverse primer.

In an embodiment of the fifth aspect of the present invention the further partial region of the reverse primer is a promoter sequence, whereby preferably the promoter sequence is selected from the group comprising promoter sequences of the T7-RNA polymerase, the T3-RNA polymerase and the SP6 polymerase.

In an embodiment of the fifth aspect of the present invention the strand synthesized in the second strand synthesis is used as a template strand in a transcription reaction, whereby the transcription product comprises in 3′→5′ direction the forward primer binding site, the complementary randomized sequence and the reverse primer sequence.

In a preferred embodiment of the fifth aspect of the present invention the transcription product is reacted with a reverse transcriptase together with a forward synthesis primer and modified nucleoside triphosphates, preferably 2′-fluoro nucleoside phosphates, whereby the forward synthesis primer hybridizes to the forward primer binding site in order to obtain a synthesis product, whereby the synthesis product comprises modified nucleoside phosphates, preferably 2′-fluoro nucleoside phosphates.

In a preferred embodiment of the fifth aspect of the present invention the forward synthesis primer consists of modified nucleoside triphosphates.

In an embodiment of the fifth aspect of the present invention the template strand is subjected to an alkaline treatment in order to obtain a single-stranded nucleic acid, whereby the nucleic acid comprises in 5′→3′ direction the forward primer sequence, the randomized region and the reverse primer binding site.

In a preferred embodiment of the fifth aspect of the present invention the forward primer comprises at its 5′ end a further partial region and that the synthesized second strand comprises at its 5′ end a sequence corresponding to the further partial region.

In a still more preferred embodiment of the fifth aspect of the present invention the strand synthesized in the second strand synthesis is subjected to a transcription reaction as a template strand, whereby the transcription product comprises in 3′→5′ direction the forward primer binding site including the sequence complementary to the further partial region of the forward primer, the complementary randomized region and the reverse primer sequence at its 5′ end, whereby the reverse primer sequence preferably lacks a sequence corresponding to the further partial region of the reverse primer.

In a further preferred embodiment of the fifth aspect of the present invention the transcription product is reacted with a reverse transcriptase together with a forward synthesis primer and modified nucleoside triphosphate, preferably 2′-fluoro nucleoside phosphates, whereby the forward synthesis primer is hybridized to the forward primer binding site, in order to obtain a synthesis product, whereby the synthesis product comprises modified nucleoside phosphates, preferably 2′-fluoro nucleoside phosphates.

In a preferred embodiment of the fifth aspect of the present invention the forward synthesis primer consists of ribonucleotides or of deoxyribonucleotides having at least one ribonucleotide at its 3′ end.

In a further preferred embodiment of the fifth aspect of the present invention the template strand is subjected to an alkaline cleavage and the forward synthesis primer is cleaved off, in order to obtain a single-stranded nucleic acid, whereby the nucleic acid comprises in 5′→3′ direction the forward primer sequence, the randomized region and the reverse primer binding site.

In an embodiment of the fifth aspect of the present invention the forward primer and the reverse primer consist of DNA.

The problem underlying the present invention is solved in a sixth aspect by a method for the selection of a target molecule binding nucleic acid, in particular of aptamers, comprising the steps of:
(a) providing a heterogeneous population of nucleic acids, in particular D-nucleic acids, whereby any of the nucleic acids comprises a region having a randomized sequence and a first constant sequence at the 5′ end and a second constant sequence at the 3′ end, and whereby the nucleic acids forming the population differ in the randomized sequence,
(b) contacting the population of nucleic acids with the target molecule,
(c) separating the nucleic acids not interacting with the target molecule,
(d) separating from the target molecule the nucleic acid(s) interacting with the target molecule,
(e) optionally repeating steps (a) to (d), whereby the nucleic acid(s) of step (d) form the heterogeneous population or are contained therein,
(f) reverse transcription of the nucleic acid(s) interacting with the target molecule, in order to form reverse transcription products,
(g) performing an amplification reaction with the reverse transcription products, whereby the amplification reaction is preferably a polymerase chain reaction, in order to obtain an amplified reverse transcription product,
(h) synthesis of nucleic acids which are essentially complementary to the reverse transcription products amplified in (g), in order to obtain a synthesis product,
(i) optionally repeating steps (a) to (h), whereby the nucleic acid(s) of step (h) form the heterogeneous population or is contained therein,
(j) optionally sequencing of the nucleic acid obtained in step (f) or (g),
characterized in that
   the first constant sequence of the nucleic acid in step (a) comprises a forward primer sequence and the second constant sequence comprises a reverse primer binding site, and
   a reverse primer is used in the reverse transcription of step (f) which is essentially complementary to the reverse primer binding site and whereby the reverse transcription product comprises in 5′→3′ direction a reverse primer sequence, a sequence essentially complementary to the randomized sequence and a forward primer binding site essentially complementary to the forward primer sequence.

In an embodiment of the sixth aspect of the present invention the reverse primer and a forward primer are used in the second strand synthesis, whereby the forward primer is essentially complementary to the forward primer binding site, whereby the sequence of the synthesized second strand is essentially identical to the nucleic acid to be amplified.

In an embodiment of the sixth aspect of the present invention the synthesis after step (g) the amplified reverse transcription product is reacted with a forward synthesis primer, modified nucleoside triphosphates, preferably 2′-fluoro nucleoside triphosphates, and a reverse transcriptase, whereby the forward synthesis primer hybridizes to the forward primer binding site, in order to obtain a synthesis product, whereby the synthesis product comprises modified nucleoside phosphates, preferably 2′-fluoro nucleoside phosphates.

In an embodiment of the sixth aspect of the present invention the forward synthesis primer consists of modified nucleoside triphosphates.

In an embodiment of the sixth aspect of the present invention the template strand is subjected to digestion, preferably an enzymatic digestion, in order to obtain a single-stranded nucleic acid, whereby the nucleic acid comprises in 5′→3′ direction the forward primer sequence, the randomized region and the reverse primer binding site.

In a preferred embodiment of the sixth aspect of the present invention the second strand synthesis, the reverser primer and a forward primer are used, whereby the forward primer is essentially complementary to the forward primer binding site and comprises at its 5′ end a further partial region, whereby the partial region preferably has a length of about 10 to 25 and more preferably a length of about 10 to 15 nucleotides, whereby the partial region preferably is a binding site or a part thereof, for a forward synthesis primer, and an extended reverse transcription product is obtained, whereby the extended reverse transcription product corresponds to the reverse transcription product, whereby the reverse transcription product is supplemented at its 3′ end by a sequence, whereby the sequence is complementary to the sequence of the further partial region of the forward primer.

In a further preferred embodiment of the sixth aspect of the present invention the synthesis after step (g) the amplified reverse transcription product is reacted with a forward synthesis primer, modified nucleoside triphosphates, preferably 2′-fluoro nucleoside triphosphates, and a reverse transcriptase, whereby the forward synthesis primer hybridizes to the binding site for the forward synthesis primer in order to obtain a synthesis product, whereby the synthesis product comprises modified nucleoside phosphates, preferably 2′-fluoro nucleoside phosphates.

In an even more preferred embodiment of the sixth aspect of the present invention the forward synthesis primer consists of ribonucleotides or of deoxyribonucleotides having at least one ribonucleotide at its 3′ end.

In a preferred embodiment of the sixth aspect of the present invention the template strand is subjected to a digestion, preferably an enzymatic digestion, in order to obtain a single-stranded nucleic acid, whereby the nucleic acid comprises in 5′→3′ direction the forward primer sequence, the randomized region and the reverse primer binding site.

In an embodiment of the sixth aspect of the present invention the forward primer and the reverse primer consist of DNA.

The problem underlying the present invention is solved in a seventh aspect by a method for the selection of a target molecule binding nucleic acid, particularly of aptamers, comprising the steps of:
(a) providing a heterogeneous population of nucleic acids, in particular D-nucleic acids, whereby any of the nucleic acids comprises a region having a randomized sequence and a first constant sequence at the 5′ end and a second constant sequence at the 3′ end and whereby the nucleic acids forming the population differ in the randomized sequence,
(b) contacting the population of nucleic acids with the target molecule,
(c) separating the nucleic acids not interacting with the target molecule,
(d) separating from the nucleic acid the nucleic acid(s) interacting with the nucleic acid,
(e) optionally repeating steps (a) to (d), whereby the nucleic acid(s) of step (d) form the heterogeneous population or are contained therein,
(f) amplifying the nucleic acid of step (a) comprising the step of:
   reacting the nucleic acid of step (e) with a reverse transcriptase, a reverse primer, a forward primer and nucleoside phosphates, preferably modified nucleoside phosphates and more preferably 2′-F-nucleoside phosphates,
   whereby the reverse primer is essentially complementary to the reverse primer binding site and hybridizes thereto and carries a label, whereby the label is mediating an interaction between the primer and the interaction partner, and
   whereby the forward primer is essentially identical to the forward primer sequence of the nucleic acid of step (a),
   in order to obtain a double-stranded amplification product, whereby one strand essentially corresponds to the nucleic acid of step (a) and a strand is complementary thereto, whereby the complementary strand carries the label,
(g) removing the complementary strand from the amplification product in order to obtain a nucleic acid corresponding essentially to the nucleic acid of step (a),
(h) optionally repeating steps (a) to (g), whereby the nucleic acid of step (g) forms the heterogeneous population or is contained therein,
(i) optionally sequencing the nucleic acid(s) obtained from step (d), (f) or (g), whereby in case of sequencing preferably the following additional steps are performed:
   (ia) reverse transcription using the reverse primer, whereby the reverse primer consists of DNA and does not carry any label,
   (ib) amplifying the reverse transcription product of step (ia) by performing a second strand synthesis for the amplification, whereby the reverse primer and the forward primer are used, and whereby the reverse primer does not have any label and the forward primer consists of DNA.

In an embodiment of the seventh aspect of the present invention the complementary strand in step (g) is separated by interaction between the label and the interaction partner.

In a preferred embodiment of the seventh aspect of the present invention the interaction partner is immobilized to a surface.

In an even more preferred embodiment of the seventh aspect of the present invention the amplification product is immobilized at the surface by the interaction between the label and the interaction partners.

In an embodiment of the seventh aspect of the present invention the two strands of the amplification product are separated from each other, whereby preferably the complementary strand remains immobilized.

In an embodiment of the seventh aspect of the present invention the label is selected from the group comprising biotin, digoxigenin and linker having reactive functional groups and whereby the reactive functional groups are preferably selected from the group comprising amino, carboxy, epoxy and thiol.

In an embodiment of the seventh aspect of the present invention the interaction partner is selected from the group comprising streptavidin, avidin, neutravidin und anti-digoxigenin antibodies and complementary functional groups, and whereby the reactive functional groups are preferably selected from the group comprising amino, carboxy, epoxy and thiol.

In an embodiment of the seventh aspect of the present invention the label is attached at the 5′ and of the reverse primer.

In an embodiment of the seventh aspect of the present invention the forward primer comprises modified nucleoside phosphates, in particular 3′-fluoro nucleoside phosphates.

In an embodiment of the seventh aspect of the present invention the reverse primer comprises deoxynucleoside phosphates.

The problem underlying the present invention is solved in an eight aspect by a method for the selection of a target molecule binding nucleic acid, in particular of aptamers, comprising
(a) providing a heterogeneous population of nucleic acids, in particular D-nucleic acids, whereby any of the nucleic acids comprises a region having a randomized sequence and a first constant sequence at the 5′ end and a second constant sequence at the 3′ end and whereby the nucleic acids forming the population differ in the randomized sequence, whereby the nucleic acid comprises modified nucleoside phosphates, preferably 2′-fluoro-modified nucleoside phosphates, and each of the constant sequences comprises 4 to 6 nucleotides,
(b) contacting the population of nucleic acids with the target molecule,
(c) separating the nucleic acids not interacting with the target molecule,
(d) separating from the target molecule the nucleic acid(s) interacting with the target molecule,
(e) optionally repeating steps (a) to (d), whereby the nucleic acid(s) of step (d) form the heterogeneous population or are contained therein,
(f) modifying the nucleic acid of step (a) or (d) by the following steps:
   (f0) 5′ phosphorylating the 5′ terminal nucleotide of the nucleic acid of step (a), preferably by using a kinase, under the proviso that the 5′ terminal nucleotide does not already have a phosphate group at the 5′ end,
   (fa) providing a first adapter molecule, whereby the first adapter molecule consists of a double-stranded nucleic acid of a first and a second nucleic acid strand and whereby the first nucleic acid strand and the second nucleic acid strand are independently a deoxyribonucleic acid, a ribonucleic acid or an FNA, and whereby the 5′ end of the second nucleic acid strand provides for an overhang, whereby the overhang is at least partially complementary to the first constant partial region of the nucleic acid of step (a) and/or (d) or a part thereof,
   (fb) providing a second adapter molecule, whereby the second adapter molecule consists of a double-stranded nucleic acid of a first and a second nucleic acid strand, whereby the first nucleic acid strand carries a 5′ phosphate and the first and the second nucleic acid strand are independent from each other a deoxyribonucleic acid, a ribonucleic acid or an FNA, and whereby the 3′ end of the second nucleic acid strand provides for an overhang which is at least partially complementary to the second constant partial sequence of the nucleic acid of step (a) and/or (d) or a part thereof,
   (fc) ligating the first nucleic acid strand of the first end of the second adapter molecule to the nucleic acid of step (a) and/or (d), in order to obtain a ligation product as a reaction product,
(g) reverse transcription of the ligation product by using the second strand of the second adapter molecule present in the ligation reaction as a primer, in order to obtain a reverse transcription product,
(h) performing a second strand synthesis, whereby the second strand is essentially complementary to the reverse transcription product, whereby the second strand synthesis is more preferably an amplification reaction and preferably a polymerase chain reaction,
(i) transcription of the product of (h), whereby the synthesized second strand serves as a template strand in order to obtain transcription products, whereby a transcription product is obtained which is complementary to
   the sequence of the first nucleic acid strand of the first adapter molecule,
   the first constant partial sequence,
   the randomized region, and
   the second constant partial sequence; and
(j) performing a nucleic acid synthesis, whereby the transcription product of step (i) is reacted with a forward synthesis primer, modified nucleoside triphosphates, preferably 2′-fluoro nucleoside triphosphates, and a reverse transcriptase, whereby the primer hybridizes to the complementary sequence of the first nucleic acid strand of the first adapter molecule, and whereby the primer consists of RNA or a combination of RNA and DNA, under the proviso that in case of a combination of RNA and DNA at least the 3′ end is formed by a ribonucleotide,
(k) cleaving off the transcription product after step (j) and the forward primer sequence of the nucleic acid molecule synthesized in step (j), in order to obtain a nucleic acid which is essentially identical to the nucleic acid of step (a) or (d),
(l) optionally repeating steps (a) to (k), whereby the nucleic acid of step (k) forms the heterogeneous population or is contained therein, and
(m) optionally sequencing the nucleic acid obtained in step (h).

In an embodiment of the eighth aspect of the present invention the cleavage in step (k) is an alkaline cleavage and/or is performed by RNAase digestion.

The problem underlying the present invention is solved in a ninth aspect by a method for the selection of a target molecule binding nucleic acid, in particular of aptamers, comprising
(a) providing a heterogeneous population of nucleic acids, in particular D-nucleic acids, whereby any of the nucleic acids comprises a region having a randomized sequence and a first constant sequence at the 5′ end and a second constant sequence at the 3′ end and whereby the nucleic acids forming the population differ in the randomized sequence, whereby the nucleic acid comprises modified nucleoside phosphates, preferably 2′-fluoro-modified nucleoside phosphates, and the constant sequences each comprises 4 to 6 nucleotides and the nucleic acid bears an OH group at the 3′ end,
(b) contacting the population of nucleic acids with the target molecule,
(c) separating the nucleic acids not interacting with the target molecule,
(d) separating from the target molecule the nucleic acid(s) interacting with the target molecule,
(e) optionally repeating steps (a) to (d), whereby the nucleic acid(s) of step (d) form the heterogeneous population or are contained therein,
(f) modifying the nucleic acid of step (a) or (d) by the following steps:
   (fa) phosphorylating the 5′ end of the nucleic acid under the proviso that the nucleic acid does not have a phosphate at the 5′ end,
   (fb) providing a first adapter molecule, whereby the first adapter molecule consists of a double-stranded nucleic acid of a first and a second nucleic acid strand, and whereby the first nucleic acid strand and the second nucleic acid strand are independent from each other a deoxyribonucleic acid, a ribonucleic acid or an FNA and whereby the 5′ end of the second nucleic acid strand provides for an overhang, whereby the overhang is at least partially complementary to the first constant partial sequence of the nucleic acid of step (a) and/or (d) or a part thereof,
   (fc) providing a second adapter molecule, whereby the second adapter molecule consists of a double-stranded nucleic acid of a first and a second nucleic acid strand, whereby the first nucleic acid strand carries a 5′ phosphate and the first and the second nucleic acid strand are independent from each other a deoxyribonucleic acid, a ribonucleic acid or an FNA, and whereby the 3′ end of the second nucleic acid strand provides for an overhang which is at least partially complementary to the second constant partial sequence of the nucleic acid of step (a) and/or (d) or a part thereof and whereby the second nucleic acid strand contains a cleavage site which, upon cleavage of the nucleic acid strand, provides for a first cleavage product and a second cleavage product, whereby the first cleavage product is the 3′ end of the second nucleic acid strand of the second adapter molecule which is at least partially complementary to the second constant partial sequence of the nucleic acid of step (a) and/or (d),
   (fd) ligating the first nucleic acid strand of the first end of the second adapter molecule to the nucleic acid of step (a) and/or (d), in order to obtain a ligation product as a reaction product,
(g) reverse transcription of the ligation product by using the second strand of the second adapter molecule present in the ligation reaction as a primer, in order to obtain a reverse transcription product,
(h) performing a second strand synthesis, whereby the second strand is essentially complementary to the reverse transcription product, whereby the second strand synthesis is preferably an amplification reaction and more preferably a polymerase chain reaction, and provides for an amplified reverse transcription product,
(i) degradation of the reverse transcription product, in particular of the amplified reverse transcription product, whereby a nucleic acid is provided which comprises in 3′→5′ direction:
   the sequence complementary to the forward primer or the forward primer binding site,
   the region complementary to the randomized region, as well as
   the region of the second strand of the second adapter molecule which is partially complementary to the second constant sequence at the 3′ end of the nucleic acid of step (a) and/or (d),
(j) performing a nucleic acid synthesis, whereby the nucleic acid provided in (i) is reacted with a forward synthesis primer, modified nucleoside triphosphates, preferably 2′-fluoro nucleoside triphosphates, and a reverse transcriptase, whereby the primer hybridizes to the complementary sequence of the first nucleic acid strand of the first adapter molecule, and the primer consists of RNA or of a combination of DNA and RNA, whereby the primer consisting of a combination of DNA and RNA has at least a ribonucleotide at its 3′ end, in order to obtain a synthesis product,
(k) cleaving off the reverse transcription product from the synthesis product of step (j) and of the forward synthesis primer sequence of the synthesis product of step (j), in order to obtain a nucleic acid which is essentially identical to the nucleic acid of step (a) or (d),
(l) optionally repeating steps (a) to (k), whereby the nucleic acid of step (a) forms the heterogeneous population or is contained therein, and
(m) optionally sequencing the nucleic acid obtained in step (h).

In an embodiment of the ninth aspect of the present invention the phosphorylating in step (fa) occurs by performing a kinase reaction.

In an embodiment of the ninth aspect of the present invention the cleavage site is provided by a restriction enzyme cleavage site and the cleavage occurs by a restriction enzyme.

In an embodiment of the ninth aspect of the present invention the cleavage site is provided by a ribonucleotide and the cleavage occurs via alkaline cleavage or via RNases.

In an embodiment of the ninth aspect of the present invention the cleavage in accordance with step (l) occurs in an enzymatic manner, preferably by DNase, and/or that the forward synthesis primer sequence is removed by an RNase.

In an embodiment of the ninth aspect of the present invention the nucleic acid of step (a) is a single-stranded nucleic acid consisting of modified nucleoside phosphates, in particular 2′-fluoro-modified nucleoside phosphates.

The problem underlying the present invention is solved in a tenth aspect by a method for the selection of a target molecule binding nucleic acid, in particular of aptamers, comprising the steps of
(a) providing a heterogeneous population of nucleic acids, in particular D-nucleic acids, whereby any of the nucleic acids comprises a region with a randomized sequence and a first constant sequence at the 5′ end and a second constant sequence at the 3′ end, and whereby the nucleic acids forming the population differ in the randomized sequence, whereby the first constant sequence comprises a forward primer sequence and the second constant sequence comprises a reverse primer binding site,
(b) contacting the population of nucleic acids with the target molecule,
(c) separating the nucleic acids not interacting with the target molecule,
(d) separating from the target molecule the nucleic acid(s) interacting with the target molecule,
(e) optionally repeating steps (a) to (d), whereby the nucleic acid(s) of step (d) form the heterogeneous population or are contained therein,
(f) second strand synthesis of a second strand complementary to the nucleic acid of step (a) and/or (d) and amplifying the second strand as well as the nucleic acids corresponding to the nucleic acid of step (a) and/or (d) by adding a reverse primer and a forward primer, whereby the reverse primer comprises a first and a second partial region, whereby the first partial region binds to the reverse primer binding site and the second partial region is arranged at the 5′ end of the reverse primer and comprises a promoter sequence for an RNA polymerase, whereby the synthesis product obtained by the second strand synthesis corresponds to the nucleic acid of step (a) and/or (d) and additionally has a sequence at its 3′ end which is complementary to the second partial region of the reverse primer,
(g) transcription of the synthesis product of step (f), whereby the transcription occurs upon addition of nucleoside phosphates and RNA polymerase and whereby the transcription product is subjected to a DNA digestion in order to obtain a transcription product which comprises in 3′→5′ direction the forward primer binding site, a region complementary to the randomized region of the nucleic acid of step (a), as well as a first partial region of the reverse primer,
(h) synthesis of a nucleic acid starting from the truncated transcription product of step (g), whereby the truncated transcription product is reacted with a forward synthesis primer, dNTPs and the reverse transcriptase, whereby the forward synthesis primer consists of deoxyribonucleotides,
(i) alkaline digestion of the reaction of step (h) for digesting the transcription product, in order to obtain a nucleic acid which is essentially identical to the nucleic acid of step (a) and (d),
(j) optionally repeating steps (a) to (i), whereby the nucleic acid(s) of step (i) forms the heterogeneous population or is contained therein, and
(k) optionally sequencing the nucleic acid(s) obtained in step (f) or (d).

In an embodiment of the tenth aspect of the present invention the nucleic acid of step (a) is a deoxyribonucleic acid.

In an embodiment of the tenth aspect of the present invention the promoter sequence is selected from the group comprising the promoter sequences of T7-RNA polymerase, T3-RNA polymerase and SP6 polymerase.

In an embodiment of the third, the fourth, the fifth, the sixth, the seventh, the eighth, the ninth and/or the tenth aspect of the present invention the selected nucleic acid(s) is/are selected from the group comprising aptamers, ribozymes, aptazymes, antisense molecules and siRNA.

The present inventors have surprisingly found that by means of a reverse transcriptase activity, which is also referred to herein in the following as reverse transcriptase, completely modified oligonucleotides and in particular completely 2′ F-modified oligonucleotides can be synthesized. For such purpose, only a reverse-complementary counterstrand of the sequence to be synthesized consisting of RNA or DNA or 2′-amino-modified nucleic acid or FNA, i.e. a 2′-fluoro-modified nucleic acid or a mixture thereof, and a suitable primer for the FNA synthesis are required which can hybridize to the counterstrand and which is referred to herein also as forward synthesis primer or FS primer. Preferably, this primer also consists of FNA. If such a primer was not available, then a primer consisting of RNA could be used which is, subsequent to the FNA synthesis, destroyed by RNases or alkaline hydrolysis. In such case the primer has to hybridize upstream, i.e. at the 5′ end of the sequence to be synthesized, at the counterstrand, which, optionally, has to have additional nucleotides at the 3′ end. As an alternative to the FS primer consisting of RNA, also a primer having the identical sequence but consisting of DNA may be used the 3′ terminal nucleotide of which is a ribonucleotide which is destroyed after FNA synthesis by RNases or alkaline hydrolysis. Optionally, the primer may contain further ribonucleotide moieties.

Using the method according to the present invention for the synthesis of a nucleic acid, in principle, nucleic acids can be prepared independent of their sequence and function. Functional nucleic acids which can be produced by using the method in accordance with the present invention for the synthesis of nucleic acids, comprise in particular also aptamers, ribozymes, antisense molecules and RNAi molecules. The molecules synthesis by using the method of the present invention have in common that they are, due to the modification, nuclease-resistant nucleic acids, which is particularly advantageous for the use in biological systems or processes. Biological systems as used herein are in particular embodiments among others: reactions with biological material, preparation of cells, tissue and organs, cells, tissues, organs and organisms, including, but not limited to, single-cellular or multi-cellular organisms, humans, animals and plants and samples thereof.

The method of the present invention also allows its implementation in the so-called SELEX process as, for example, described in European patent EP 0 533 838. It is thus possible by applying the method of the present invention for the synthesis of nucleic acids, to generate with the SELEX process target molecule binding oligonucleotides, nuclease-resistant aptamers and ribozymes, which completely consist of 2′-modified nucleotides. This extends their lifetime in an environment which contains RNases and DNases such as, for example, biological liquids and allow for an enlargement of their potential uses, for example prolonged lifetime in blood and other biological liquids, in the gastrointestinal tract, in cells and tissues.

Resistance to nucleases is also advantageous for antisense molecules which contribute to gene silencing (Manoharan, 1999). Antisense molecules are frequently chemically synthesized. There are, however, also methods which identify potentially successful antisense molecules by the use of enzymes (Xu et al., 2003; Zhang et al., 2003). By modifying such a method, also 2′ F-modified antisense molecules can be identified now. Additionally, 2′-F sequences have a higher melting point which results in stronger RNA-FNA or FNA-DNA duplices (Cummins et al., 1995).

This is also true for siRNAs. Also in connection therewith nuclease-resistance is advantageous so that the siRNAs reach their target, i.e. the RISC complex in the cytoplasm of their target cells, without being degraded (Chiu and Rana, 2003). By using the methods described herein completely 2′-modified siRNAs can be prepared in such a way in an enzymatic manner upon selection of appropriate templates and matrices.

The thus synthesized, completely 2′-modified nucleic acids are also suitable for, e.g. mass spectrometric analysis of nucleic acids. In connection with mass spectrometric analysis it has been found that there may be a loss of individual bases, whereby this loss can be reduced by the use of FNA. Because of this also longer sequences can be measured at high resolution if they are present as 2′-modified nucleic acids.

In summary, the methods of the present invention allow for a synthesis of nucleic acids having and consisting, respectively, of modified nucleoside phosphates, in particular 2′-F-modified nucleoside phosphates, whereby the portion of termination products is very small and whereby the portion of full length products is very high. A further advantage of the methods of the present invention is that the mutation rate is very low and that they allow for the incorporation of all and not only individual modified nucleotides.

Although the various embodiments are represented herein by reference to the use of 2′-fluoro-modified nucleoside phosphates, the methods of the present invention are not limited thereto, but are, in principle, applicable to all of the modified nucleoside phosphate described herein.

The reverse transcriptases used in the practice of the present invention are thus in preferred embodiments RNA-dependent reverse transcriptases which may, in connection with the present invention, also be used in combination with DNA matrices and FNA matrices.

As used herein in preferred embodiments, the term “reverse transcriptase” refers to any enzymatic activity which is, starting from a (+) RNA strand which is used as a template, suitable to synthesize a complementary (−) DNA strand. It is an RNA-directed DNA polymerase (Stryer, 1995). For the practising of the methods described herein, suitable reverse transcriptases are particularly also those summarized in the following table.

| | | | Temperature |
|---|---|---|---|
| Name | Provider | Organism | optimum |
| GeneAmpr rTth | ABI | Thermus thermophilus | 70° C. |
| Superscript II | Invitrogen | MMLV, RNase H⁻ | 42° C. |
| Superscript III | Invitrogen | MMLV, RNase H⁻ | 50° C. |
| ThermoScript | Invitrogen | AMV, RNAse H⁻ | 70° C. |
| C. therm | Roche | Carboxydothermus | 60-70° C. |
| | | hydrogenoformans | |
| MMLV | Epicentre | MMLV | |
| M-MuLV RT | Fermentas | MMLV | |
| AMV RT | CPG | AMV | |

It is within the present invention that also DNA polymerases can be used in connection with the methods of the present invention, provided that they exhibit the enzymatic activity required in the methods, i.e. the activity of a reverse transcriptase. Such DNA polymerases are, for example, bacterial DNA polymerases having reverse transcriptase activity such as the enzymes of Thermus thermophilus and Carboxyhydrothermus hydrogenoformans mentioned in the above table. Further particularly preferred reverse transcriptases are those which do not have any RNaseH activity.

About the use of the various reverse transcriptases disclosed herein it is to be noted that mixtures of the individual reverse transcriptases may be used. It is within the present invention that the individual reverse transcriptase is used in its wildtype form or in its mutated form or as a mixture thereof, either alone or together with one or several other reverse transcriptases whether in wildtype form or in mutated form or as a mixture thereof.

As used herein, the term “essentially” in connection with the description of a nucleic acid which essentially consists of a distinct species of nucleoside phosphates, indicates in an embodiment that the respective nucleic acid may consist completely of the respective nucleoside phosphates, but that, to a certain extent, also other nucleoside phosphates may be contained in the nucleic acid, namely those which do have a different or no modification. Examples that a distinct nucleic acid consists only essentially of distinct modified nucleoside phosphates can also be established by the starting materials, i.e. the individual used nucleoside phosphates not containing in each and any case the respective modification due to impurities. Furthermore, it is within the skills of the one of the art to determine to what extent those nucleoside phosphates may be contained in a nucleic acid which are different from the majority of the modified nucleoside phosphate contained in the nucleic acid.

In connection with the present invention all of the nucleoside triphosphates may be modified or only one respective species or, also, a distinct portion of an individual species may be modified or several species may be modified proportionally. Preferred nucleoside triphosphates are 2′-F-ATP, 2′-F-CTP, 2′-F-GTP, 2′-F-TTP and 2′-F-UTP as well as the nucleoside triphosphate of the universal base inosine 2′-F-ITP. These may be exchanged against 2′-dNTPs as desired.

Furthermore, in a preferred embodiment the term “essentially complementary to” refers to nucleic acids which are capable of hybridizing to each other, whereby it is preferred that they hybridize with each other under conditions of medium stringency and in particular under conditions of higher stringency. Conditions for medium stringency and high stringency, respectively, are, for example, described in Current Protocols or Maniatis et al. The stringency can be adjusted by selecting the incubation temperature and the concentration of cations accordingly.

As used herein the term “alkyl” refers to a methyl, ethyl and propyl group. Additionally, the term alkyl and “allyl”, respectively, also comprises an ethenyl and a propenyl group as well as methoxy, ethoxy and propoxy group.

As used herein, the term “nucleoside phosphate” refers in an embodiment of the present invention also to derivatives, in particular nucleoside thiophosphates, however, is not limited thereto.

It is within the methods for the selection of a target molecule binding nucleic acid disclosed herein that, among others, aptamers may be selected. Furthermore it is possible that by using these methods, also ribozymes, antisense molecules and RNAi molecules are selected, whereby the target molecule is different in accordance therewith. In case of ribozymes the characteristic of the ribozyme is to bind to the target molecule and to modify it or itself subsequently to the binding. The modification can be the forming or cleaving of one or several chemical bonds or both. In case of applying the selection methods of the present invention for the selection of antisense molecules the target molecule is a nucleic acid, in particular an mRNA or precursors thereof.

In a further aspect the present invention is also related to a method for the enzymatic synthesis of single-stranded DNA and in particular the use thereof in a method for the selection of single-stranded DNA or DNA oligonucleotides.

Single-stranded DNA oligonucleotides can—analogous to single-stranded RNAs—recognize target structures and bind thereto (Bock et al., 1992; Green et al., 1996; Leva et al., 2002). They are also referred to as DNA-aptamers. DNA-aptamers have, in contrast to those aptamers which contain ribonucleotides or consist thereof, the advantage that they are stable to alkali and can be autoclaved. This is a tremendous advantage in case the aptamer is, for example, used in chromatography columns as affinity matrix which are to be regenerated by “cleaning in place” procedures. In connection therewith diluted lyes are commonly used. For applications in the medical field such as, for example, extracorporal adsorber for blood dialysis sterility is an absolute requirement. Therefore, a sterilization is required, possibly also for re-use. The most common method is autoclaving. DNA aptamers survive this while staying intact, whereas RNA aptamers hydrolyse.

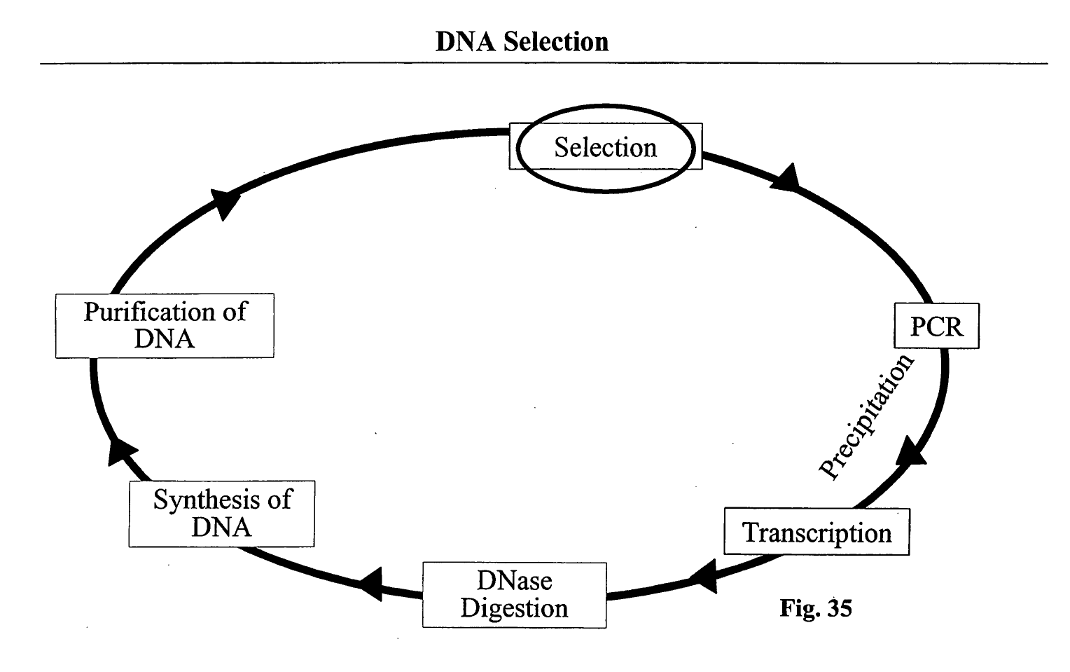

An advantage of the use of a DNA oligonucleotide in an amplification step in the context of the SELEX method as depicted in more detail in FIGS. 35 to 38, is, e.g., that compared to the prior art (Williams et al., 1997) PCR is not provided as the last amplification step. Therefore, a preparative gel purification of the DNA is not necessary, i.e. no gel purification and elution from the gel and precipitation as described in the prior art is necessary. Usually, both DNA strands which have been prepared in the PCR, are separated from each other, whereby one strand is truncated by the cleavage of a ribo primer prior to that. The preparative gel purification which is required according to the prior art and which cannot be automatised easily, is not necessary in the practice of the method of the invention for the amplification and selection of DNA oligonucleotides, in particular single-stranded DNA oligonucleotides, which is the reason for their particular suitability for use in automatised methods for performing selection methods such as the SELEX method.

As an alternative to the separation of the DNA strands obtained in the PCR by gel purification, the use of a biotinylated primer is described in the prior art. In such case the PCR product is immobilized on streptavidine beads and the strand which does not contain the biotinylated primer, will be eluted by means of sodium hydroxide solution, heating or similar measures. The capacity of the bead is frequently very limited as also biotinylated, but non-incorporated primers occupy the biotin binding sites. Also, the beads frequently suffer from the alkaline conditions and heat, respectively, so that streptavidine or other bead constituents are eluted together with the DNA strand. These have to be removed subsequently again by, e.g., phenol chloroform extraction which cannot be automatised easily.

A further advantage of the method of the invention is that the DNA for each following selection round is not only prepared by preparative PCR, but also by PCR and in vitro transcription. Therefore, less PCR cycles are necessary. As the PCR exerts a high selection pressure and as in connection with high cycle numbers PCR artifacts, so-called amplification parasites, occur frequently (Murphy et al., 2003), this is of great value.

The present invention shall be illustrated in the following referring to the following figures and examples, from which further features, embodiments and advantages may be taken. It is particularly mentioned that individual features as disclosed in the context of further features as described in connection with the subsequently described figures and examples, may be used as such individually also in other embodiments of the present invention.

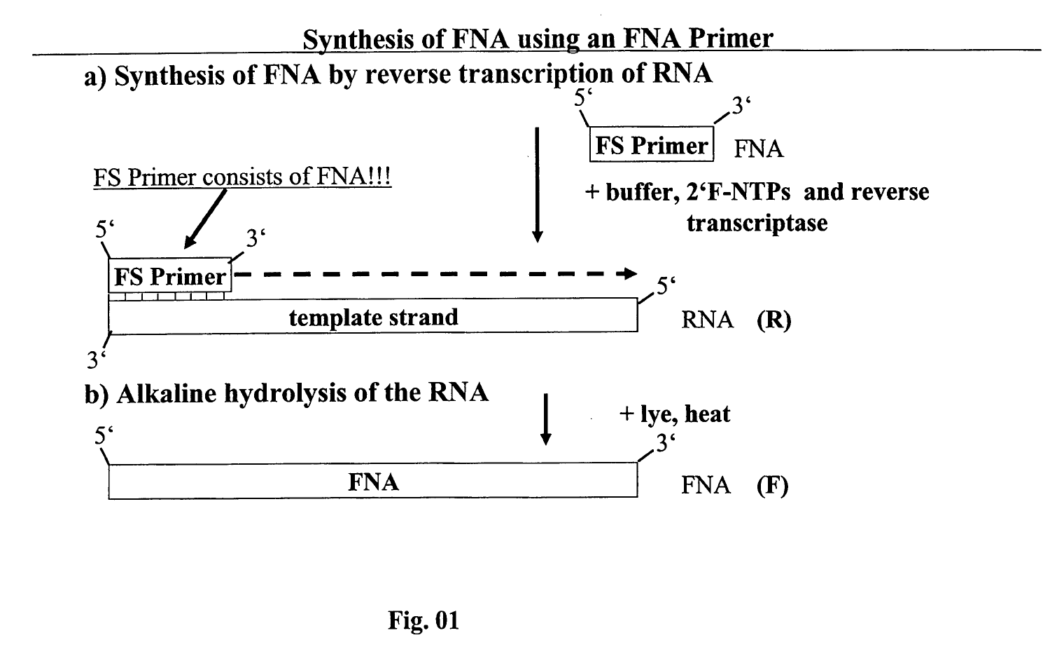

FIGS. 1 to 3 show the schematic course of different embodiments of the method of the invention for the synthesis of nucleic acids;

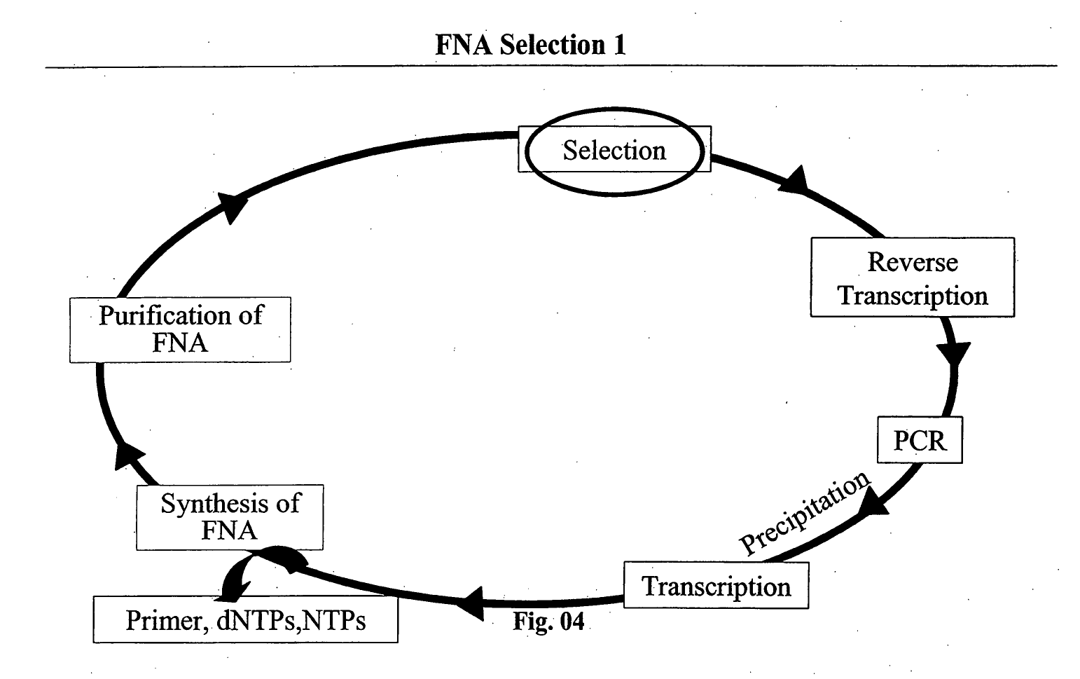

FIGS. 4 to 38 show schematic courses of different embodiments of the method of the present invention for the selection of a target molecule binding nucleic acid using the method of the invention for the synthesis of nucleic acids;

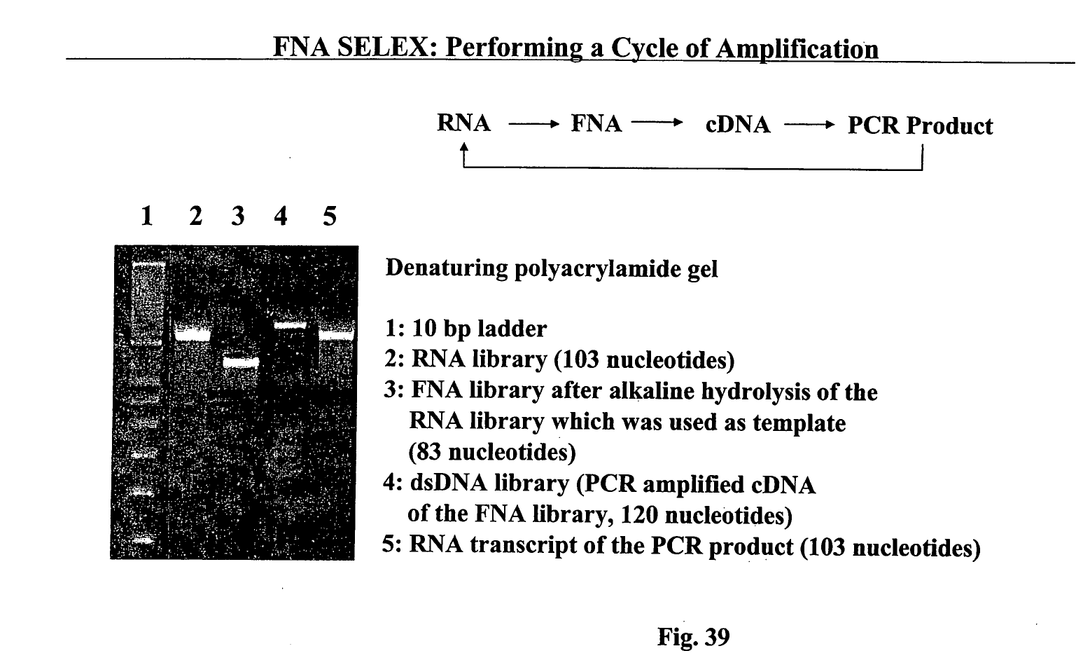

FIG. 39 shows schematically the performance of an amplification cycle using the method of the invention for the synthesis of nucleic acids and the result of an analysis of the method by means of PAGE;

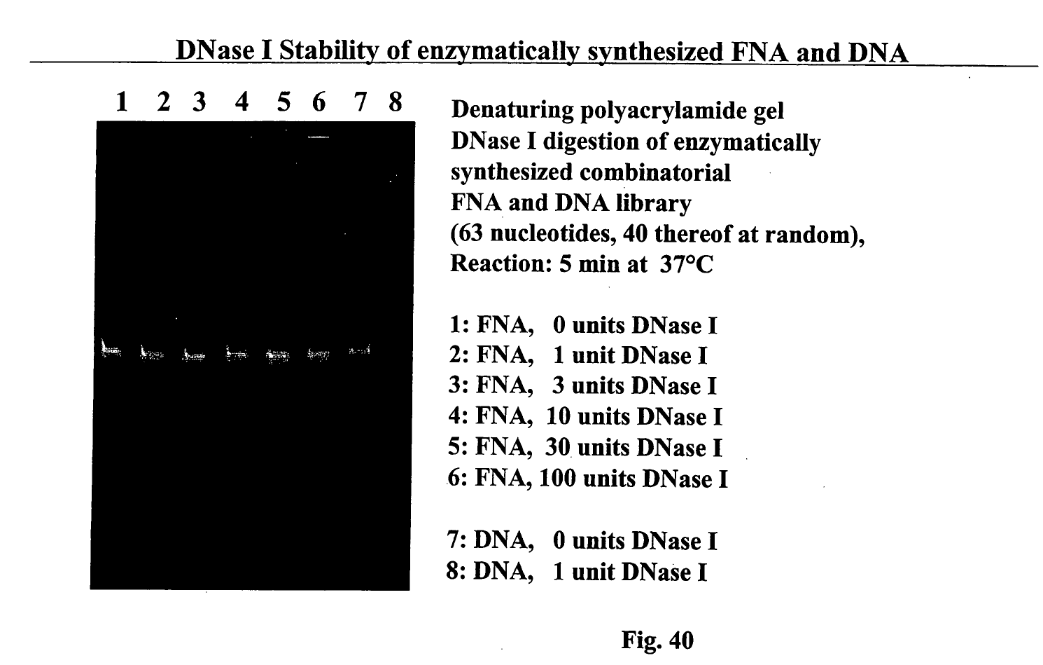

FIG. 40 shows the result of a PAGE analysis on the stability of enzymatically prepared combinatorial FNA and DNA libraries;

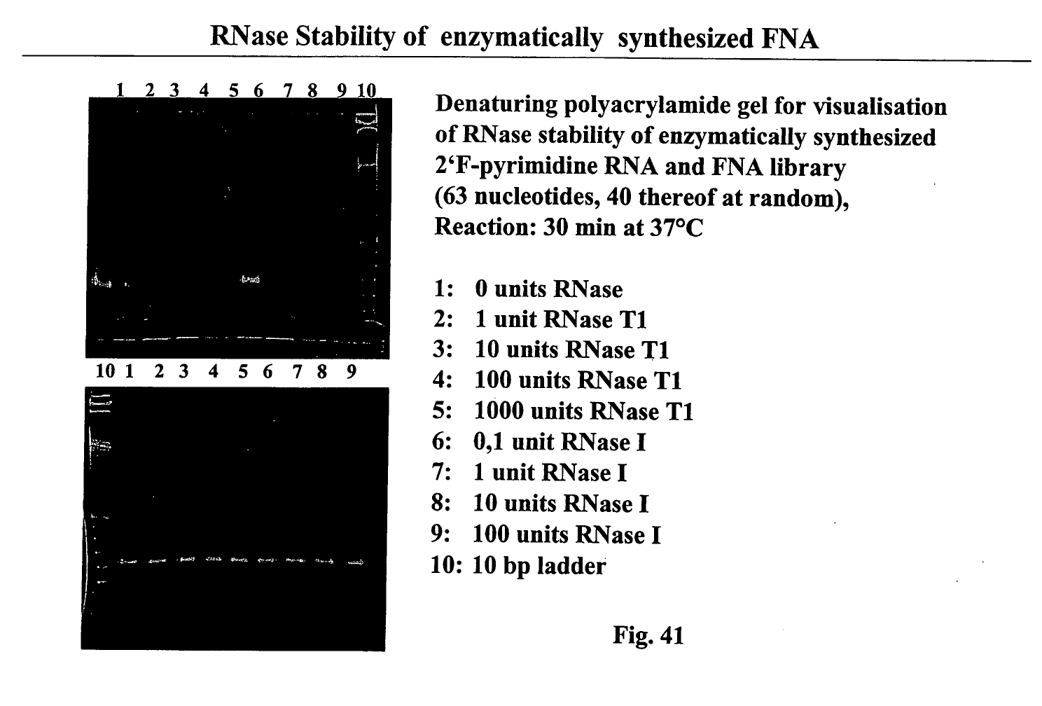

FIG. 41 shows the result of a PAGE analysis on the stability of enzymatically prepared combinatorial FNA and 2′-F-pyrimidine RNA libraries compared to RNase T1 and RNase I;

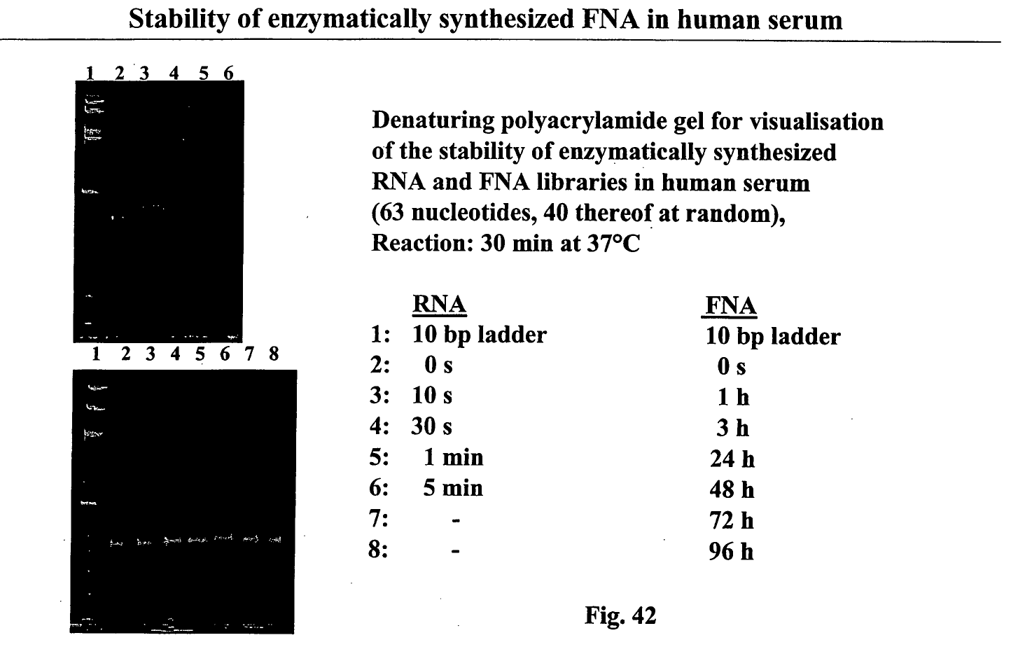

FIG. 42 shows the result of a PAGE analysis on the stability of enzymatically prepared combinatorial FNA and RNA libraries in human serum; and

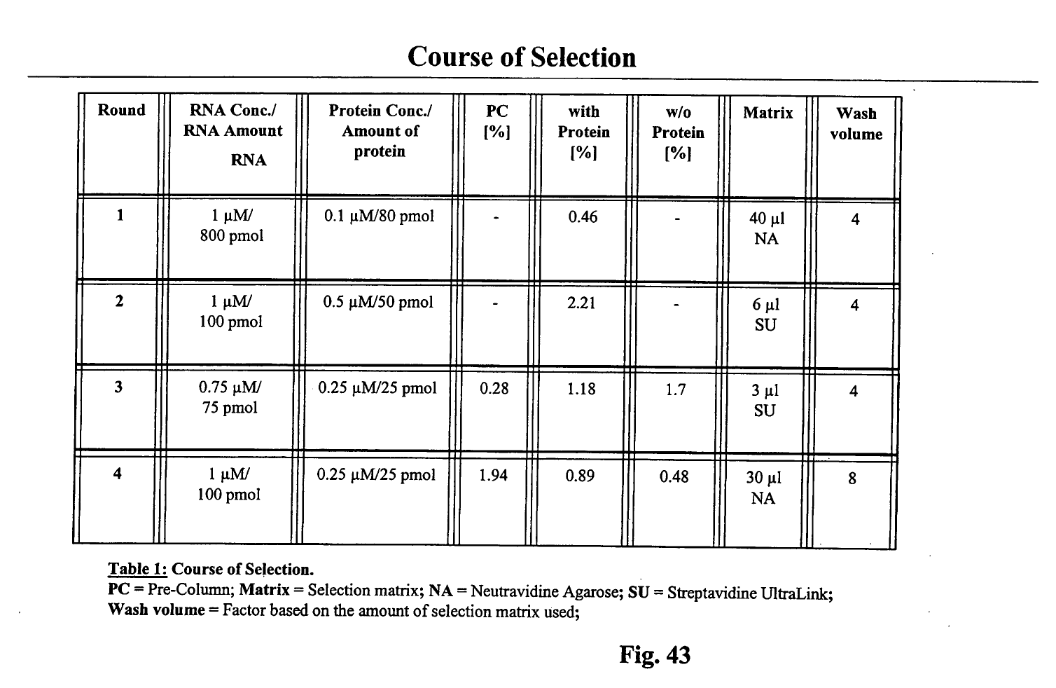

FIG. 43 shows the selection course of the in vitro selection described in example 1.

FIG. 1 shows the basic course of a synthesis of nucleic acids, in particular FNA, using a reverse transcriptase. More specifically, starting from a template strand of RNA, a primer is annealed or hybridized to the 3′ end of the template strand consisting of RNA, such that a free 3′ OH end is available which is extended under the influence of the reverse transcriptase and the 2′-F-NTPs used in the particular example, and the addition of an appropriate buffer to the 5′ end of the template strand, accordingly. The primer is in the present case a FNA. The double-stranded nucleic acid molecule obtained in this way is subsequently further treated to remove the template strand. Such treatment can be a cleavage under alkaline conditions and heat (300 mM sodium hydroxide solution, 10 min at 95° C.) which results in digestion of the RNA constituents of the double-stranded nucleic acid and only leaves the synthetic nucleic acid strand which, in the present case, consists completely of FNTP.

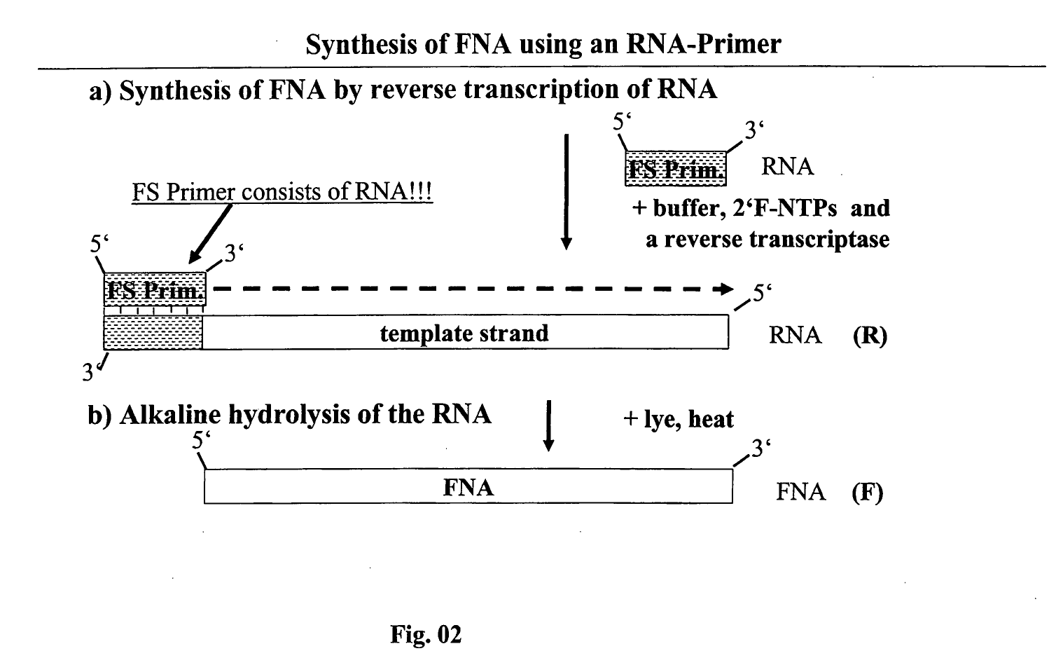

FIG. 2 shows an alternative method of FIG. 1 for the synthesis of a nucleic acid, in particular of FNA. In this embodiment a primer consisting of RNA is used. The template sequence consisting of RNA must in this case be extended at its 3′ end by further nucleotides so as to allow for the RNA primer to hybridize thereto. Preferably this extension comprises a stretch of 10 to 25 nucleotides. After reverse transcription by means of a reverse transcriptase, the template strand and the RNA primer present at the synthesized nucleic acid strand are removed, for example by alkaline hydrolysis or an RNase digestion.

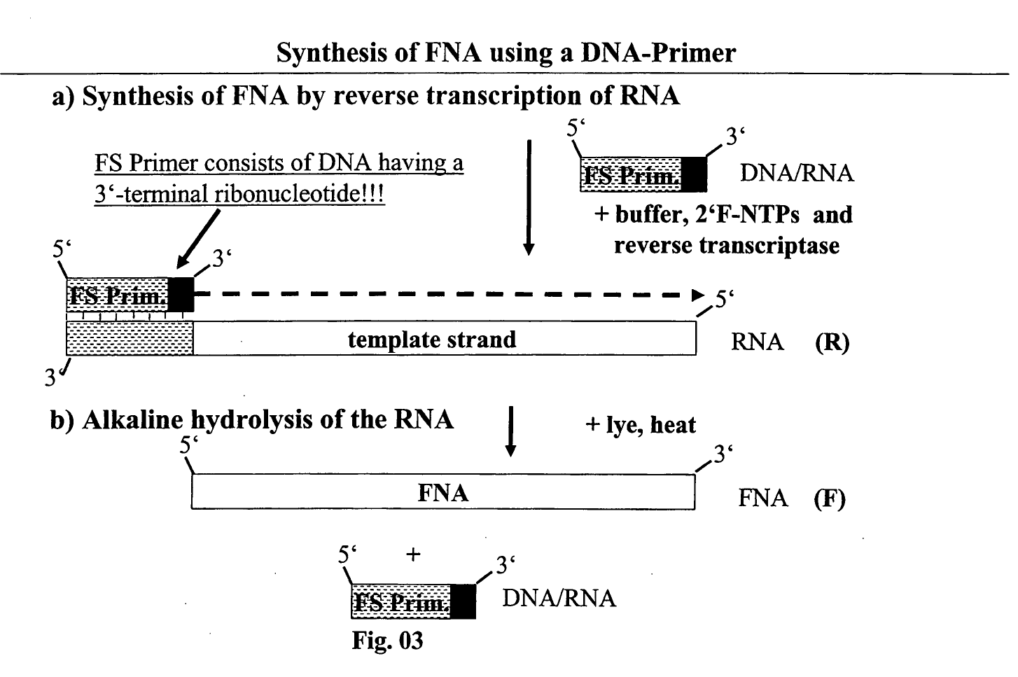

FIG. 3 shows a further embodiment of the method of the invention for the synthesis of a nucleic acid as depicted in FIG. 1. Similar to the method described in FIG. 2 a primer is used which is different from an FNA primer. Specifically, a DNA primer having at least one ribonucleotide is used. The DNA primer can additionally contain further ribonucleotides, whereby, however, it is essential that a ribonucleotide is present at the 3′ end of the DNA primer. Also in this case the template strand consisting of RNA comprises a further sequence at its 3′ end which allows for the hybridization of the primer thereto. The RNA template strand is removed by the methods and measures described in connection with FIGS. 1 and 2. The primer is removed from the newly synthesized FNA strand by means of alkaline hydrolysis of the ribonucleotide attached to the 3′ end, and can be subsequently removed, for example, by gel purification, molecular sieve or gel filtration.

FIG. 4 shows a schematic for the performing of an in vitro selection process which integrates the method of the invention for the synthesis of a nucleic acid. After the selection of suitable FNA species these are transcribed into complementary DNA (cDNA) by reverse transcription using dNTPs. For the amplification of the sequences a PCR and an in vitro transcription are performed next. Prior to the subsequent FNA synthesis the dNTPs, NTPs and primers are removed which originate from the PCR and in vitro transcription and which interfere with FNA synthesis. After the FNA synthesis the FNA is purified by, for example, denaturing PAGE or alkaline hydrolysis of the template strand. During the alkaline hydrolysis of the template strand, the forward synthesis primer can be cleaved off from the FNA at the same time, provided that it does not also consist of FNA.

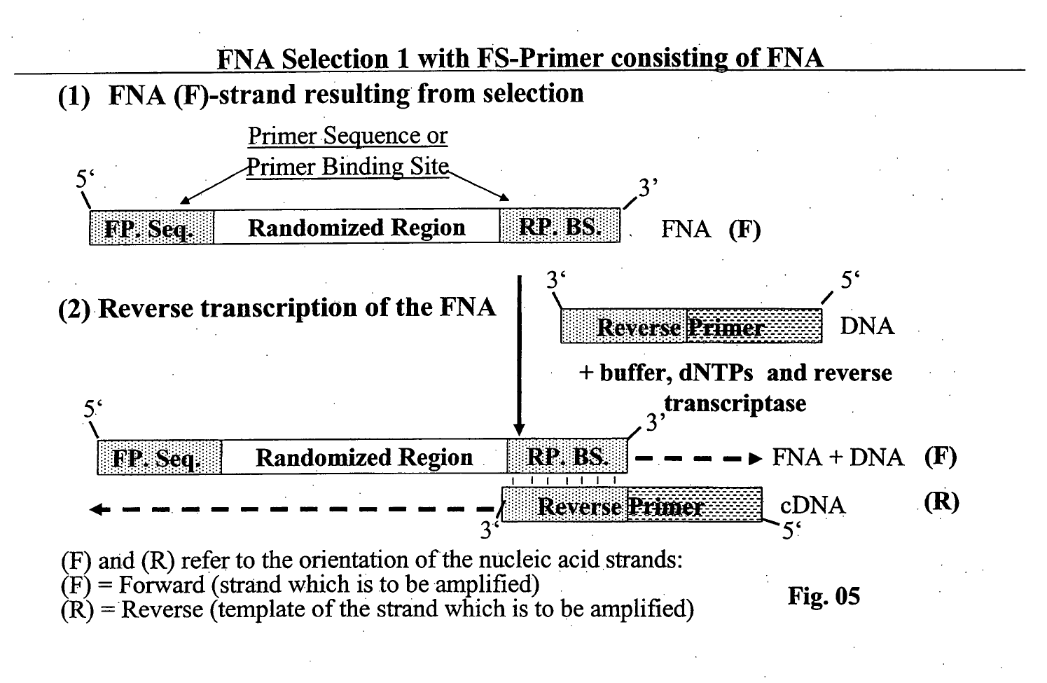

FIGS. 5 to 8 show the course of an amplification method with a transcription step as, for example, can be used in an in vitro selection process as depicted in FIG. 4.

In particular FIG. 5 shows the synthesis of cDNA by means of reverse transcriptase such as, for example, Superscript II (Invitrogen) starting from the selected 2′-F oligonucleotides. These 2′-F oligonucleotides may represent the heterogeneous population of nucleic acids as used in an in vitro selection process. The oligonucleotides which are also referred to herein as starting nucleic acid, have a first constant sequence at the 5′ end and a second constant sequence at the 3′ end flanking the randomized region or the randomized sequence. The first constant sequence of the nucleic acid comprises a forward primer sequence and the second constant sequence comprises a reverse primer binding site. It is preferred that the first constant sequence is the forward primer sequence and the second constant sequence is the reverse primer sequence. The reverse primer composed of DNA consists of two partial regions. The first partial region contains or comprises the sequence binding to the reverse primer binding site, and the second partial region contains or comprises a promoter for an RNA polymerase. In a reaction comprising the reverse primer, buffer as well as dNTPs and a reverse transcriptase, a cDNA strand is thus formed and the starting strand, i.e. the fluoro strand is extended by dNTPs.

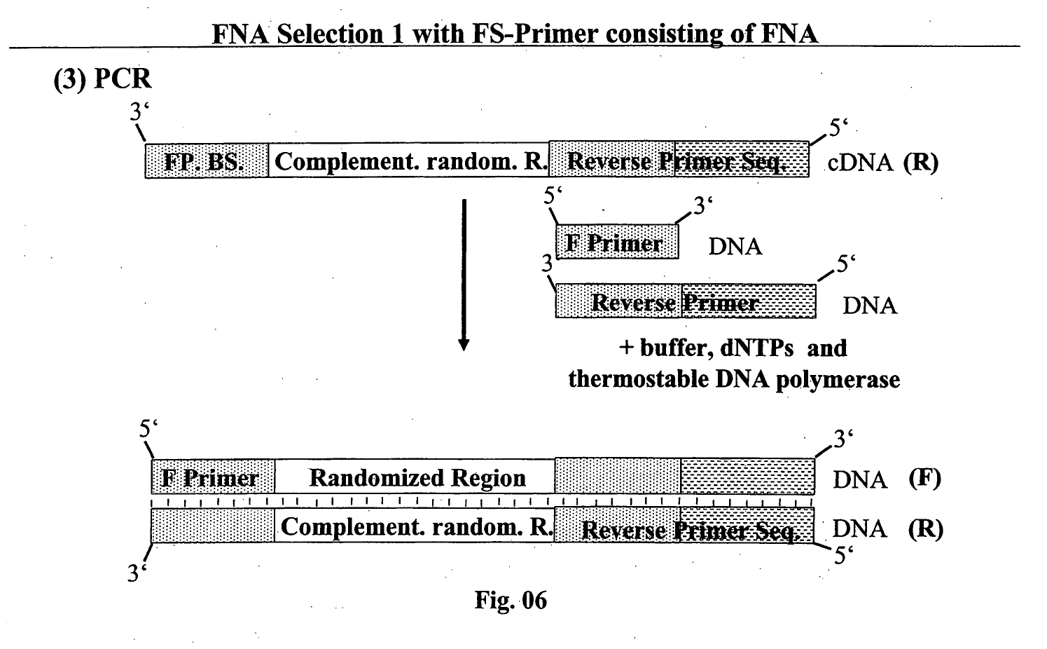

FIG. 6 shows the course of a second strand synthesis step which is embodied as a polymerase chain reaction. In connection therewith there is an amplification of the cDNA and in particular also an amplification of the reverse transcription product which is referred to in FIG. 5 also as “R” strand. Apart from the reverse primer additionally a forward primer and a DNA polymerase, preferably a thermostable DNA polymerase, are used. The F-primer binds to the F-primer binding site of the reverse transcription product and allows the synthesis of the second strand starting therefrom.

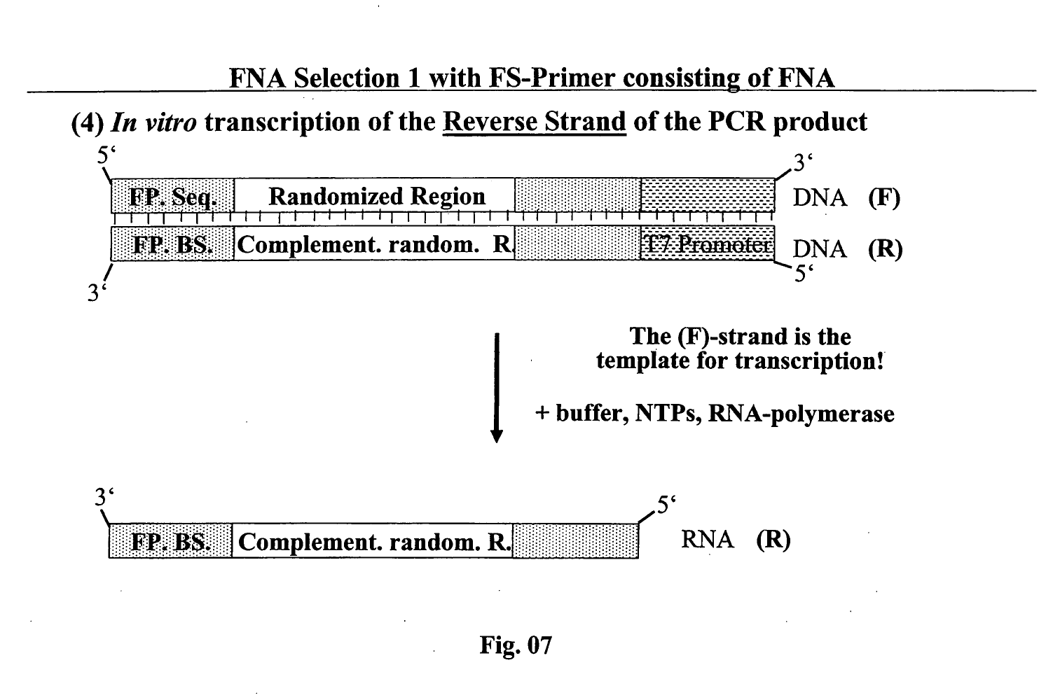

FIG. 7 shows an in vitro transcription of the reverse strand of the PCR product as RNA the synthesis of which is described in FIG. 6. As a template for this transcription the forward strand of the PCR product referred to as “F” is used. The RNA polymerase promoter region, in the particular region a T7 promoter, is not transcribed. As a result, a ribonucleic acid is obtained which is complementary to the used nucleic acid as depicted in partial step (l) of FIG. 5, i.e. the starting nucleic acid.

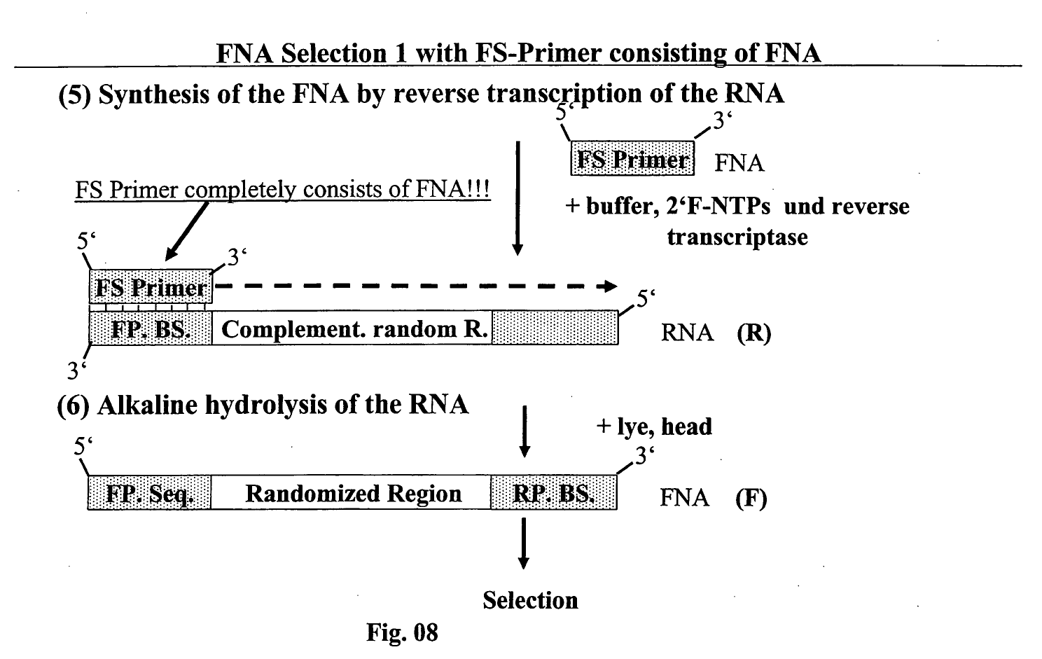

FIG. 8 finally shows the FNA synthesis using an FNA primer which is referred to herein as FS primer. The reaction contains, apart from the FS primer, 2′-F-NTPs as well as a reverse transcriptase such as, for example, Superscript II of Invitrogen. The forward synthesis primer hybridizes to the forward primer binding site of the transcription product consisting of RNA and which also serves as template strand (FIG. 7) and which allows the synthesis of an FNA nucleic acid strand which is essentially complementary to the sequence of the reverse transcription product which has been amplified in the meantime. The template strand is, in the present case, removed by alkaline hydrolysis or an RNase digestion. Optionally, FNA purification as well as labeling and optionally a further selection round follow.

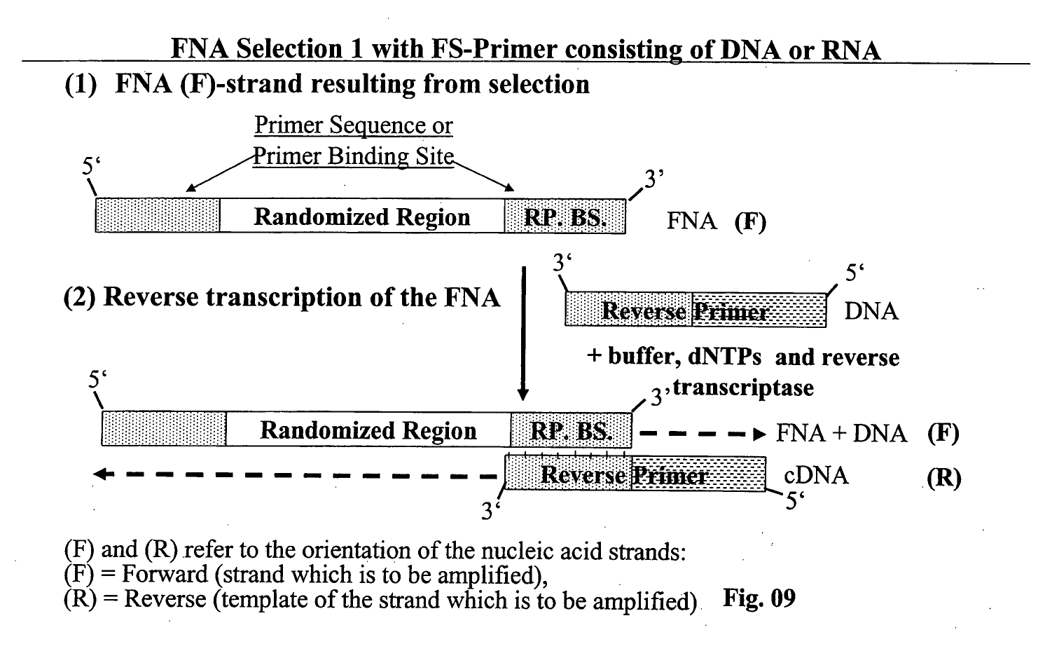

FIGS. 9 to 12 show a method for a possible amplification with a transcription step, whereby an RNA or RNA-DNA primer is used instead of a primer consisting of FNA. The reaction course is essentially identical to the reaction course depicted in FIGS. 5 to 8, whereby, however, there are some differences as presented in the following.

FIG. 9 shows a reverse transcription of the selected FNA molecule, i.e. the starting nucleic acid, which is performed under the same conditions as the reaction shown in FIG. 5.

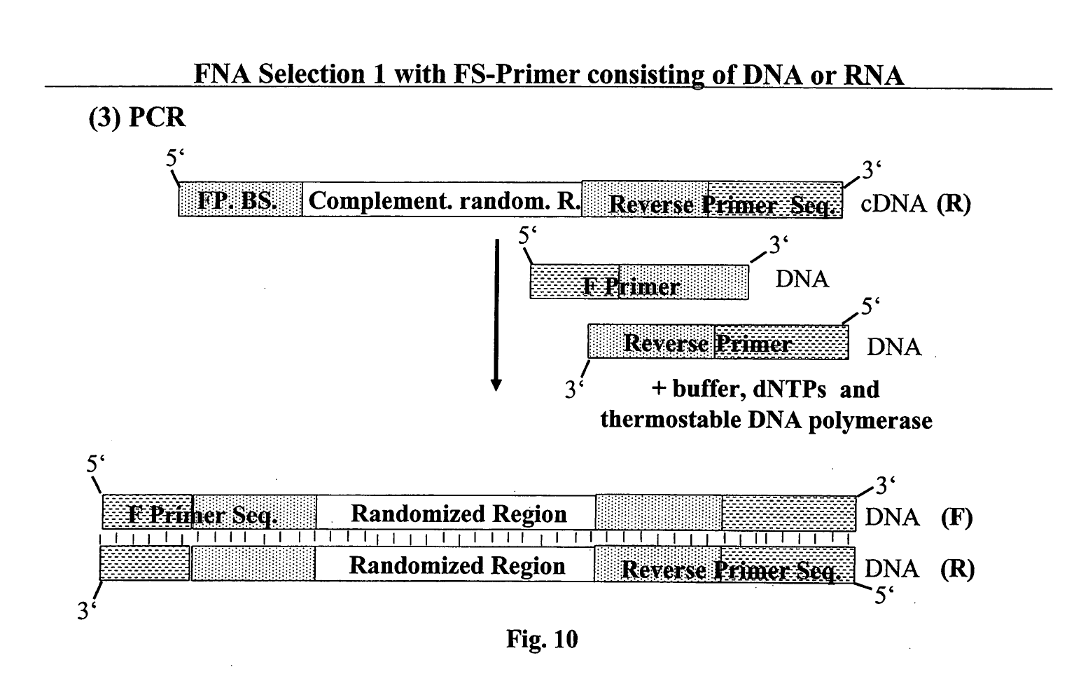

FIG. 10 shows the PCR amplification of the cDNA with the reverse primer and a forward primer which, first, comprises a sequence at the 3′ end which is essentially complementary to the forward primer binding site of the reverse transcript, and additionally comprises a further partial region at the 5′ end, whereby this further partial region corresponds to the sequence of the forward synthesis primer as used for the reverse transcription in connection with the FNA synthesis.

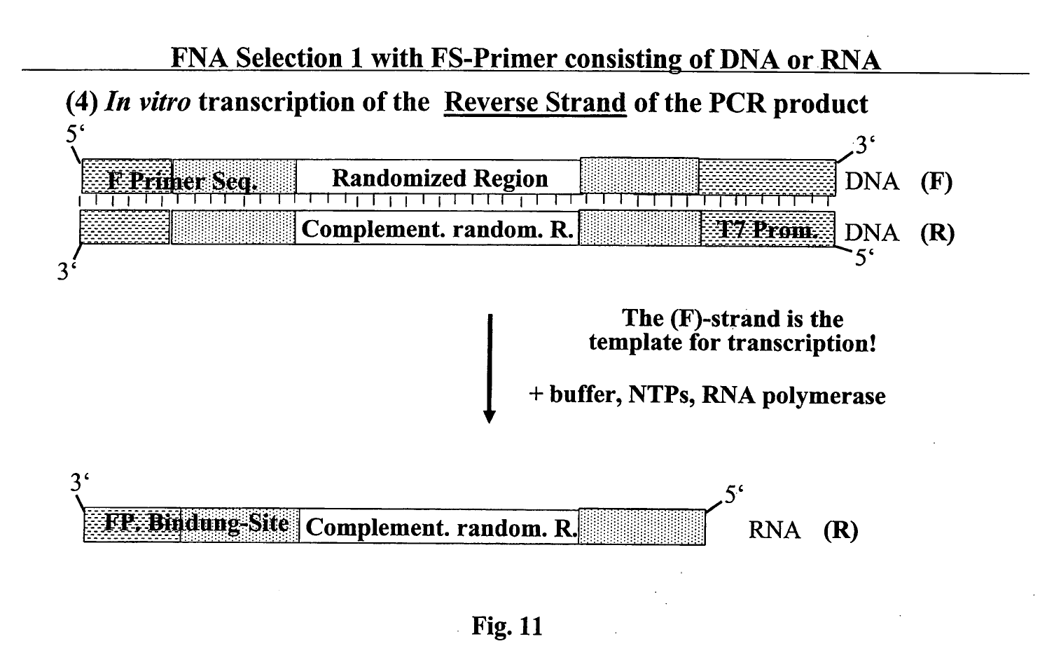

FIG. 11 shows the in vitro transcription of the PCR product counterstrand. The (F) strand is used as a template. A suitable RNA polymerase such as, for example, T7-RNA polymerase, and NTPs are added to the reaction and a transcription product is obtained which comprises in 3′→5′ direction a forward primer binding site, the region complementary to the randomized region as well as a reverse primer sequence. As a result this strand is extended on the one hand at its 3′ end compared to the complementary strand of the nucleic acid used in FIG. 9 (step (l), (F)) by the region which is complementary to the further region of the forward primer and, on the other hand, the region of the reverse primer which, at the 5′ end, extends beyond the region complementary to the reverse primer binding site, is not contained in the molecule.

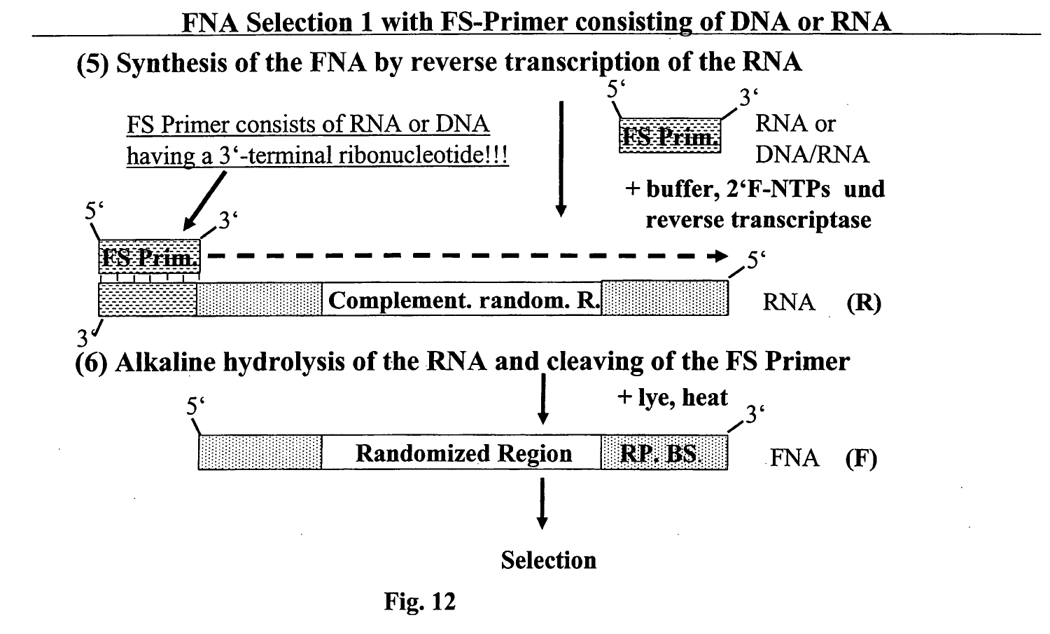

FIG. 12 shows the FNA synthesis with a reverse transcriptase such as, for example, Superscript II (Invitrogen) and 2′-F-NTPs. A ribonucleotide or a deoxyribonucleotide having at least one 3′ terminal ribonucleotide is used as a primer the sequence of which is identical to the sequence of the 5′ overhanging region of the forward primer, i.e. the further partial region of the forward primer. The template strand and the primer are separated from the FNA by alkaline hydrolysis. This single-stranded FNA nucleic acid can then be used in further selection rounds.

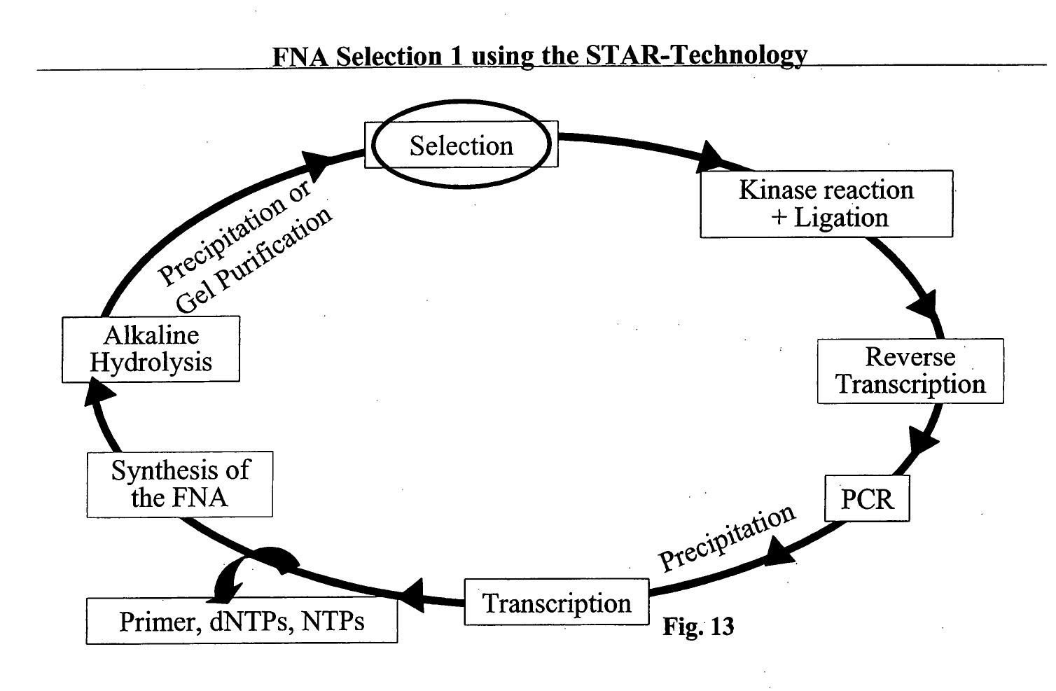

FIGS. 13 to 18 show the procedure for amplification of FNAs in a selection scheme, where the starting nucleic acid, apart from the randomized region, comprises only comparatively short regions flanking the randomized region, which is advantageous in the selection and allows in particular the truncation of the target molecule binding nucleic acids primarily obtained in a selection. This special embodiment of the selection method is also referred to herein as primer ligation (STAR-FNA selection 1).

FIG. 13 shows an overview of a cyclic process consisting of selection, primer ligation, amplification, FNA synthesis and FNA purification.

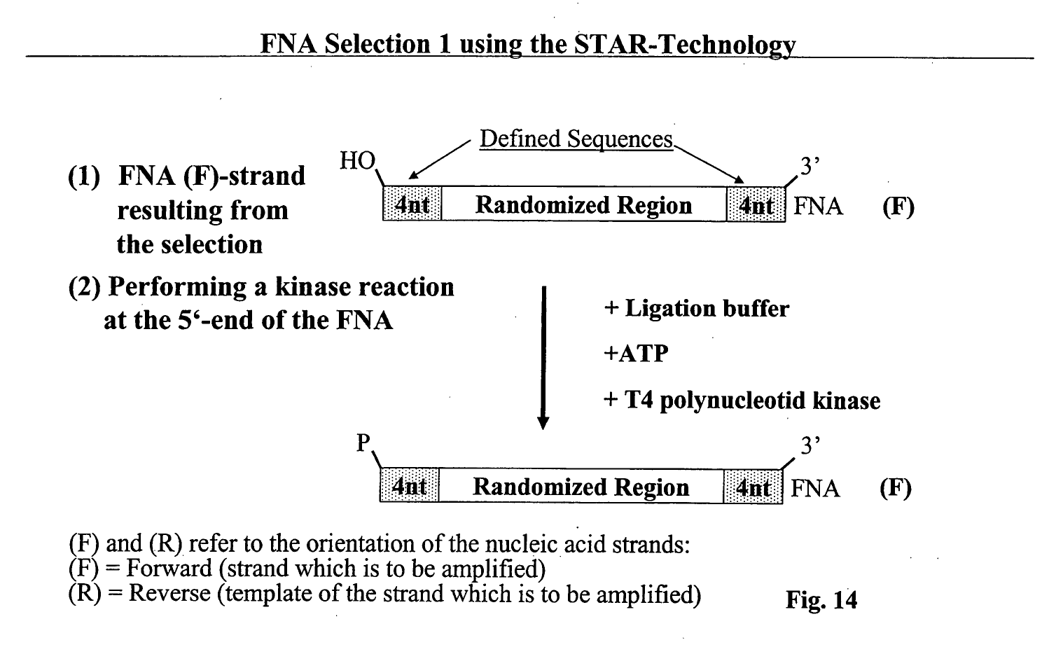

FIG. 14 shows in (l) the nucleic acid typically used as starting material in a selection process. The nucleic acid, in the present case, consists of FNA and has, apart from the randomized region at the 5′ end, a partial region which comprises a first constant sequence and at the 3′ end a further partial region containing a second constant sequence. The lengths of these sequences are in principle not limited in the methods described herein. However, a length of 4, 5 or 6 nucleotides is preferred, whereby more preferably the lengths of both constant sequences are identical; in principle, however, they may also be different. The nucleic acids forming the heterogeneous population differ in the randomized sequence. The nucleic acid may comprise at its 5′ end an OH group or a phosphate. The phosphate is of critical importance for the subsequent steps and is typically introduced at the 5′ end of the nucleic acid after the selection step by phosphorylation. The 5′ phosphorylation occurs in a suitable ligation buffer upon addition of ATP and, for example, a T4 polynucleotide kinase, whereby also other enzymes are suitable for such kinase reaction. In order to obtain in the subsequent steps a yield as high as possible, a kinase reaction as complete as possible is desired.

The nucleic acid having at its 5′ end a phosphate group, is subsequently modified. This modification uses two adapter molecules. The first adapter molecule consists of a double-stranded nucleic acid of a first and a second nucleic acid strand, whereby the first strands of the first and of the second adapter molecules are DNA, RNA or FNA, preferably DNA, the second nucleic acid strand is a deoxyribonucleic acid and the 5′ end of the second nucleic acid strand provides for an overhang, whereby the overhang is at least partially complementary to the first constant partial sequence of the nucleic acid of step (a) and/or (d), i.e. the starting nucleic acid. The second adapter molecule also consists of a double-stranded nucleic acid and also comprises a first and a second nucleic acid strand, whereby the first nucleic acid strand carries a 5′ phosphate and the second nucleic acid strand is a deoxyribonucleic acid and the 3′ end of the second nucleic acid strand provides for an overhang which is at least partially complementary to the second constant partial sequence of the starting nucleic acid shown in FIG. 14 (1) and FIG. 14 (2), respectively. Preferably both the first as well as the second nucleic acid strand of both the first and the second adapter molecule are made of deoxyribonucleic acid.

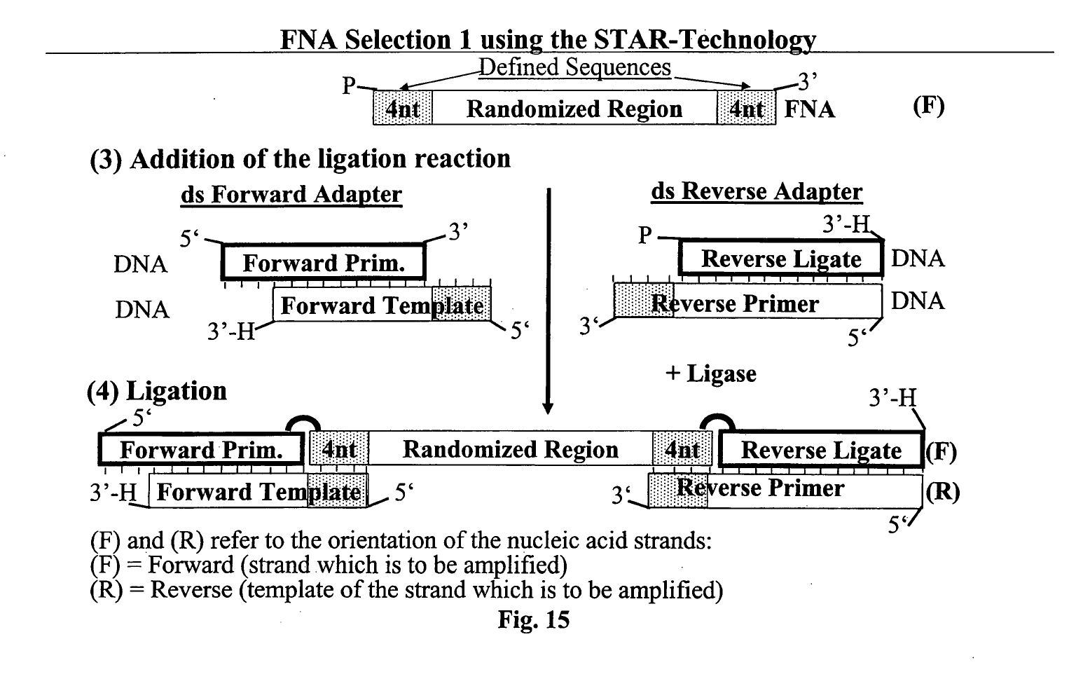

FIG. 15 shows the ligation of the first and the second adapter molecule to the nucleic acid which is used in the selection process as starting nucleic acid. The first nucleic acid strand of the first and the second adapter molecule is ligated to the starting nucleic acid by using a ligase. A suitable ligase is, for example, the T4-DNA ligase (MBI Fermentas). The thus obtained ligation product consists of the starting nucleic acid to the 5′ end of which the first nucleic acid strand of the first adapter molecule, also referred to herein as forward primer, is covalently bound and to the 3′ end of which the first nucleic acid strand of the second adapter molecule, also referred to herein as reverse ligate, is bound. The respective second nucleic acid strands of the first and of the second adapter molecule are, in accordance with their complementarity, hybridized to the corresponding first nucleic acid strand of the first and the second adapter molecule and with their corresponding overhangs to the corresponding first and second partial sequence of the starting nucleic acid. The reverse primer contains a promoter for an RNA polymerase which is located 5′ to the overhanging sequence comprising in the present case four nucleotides.

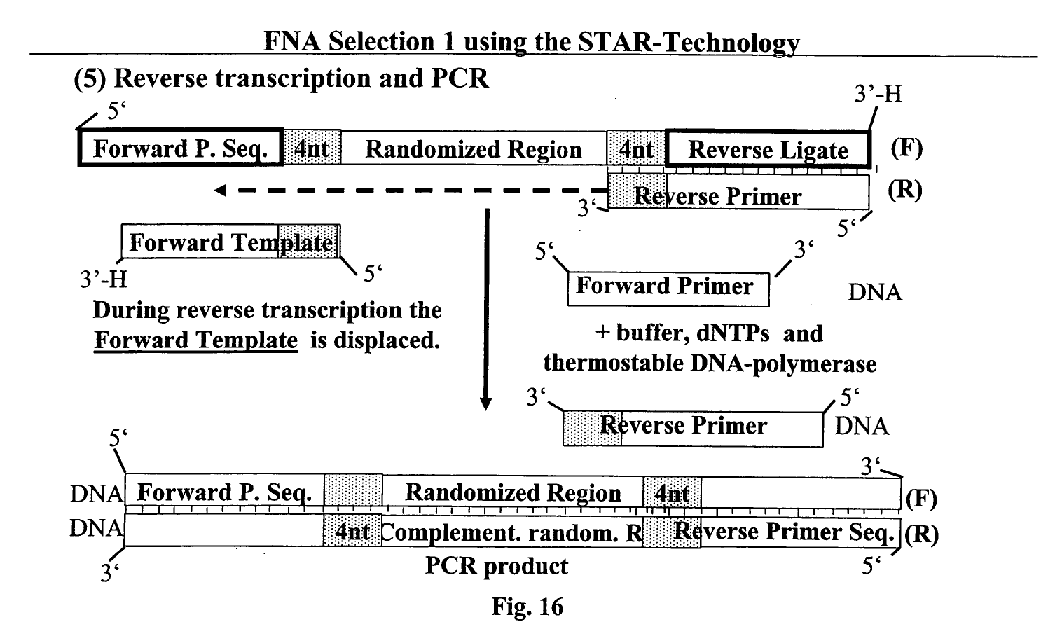

FIG. 16 shows the reverse transcription of the (F)-strand, i.e. the starting nucleic acid extended by the respective first nucleic acid strand of the first and the second adapter. All reverse transcriptases as described herein may be used as reverse transcriptases for such purpose, for example, Superscript II (Invitrogen). The synthesis of the starting nucleic acid which is extended by the two respective first nucleic acid strands, starts from the 3′ end of the reverse primer. The second nucleic acid strand of the first adapter molecule dissociates during reverse transcription. Subsequently, and upon addition of a suitable buffer as well as dNTPs, an amplification reaction such as a polymerase chain reaction is performed using suitable DNA polymerases. Thermostable DNA polymerases such as, for example, the Taq-DNA polymerase (Roche), are particularly preferred for such purpose. This amplification reaction results in an amplification of both the extended starting nucleic acid as well as, due to the reverse transcription, the nucleic acid strand complementary thereto. Both strands are preferably hybridized.

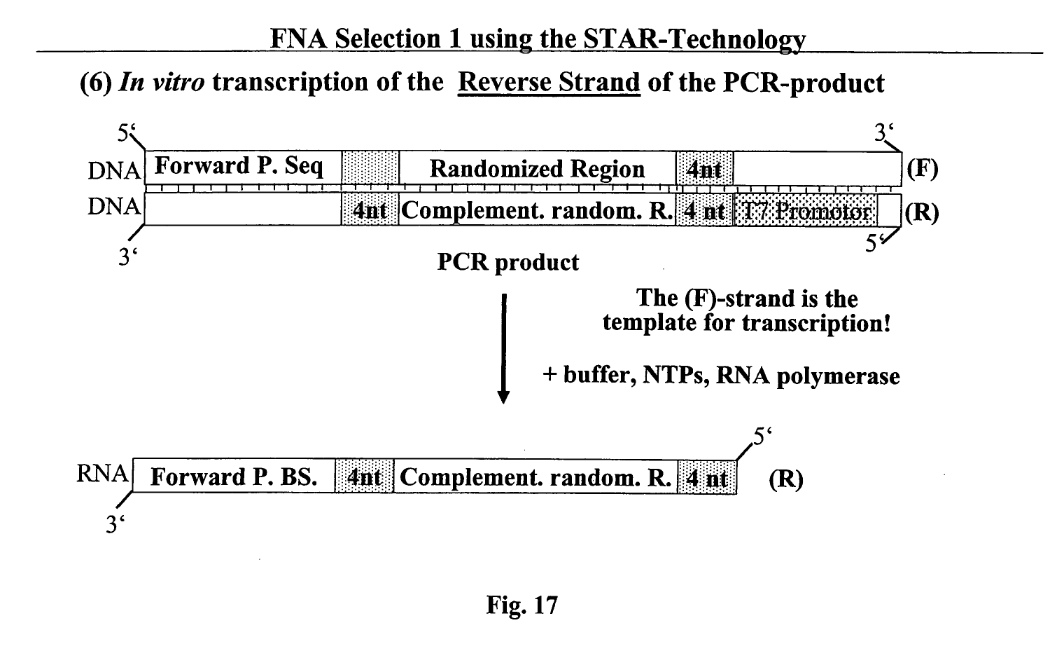

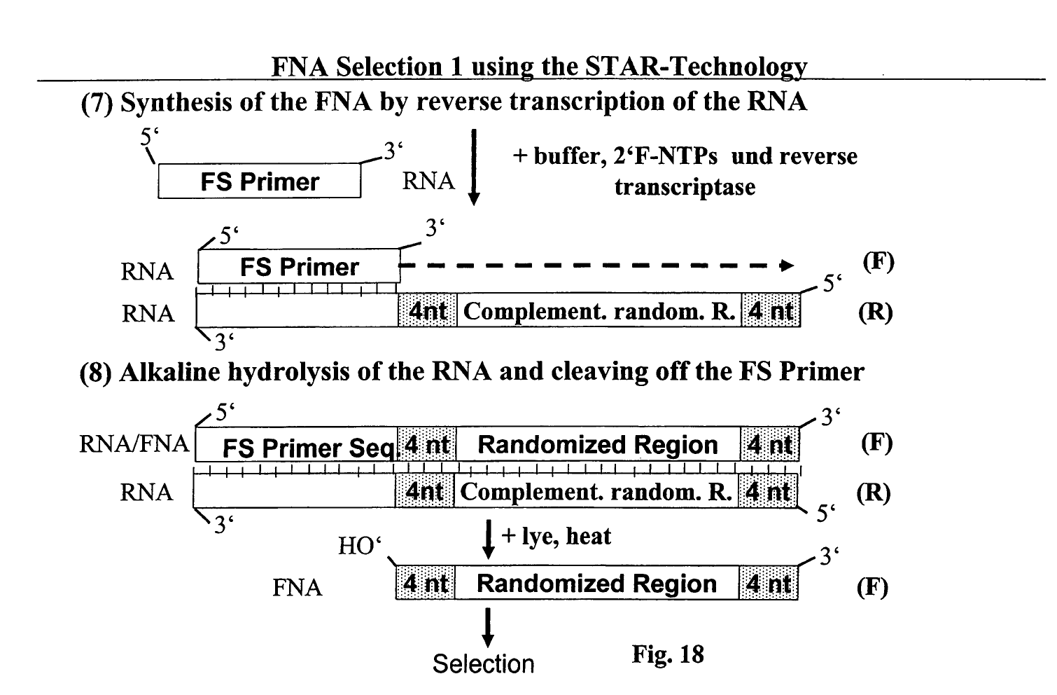

The reverse strand of the double-stranded amplification product is, as depicted in FIG. 17, subject to an in vitro transcription. The extended starting nucleic acid serves as a template for the transcription which is performed upon addition of suitable buffers, NTPs as well as an RNA polymerase. At the end, a transcription product is obtained which comprises in 3′→5′ direction a sequence which is complementary to the first strand of the first adapter molecule, a sequence which is complementary to the first constant partial sequence of the starting nucleic acid, a sequence which is complementary to the randomized region of the starting nucleic acid, and a region which is complementary to the second constant partial sequence of the starting nucleic acid. This product is subsequently subjected to a nucleic acid synthesis (FIG. 18) adding a forward synthesis primer consisting of RNA, which is also referred to herein as FS primer having the sequence of the first nucleic acid strand of the first adapter molecule, modified nucleoside phosphates, in the present case 2′-F-NTPs, as well as a reverse transcriptase, whereby all reverse transcriptases disclosed herein are, in principle, suitable reverse transcriptases. At the end of the reaction a double-stranded nucleic acid is obtained, whereby one strand corresponds to the transcription product and the other is complementary thereto. The transcription product consists of ribonucleic acid, whereas the synthesis product synthesized by the reverse transcriptase consists in the region of the FS primer of RNA and the subsequent region of modified nucleoside phosphates, in particular 2′-F nucleoside phosphates. Alternatively, a primer having an identical sequence consisting of DNA and the 3′ terminal nucleotide of which is a ribonucleotide, can be used instead of the FS primer consisting of RNA. The RNA components of this double-stranded nucleic acid are digested, for example, by alkaline cleavage or RNase activity, and ultimately a single-stranded nucleic acid is obtained which corresponds to the starting nucleic acid or, due to possible incorporation errors of the used enzymes, corresponds essentially thereto. The thus obtained nucleic acid can again be introduced into the selection process and can be used as starting nucleic acid there, respectively.

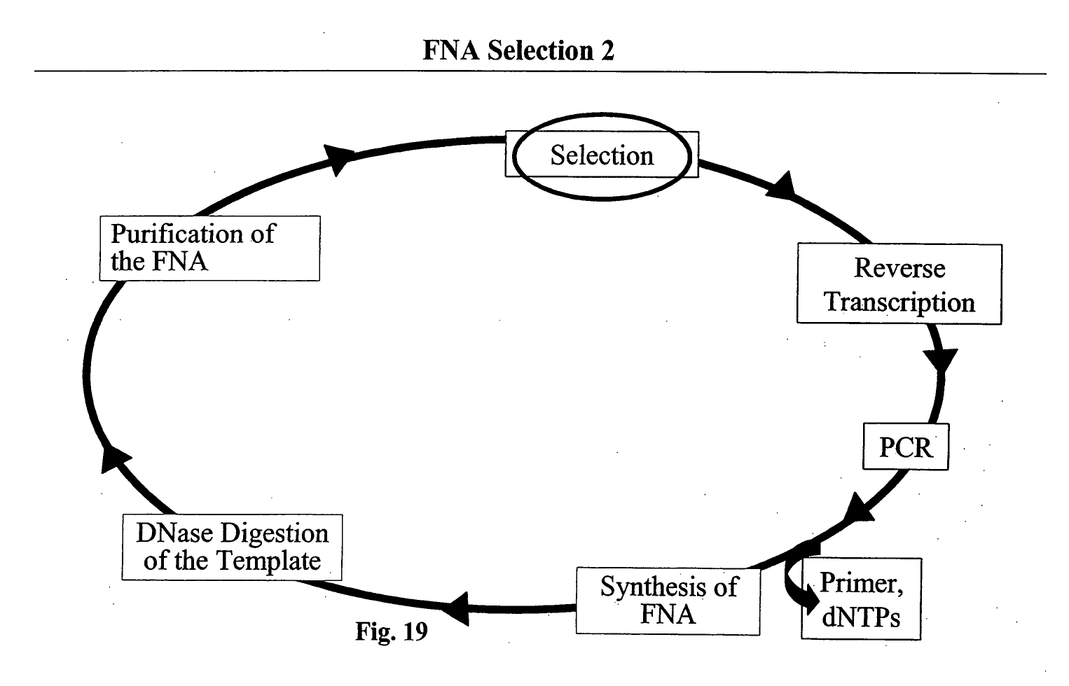

FIGS. 19-25 shows an alternative approach for the amplification of FNAs using the method of the present invention for the synthesis of nucleic acids, in particular also how such method can be incorporated into an in vitro selection process. In contrast to the embodiment depicted in FIGS. 5 to 12, there is no in vitro transcription in this embodiment.

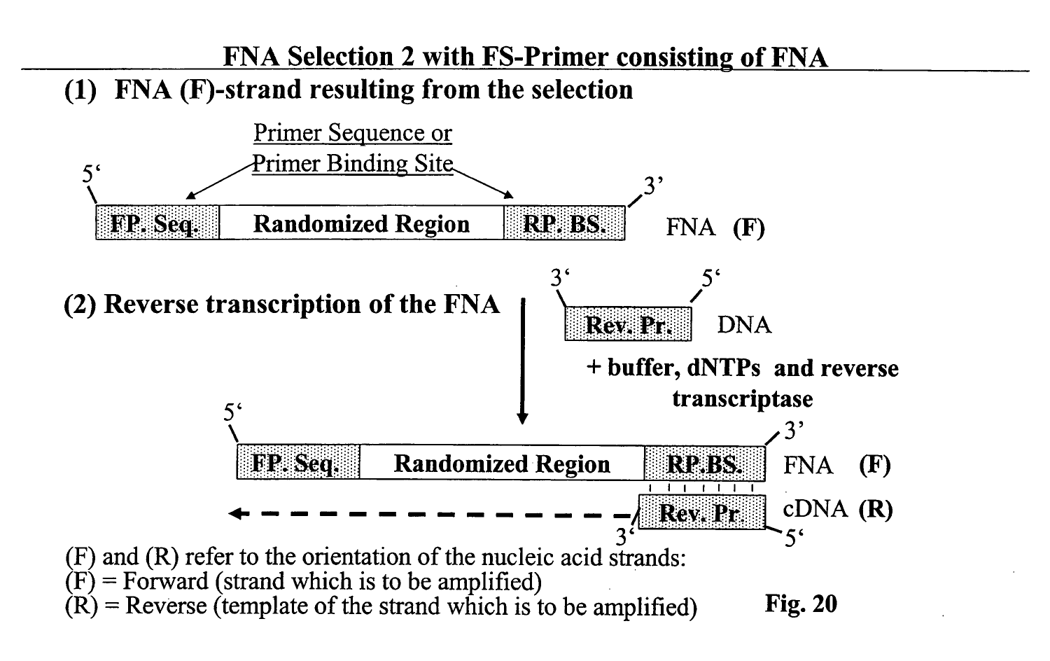

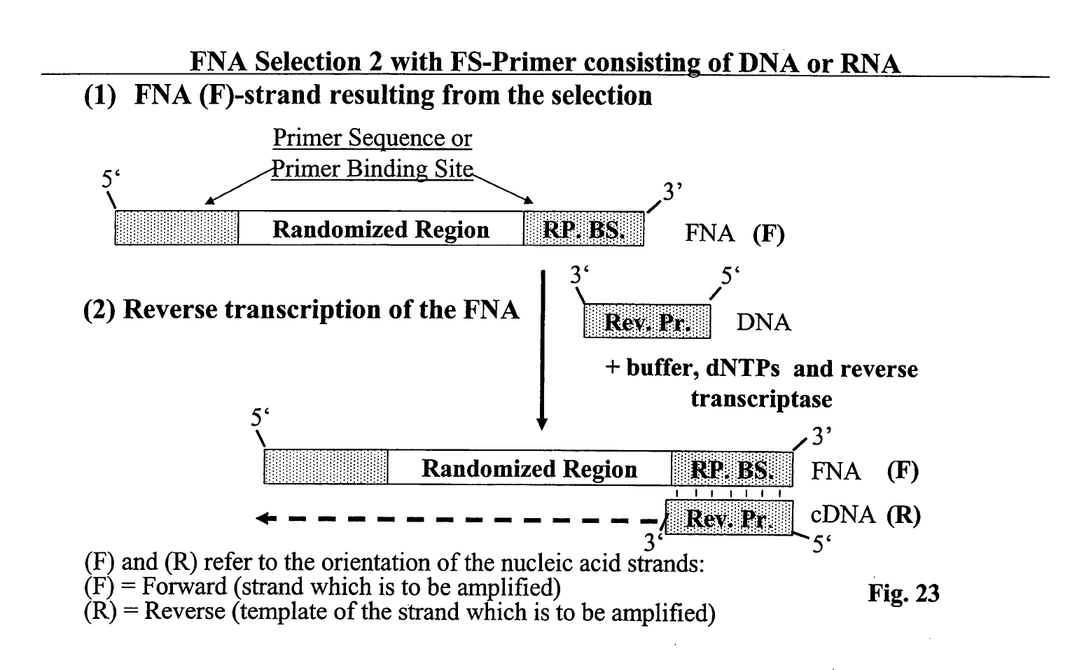

FIG. 19 shows an overview of the cyclic process comprising the following steps: selection, reverse transcription, PCR amplification, FNA synthesis and FNA isolation. FIGS. 20 to 22 show the method using a forward synthesis primer consisting of FNA, and FIGS. 23 to 25 show the method using a forward synthesis primer consisting of DNA or RNA.

In principle, it is to be acknowledged that the method is performed in a manner similar to the one described in FIGS. 5 to 12, whereby skipping the transcription step results in some changes as explained in more detail in the following.

FIG. 20 shows the synthesis of cDNA by means of reverse transcription starting from selected 2′-F-oligonucleotides, i.e. the starting nucleic acid, whereby a reverse primer is used hybridizing to the reverse primer binding site of the nucleic acid to be amplified. In contrast to the method shown in FIGS. 5 to 8, here the primer is lacking the region which acts as RNA polymerase promoter. A cDNA strand is obtained as reaction product of the reverse transcriptase which is complementary to the FNA strand used.

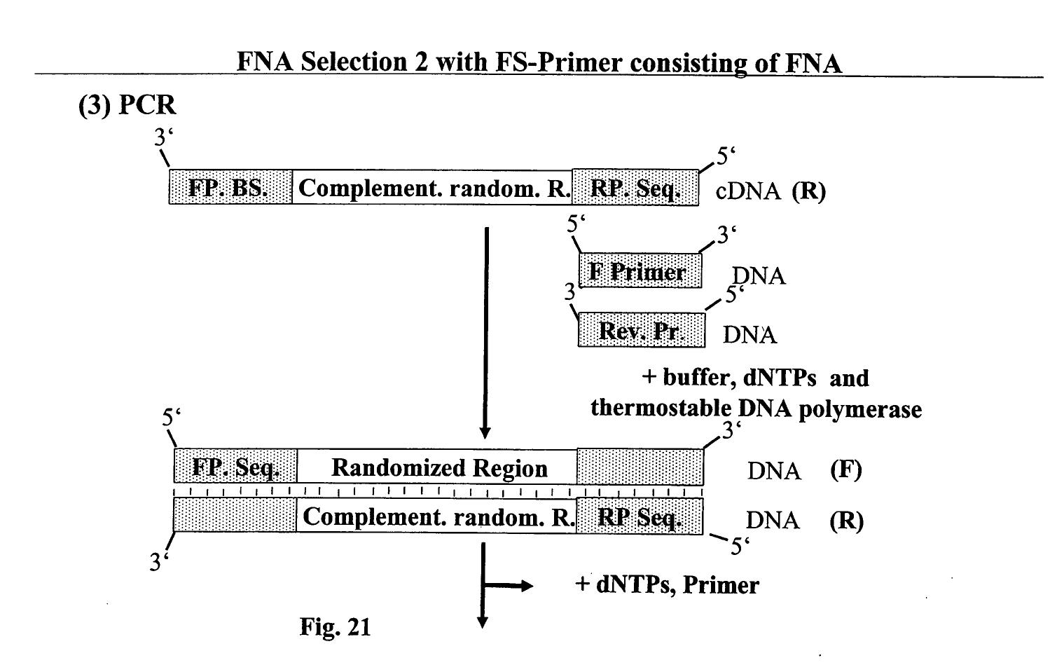

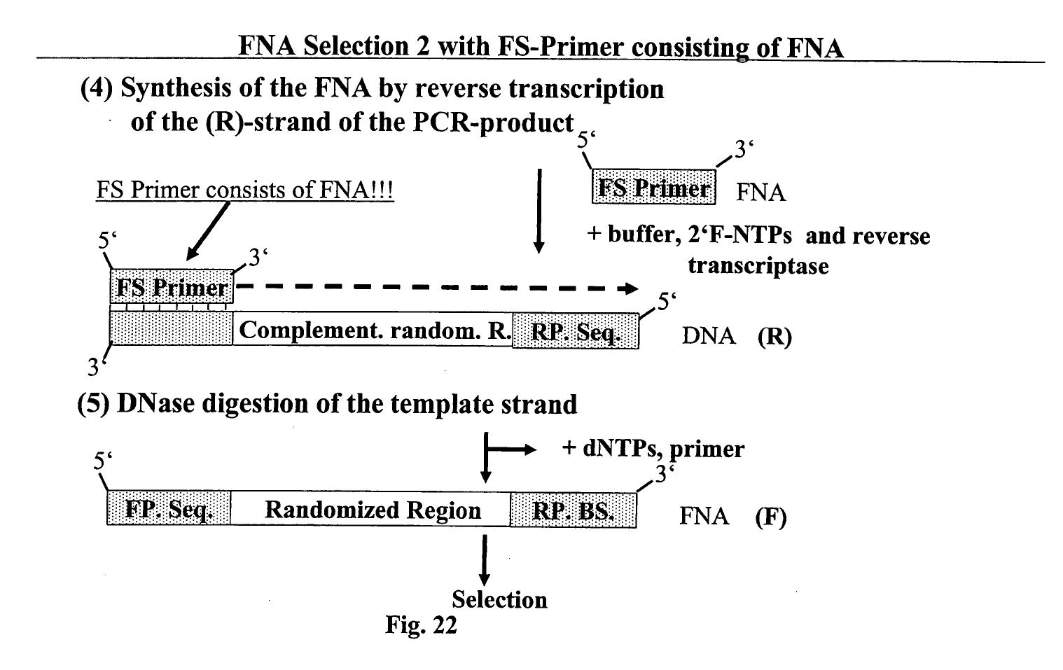

The polymerase chain reaction depicted in FIG. 21 which, apart from the second strand synthesis, also provides for amplification, also includes the reverse primer as well as a forward primer which is, in principle, designed similar to the one used in connection with the polymerase chain reaction of the method according to FIG. 6. The cDNA, i.e. the reaction product of the reverse transcriptase is thus amplified. The excess primer as well as nucleotides used in the PCR reaction are subsequently removed, for example by a molecular sieve. The synthesis of FNA nucleic acid is, as depicted in FIG. 22, subsequently carried out by reverse transcriptase of the (R)-strand of the PCR product consisting of DNA, whereby this R-strand is essentially complementary to the nucleic acid to be amplified and depicted in step 1 of FIG. 20, i.e. the starting nucleic acid. The R-strand of the PCR serves as template. The synthesis of the FNA occurs by means of a reverse transcriptase, an FNA primer and 2′-F-NTPs. The template strand is digested by DNase and the fragments and nucleotide, respectively, are removed. The thus obtained reaction product corresponds to the amplified nucleic acid and can be introduced into a subsequent selection round accordingly.

FIG. 23 shows the synthesis of cDNA by means of reverse transcriptase starting from the selected 2′-F-oligonucleotides, i.e. the starting nucleic acid. A cDNA strand is obtained as reaction product. In contrast to the procedure shown in FIG. 9 which still comprises a transcription step, in this embodiment of the invention it is envisaged that the reverse primer consists of the partial region only, which hybridizes to the reverse primer binding site (FIG. 14). A region which contains a promoter for a RNA polymerase, is typically absent.

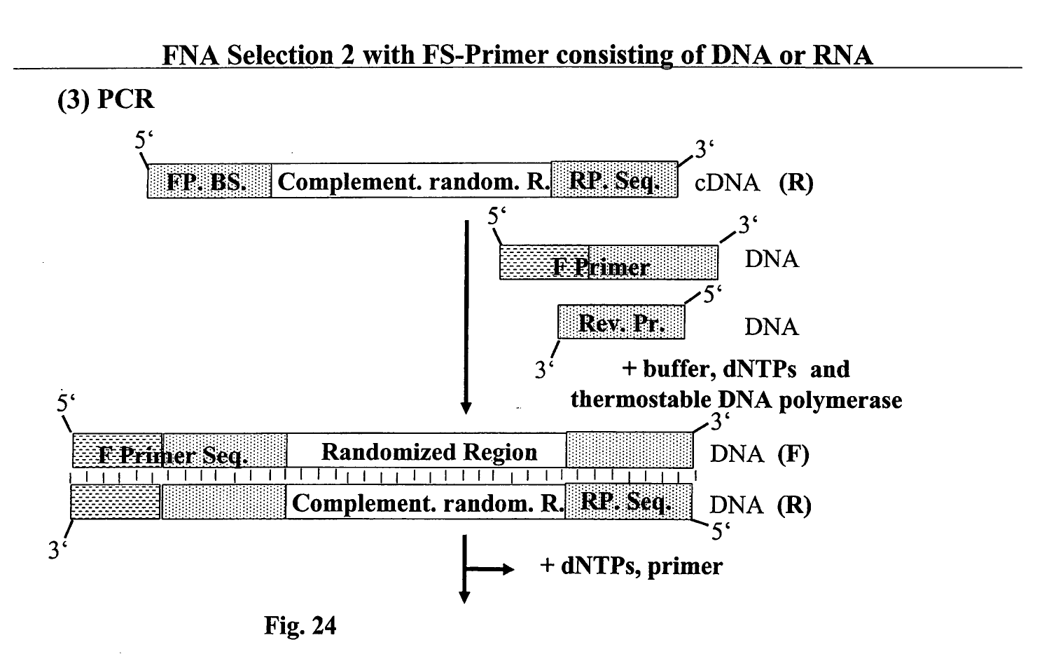

Two primers are used in the following polymerase chain reaction (FIG. 24) which is for the amplification of the cDNA, namely a forward primer and the reverse primer. The forward primer has at its 5′-end a further partial region which corresponds to the forward synthesis primer which, later, is used in the synthesis step using reverse transcriptase. The excess dNTPs as well as the primer are removed following the PCR, for example by molecular sieve.

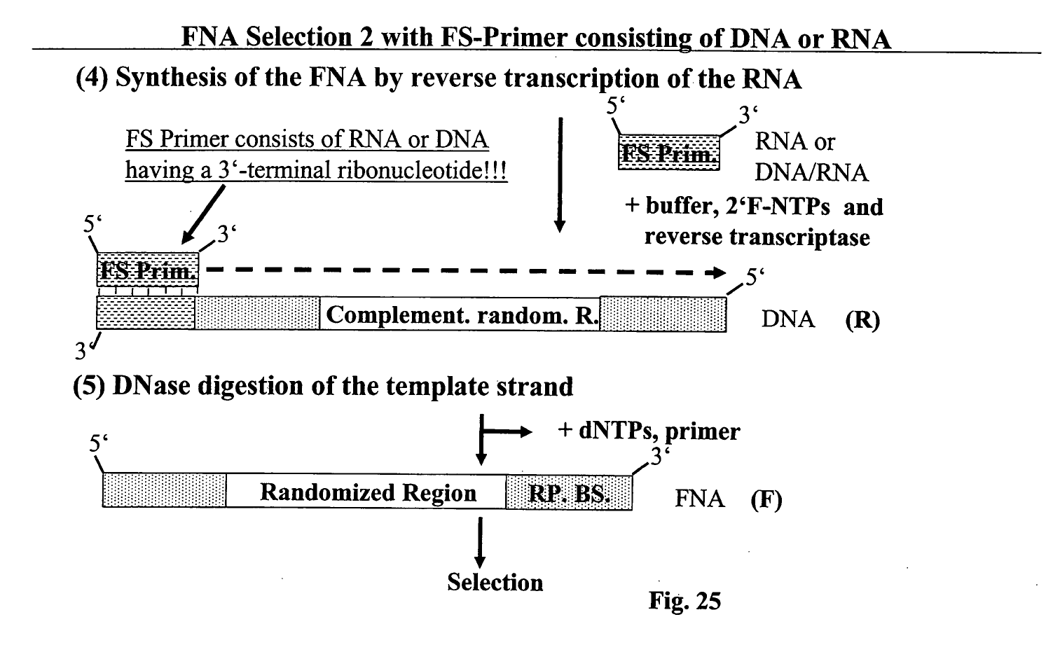

FIG. 25 finally shows the synthesis of the FNA by reverse transcription. In this embodiment, the forward synthesis primer will consist of RNA or a mixture of DNA/RNA, whereby the 3′-end of the forward synthesis primer must exhibit one ribonucleotide. The primer must, least at the 3′-end, be identical to the further partial region of the forward primer. After the reverse transcription reaction an alkaline hydrolysis or an RNase digestion is performed in order to remove the RNA components of the double-strand and to, thus, obtain the single-stranded FNA. If the forward synthesis primer consists of DNA, it is to be removed by a separate treatment, for example by DNase treatment, in order to obtain an amplification product which corresponds to the nucleic acid molecule shown in step 1 of FIG. 23 which is to be amplified.

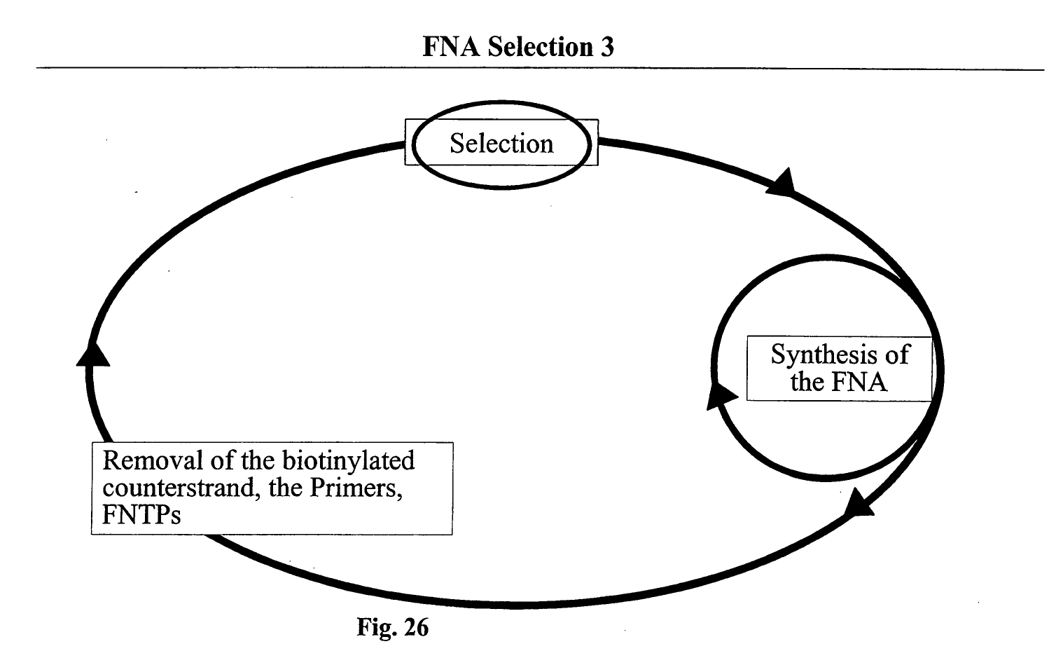

FIG. 26 to 29 show a further amplification method for modified oligonucleotide, in particular 2′-fluoro oligonucleotides, using the method of the invention for the synthesis of nucleic acid, whereby, essentially, a reverse transcriptase activity and a thermocycling are realized.

FIG. 26 shows the reaction sequence for the amplification of 2′-fluoro oligonucleotides within an in vitro selection process. It only consists of the repeated copying of the FNA by means of reverse transcriptase, preferably a thermostable reverse transcriptase, such as, for example, the DNA polymerase of Thermus thermophilus. Alternatively, also a device can be provided, where the reverse transcriptase is immobilized, whereby the FNA strands are denatured, and which specially separates therefrom primer-dependent synthesis of new strands at low temperature. This can, for example, be realized by a system consisting of two independent incubation vessels which are linked to each other. In such system the synthesized FNA, i.e. both the product as well as the starting material, is cycled in a buffer in the presence of FNTPs and primers, namely the forward synthesis primer and the biotinylated reverse primer, and, respectively, moved between the vessels. In the first vessel the temperature of which is the reaction temperature (for example 42-51° C.) the immobilized reverse transcriptase is contained which copies the present FNA molecules. The resulting double strands are subsequently separated from each other at high temperature of 95-99° C. in the second vessel and cooled down on their way to the first reaction vessel. As an alternative to immobilization, also a molecular sieve can be mounted at the exit of the reaction vessel in which the enzyme is contained, which retains the reverse transcriptase.

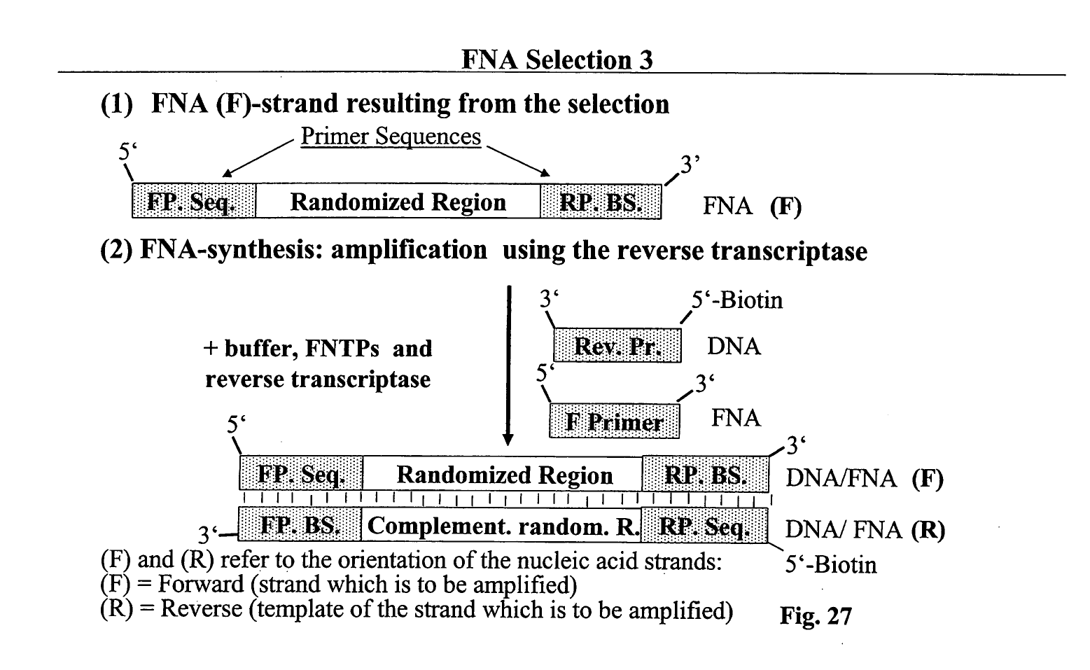

FIG. 27 shows the amplification of FNA by thermocycling with FNTPs, a reverse transcriptase and a suitable forward primer consisting of FNA, and a reverse primer consisting of DNA. The reaction contains, apart from the primer, also 2′-F-NTPs as well as a reverse transcriptase such as, for example, Superscript II of Invitrogen. The reverse primer consists of DNA and can, due to its complementarity to the reverse primer binding site of the nucleic acid to be amplified, i.e. the starting nucleic acid, hybridize thereto. The reverse primer has, in the present case, a label at the 5′-end which allows immobilization to a surface. In the present case the label is biotin which may interact with streptavidine immobilized to a surface, and may thus immobilize the primer and the molecule and moiety associated therewith to the surface, respectively. A double-stranded nucleic acid molecule is the result of the reverse transcription, whereby a strand which is also referred to herein as (F)-strand, corresponds to the nucleic acid molecule to be amplified, and the second strand is complementary thereto and is referred to as (R)-strand. The FNA primer hybridizes to the forward primer binding site of the (R)-strand of the first and subsequently copied (F)-strand, respectively, which consists of FNA and acts as template strand.

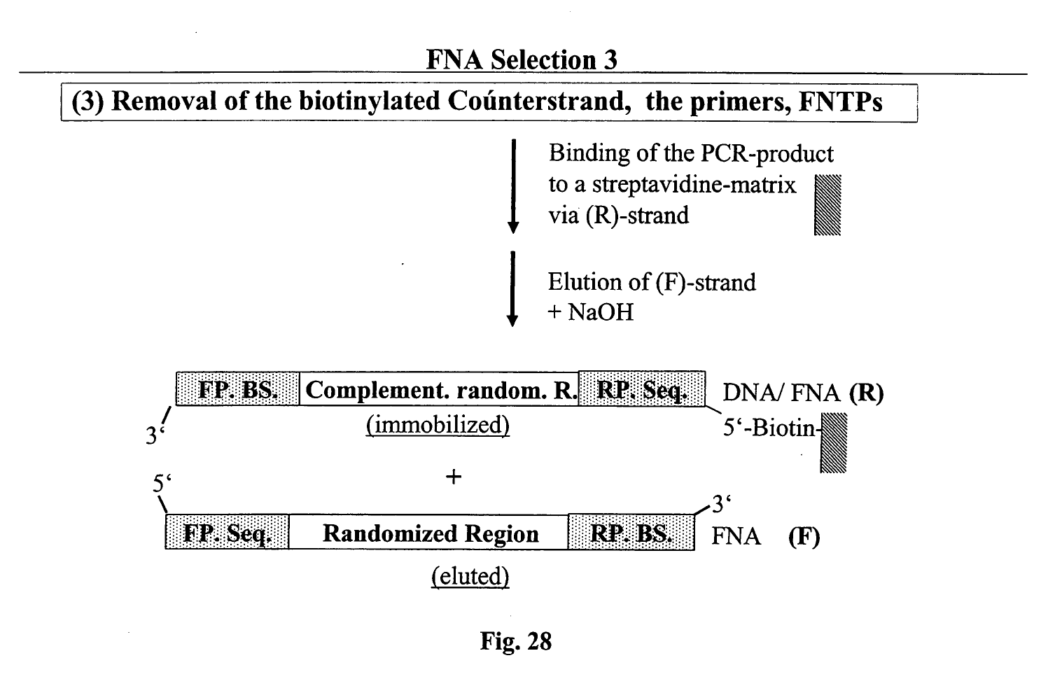

The thus obtained double-stranded product is immobilized to a streptavidine comprising matrix by means of the label on the complementary strand as shown in FIG. 28. As an alternative, neutravidine can, for example, be used instead of streptavidine. Suitable surfaces are, for example, agaroses, the surface of vessels or the surface of beads, in particular magnetic beads. The non-incorporated forward primer and NTPs may thus be washed away from the reaction. For releasing the desired amplified acid molecule which corresponds to the one shown in FIG. 27, partial step (l), an elution is performed by, for example, NaOH of the desired amplification product, i.e. the strands corresponding to the nucleic acid shown in partial step (l) of FIG. 27.

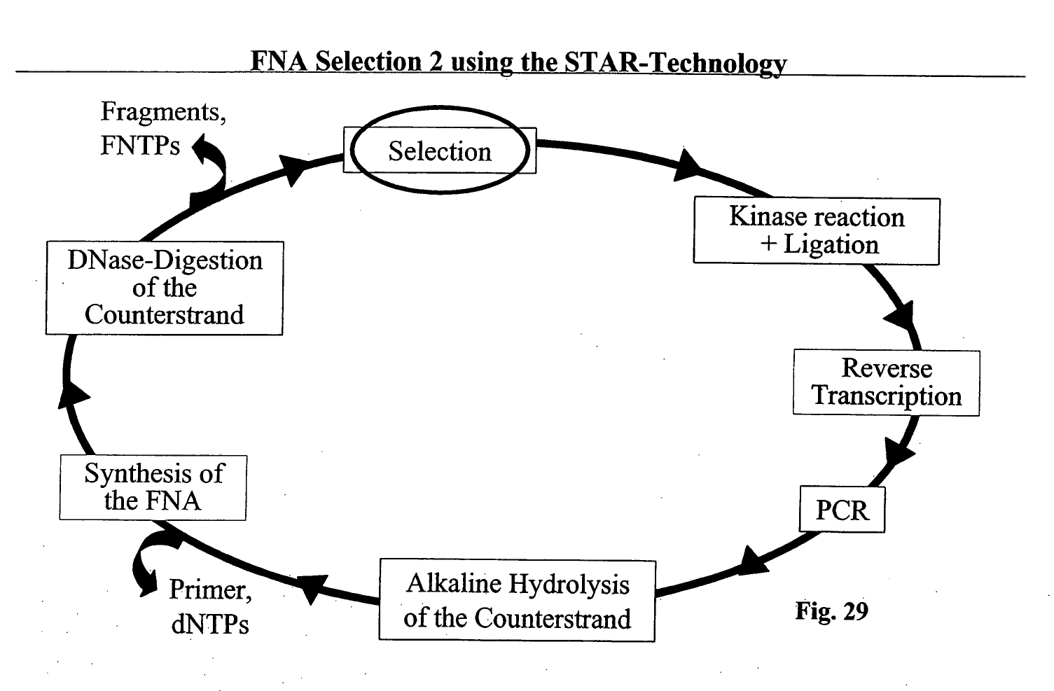

FIGS. 29 to 34 show the amplification of FNA sequences having only a few, i.e. typically four to six known nucleotides at each end of the randomized region by using the STAR-technology described in FIGS. 13 to 18, and is a further embodiment thereof, whereby in this embodiment the in vitro transcription step is not performed. The particular steps consist of a kinase reaction and ligation, reverse transcription, polymerase chain reaction, alkaline cleavage of the counterstrand, synthesis of the FNA as well as DNase digestion of the counterstrand in order to obtain a nucleic acid which corresponds in its basis design to the starting nucleic acid as used in the selection.

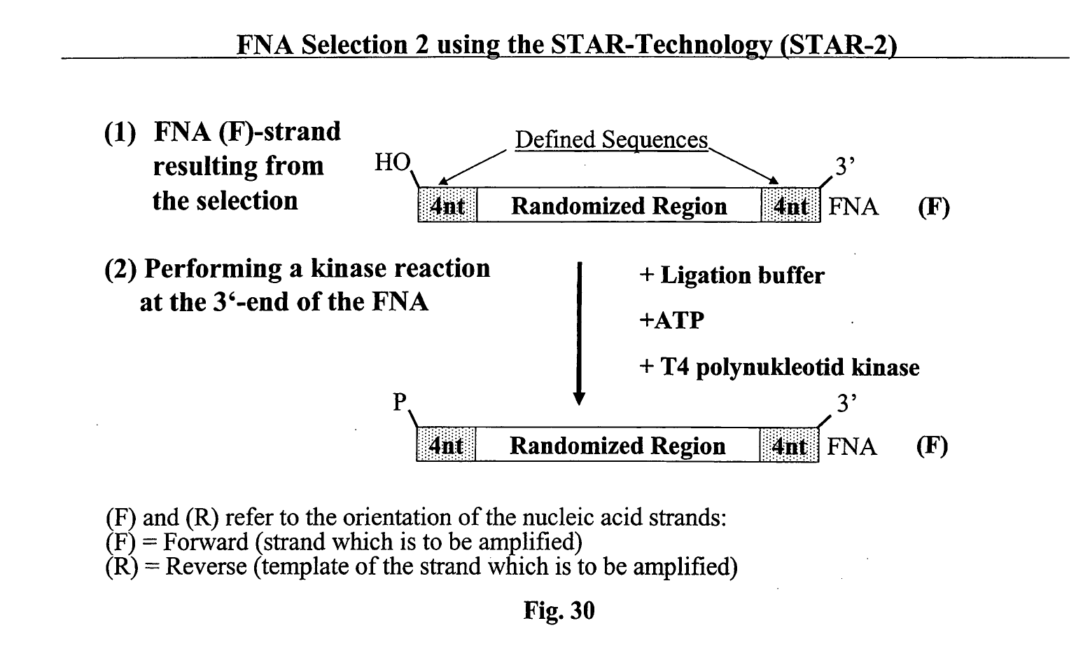

FIG. 30 (1) shows again the design of the nucleic acids used as starting population in connection with the in vitro selection, as it is also described in FIG. 14 (1). The further shown step of a 5′-phosphorylation of this starting nucleic acid corresponds to the method described in connection with FIG. 14.

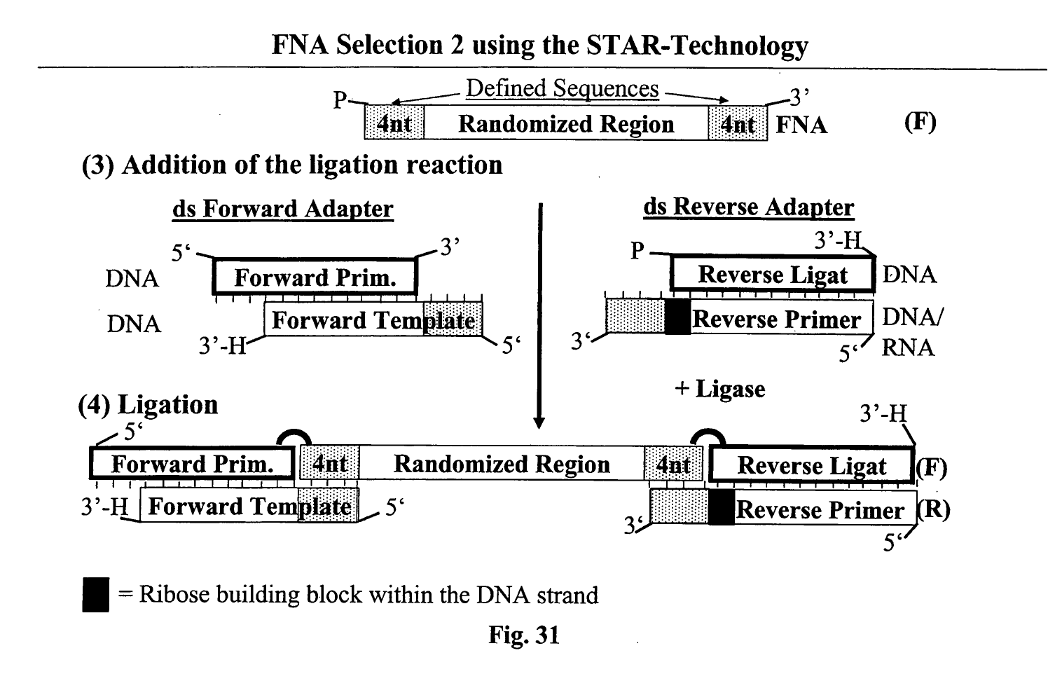

The ligation method shown in FIG. 31 shows the ligation of the first and the second adapter molecule which each consist of a first and a second nucleic acid strand. In the present case, both adapter molecules consists of DNA, whereby the second strand of the second adapter molecule comprises a cleavage site in the form of the ribonucleotide which allows for a cleavage of the nucleic acid strand using alkali and optionally heat. The design of the first and the second adapter molecule and the corresponding nucleic acid strands, respectively, otherwise corresponds to the design described in connection with FIG. 15. In a way similar to the method described in connection with FIG. 15 a DNA ligase, preferably a T4-DNA-ligase, can be used as the ligase. The nucleic acid strands are, similar to the ones of FIG. 15, preferably blocked at the 3′-end, for example by the terminal nucleotides being 2′-3′-dideoxynucleotides. The cleavage site is arranged such that it results in a dissociation of the second nucleic acid strand of the second adapter molecule at the 5′-end of that partial region of the nucleic acid strand of the second adapter molecule which hybridizes to the second constant sequence of the starting nucleic acid. The product obtained from this reaction consists, first, of the starting nucleic acid at the 5′-end of which the first nucleic acid strand of the first adapter molecule is ligated, and at the 3′-end of which the first nucleic acid strand of the second adapter molecule is ligated, whereby the second nucleic acid strand of the first adapter molecule is hybridized to the first nucleic acid strand of the first adapter molecule now ligated to the starting nucleic acid, as well as the first constant partial sequence thereof, and the second nucleic acid strand of the second adapter molecule is hybridized to the first nucleic acid strand of the second adapter molecule and the second constant partial sequence of the starting nucleic acid, whereby in this double-stranded nucleic acid the randomized region of the starting nucleic acid is not contained.

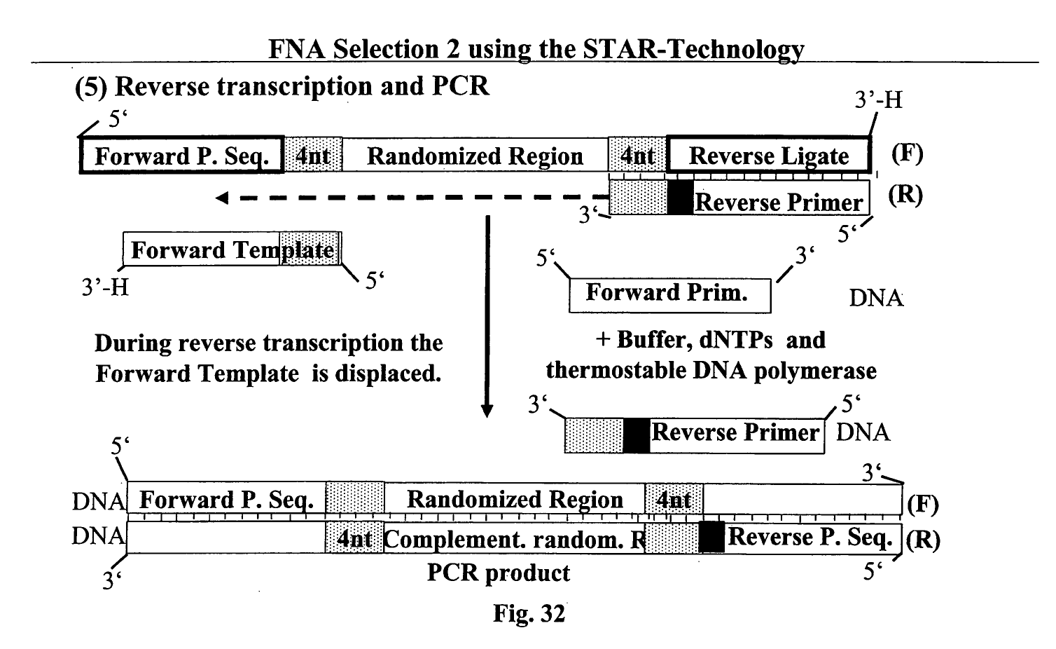

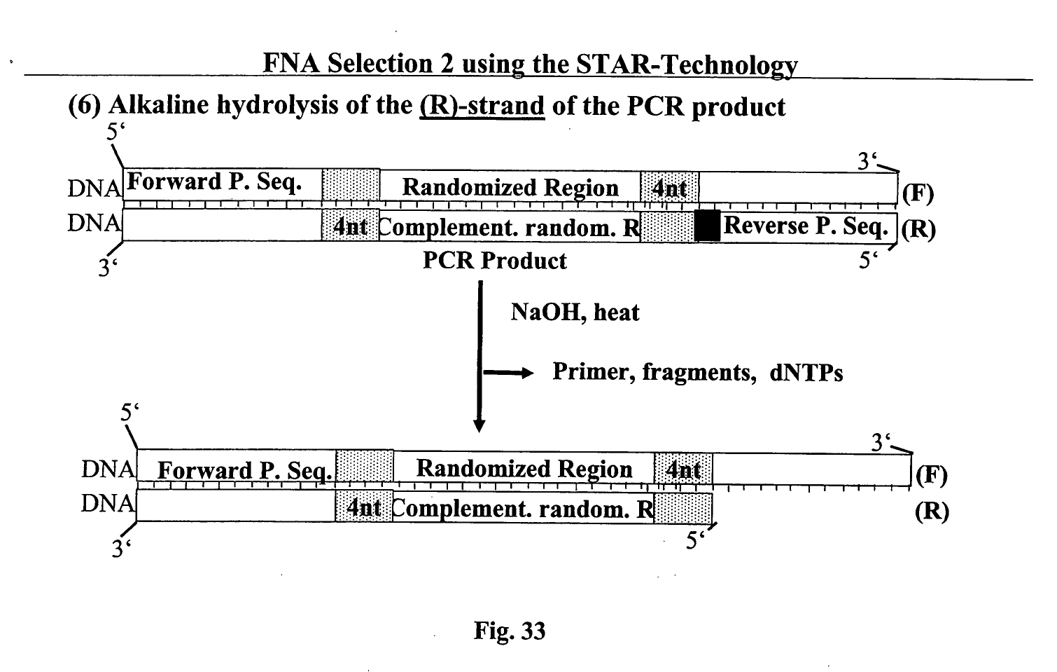

As depicted in FIG. 32, starting from the ligation product, i.e. the starting nucleic acid which is ligated with the first nucleic acid strands of the first and the second adapter molecule, a cDNA is synthesized by means as reverse transcriptase using the reverse primer. The cDNA strand comprises the cleavage site due to the use of the second nucleic acid strand of the adapter molecule. The reverse transcription is followed by an amplification reaction, in particular a polymerase chain reaction, whereby the first nucleic acid strand of the first adapter molecule as well as the second nucleic acid strand of the second adapter molecule are used as primers, whereby a suitable polymerase, in particular a DNA polymerase and more preferably a thermostable DNA polymerase is added to the reaction which subsequently incorporates the nucleotides. The second strand of the first adapter molecule is removed from the ligation product during reverse transcription. At the end of the amplification reaction a double-stranded nucleic acid is obtained, whereby such nucleic acid corresponds in its design to the ligation product and the strand complementary thereto. Subsequently, the part of the complementary strand is cleaved off by alkali treatment and heat treatment which is not complementary to the second constant sequence at the 3′-end of the starting nucleic acid. The primer as well as the dNTPs are then removed (FIG. 33).

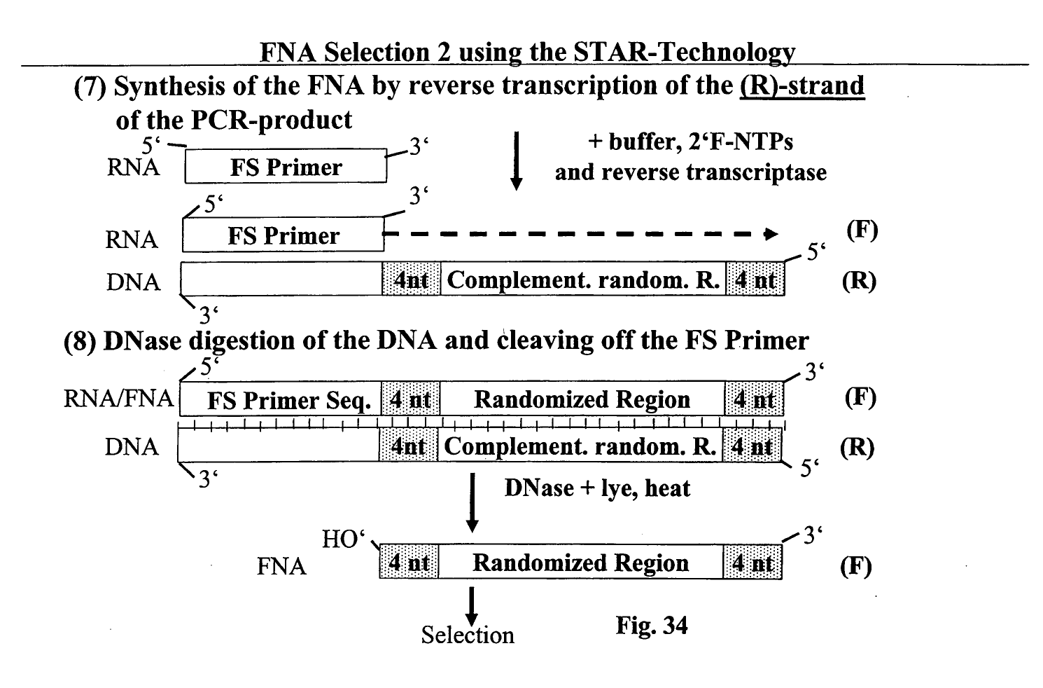

FIG. 34 shows the FNA synthesis with a forward synthesis primer using RNA, 2′-F-NTPs and a reverse transcriptase. The single-stranded nucleic acid obtained in the preceding step serves as template strand from which the part of the second nucleic acid strand of the second adapter molecule is removed which does not hybridize with the second constant partial region. The thus synthesized strand comprises the sequence of the forward synthesis primer consisting of RNA, the first constant partial sequence, the randomized region as well as the second partial sequence of the starting nucleic acid. This strand is present as a complex with the template strand which consists of DNA and which can be removed by subsequent DNase treatment. The sequence of the forward synthesis primer is subsequently removed by treatment with RNase or alkali at increased temperature or a combination of both, in order to obtain a nucleic acid which essentially corresponds to the starting nucleic acid.

FIGS. 35 to 38 show the method of the invention for the amplification of single-stranded DNA (ssDNA), incorporated into the context of an in vitro selection. The method starts with an amplification of the ssDNA by PCR serving as starting nucleic acid whereby, under the proviso that there is enough material, it can be replaced by a second strand synthesis, followed by an amplification of the counterstrand which is effected by an in vitro transcription. The transcription product subsequently serves as template for the synthesis of the ssDNA by means of a reverse transcriptase, a DNA primer and dNTPs. The template of the reverse transcription can be disassembled in its mononucleosides (2′-3′-cyclic phosphate, 5′-OH) by alkaline hydrolysis. These mononucleosides can easily be removed from the DNA strand by, for example, filtration with a molecular sieve, alcoholic precipitation with linear polyacrylamid and ammonium acetate.

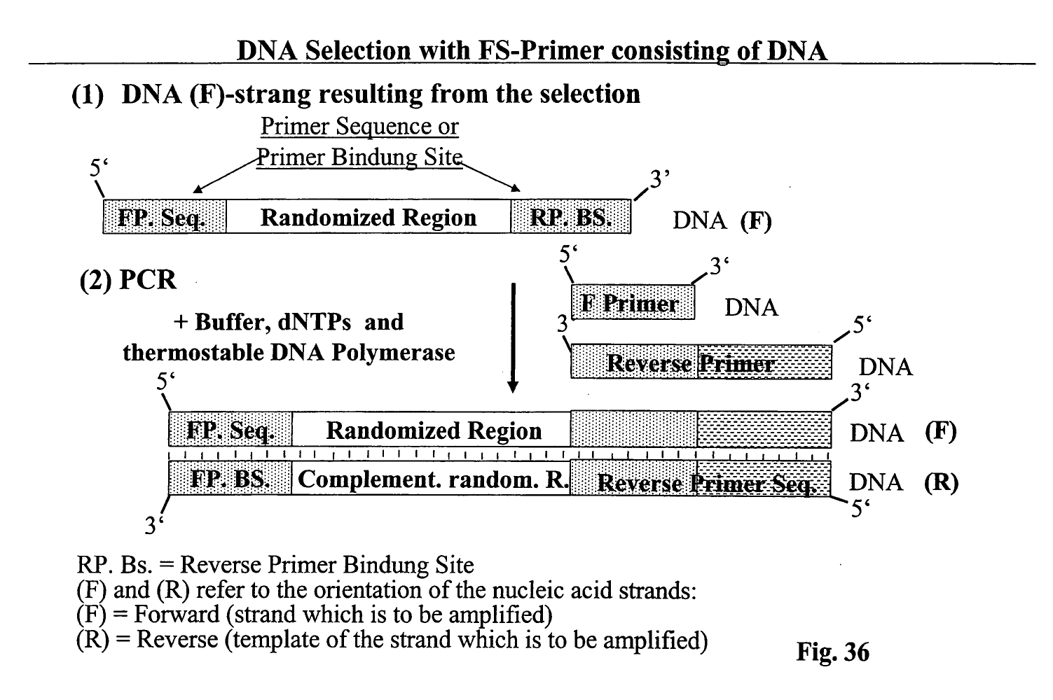

FIG. 36 shows the PCR amplification of the ssDNA to be amplified (forward strand). The reverse primer contains a 5′-overhang portion containing the T7-RNA polymerase promoter. The PCR product consists of a forward and a reverse strand.

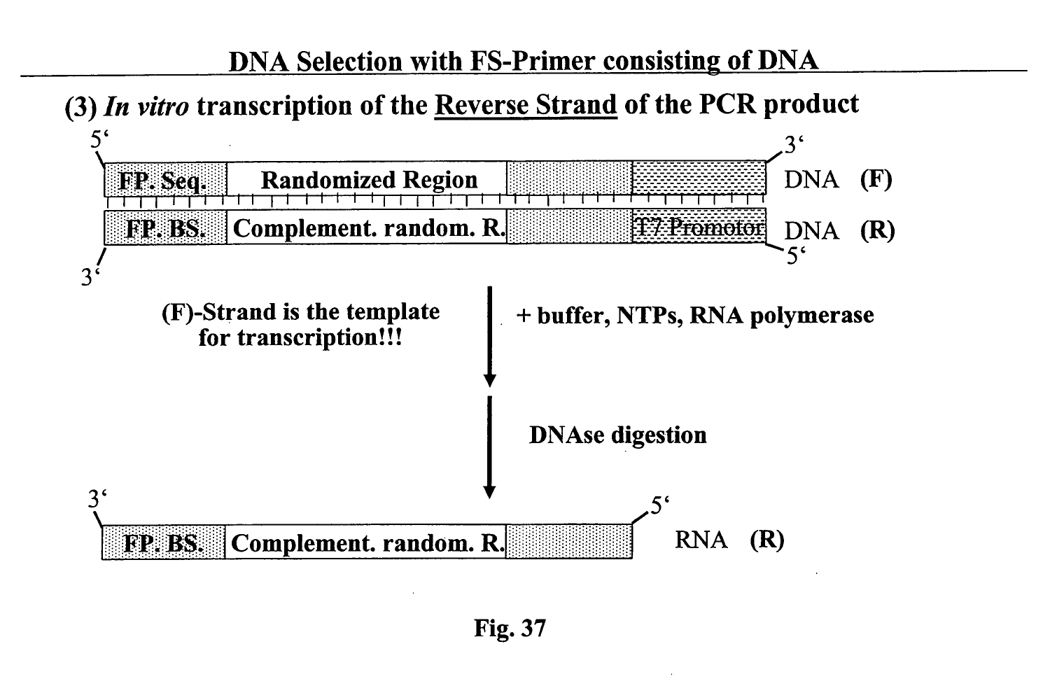

FIG. 37 shows the in vitro transcription of the reverse strand, whereby the forward strand acts as a template for the RNA synthesis. The promoter region is not transcribed. Subsequently, the PCR product and excess primers are digested.

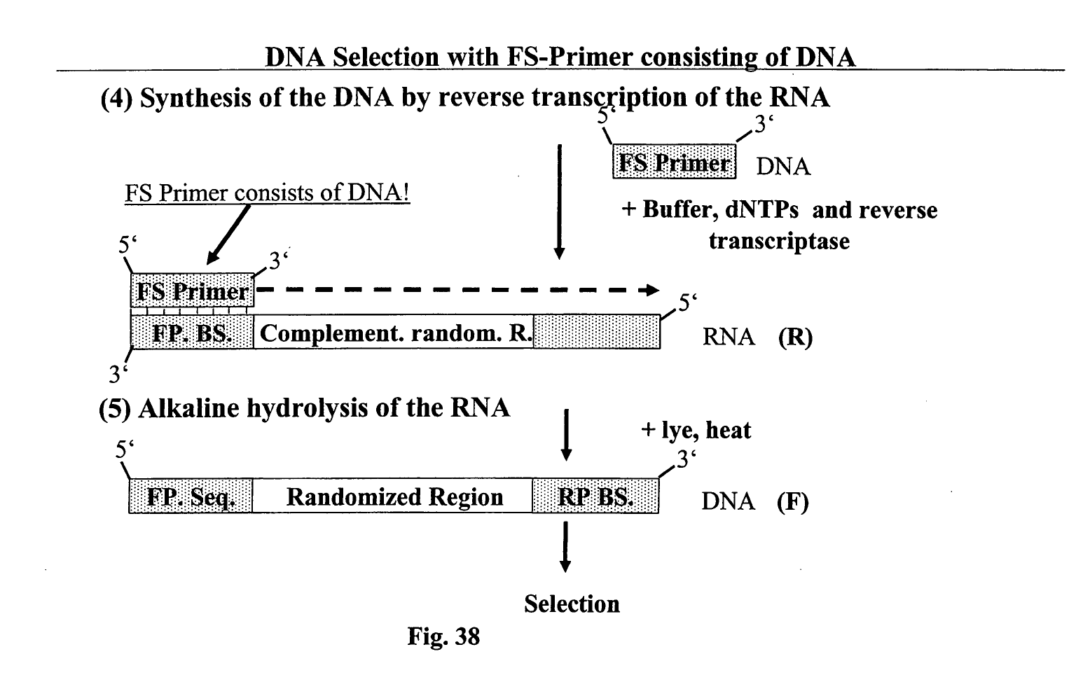

FIG. 38 shows the synthesis of the single-stranded DNA at the RNA template by means of a reverse transcriptase, a suitable DNA primer and dNTPs. A template is subsequently hydrolyzed by lye and heat, so that single-stranded DNA has to be purified from nucleotides only, but not from a counterstrand of similar or identical length.

FIG. 39 shows the experiment of performing an amplification cycle with the steps of FNA selection 1. The starting material was a RNA library. Such RNA library was then transcribed into an FNA library (having a DNA primer at the 5′-end) by reverse transcriptase with a DNA primer and FNTPs. After reverse transcription with dNTPs and an overhanging reverse primer (the T7-RNA-polymerase promoter region forms an overhang) a PCR was performed. The PCR product was subsequently used again as starting material for in vitro transcription. By doing so the cycle was completed. The reactions are described in more detail in example 1 under the heading “FNA library for performing the amplification cycle”.

FIG. 40 shows the DNase stability of an FNA library which was previously prepared by means of reverse transcriptase from RNA. The DNA/RNA primer was removed from the FNA by alkaline hydrolysis prior to DNase incubation. Upon incubation for the same period of time FNA is significantly more than 100× stable to DNase as a similarly long DNA which had been prepared in the same manner. The reaction is described in example 1 under the heading “Synthesis of the FNA library for stability studies” and in example 2 under the heading “DNase I stability”.

FIG. 41 shows the stability towards RNase T1 and RNase I of an FNA library prepared by reverse transcriptase, and of a 2′-F-pyrimidine RNA prepared by in vitro transcription. The reaction is described in example 3 “RNase stability”.

FIG. 42 shows the stability in human serum of the FNA library prepared by reverse transcriptase and of an RNA prepared by in vitro transcription. The reaction is described in example 4 under the heading “RNase stability”.

FIG. 43 shows the selection course of the in vitro selection described in example 1.

### EXAMPLE 1

### Use of FNA for the Selection of Protein-Binding Nucleic Acids for Validation of the Procedure

For the herein described experiments the itemized materials were used whereby a detailed specification of the suppliers of itemized substances, solutions and enzyme is cited in the corresponding text passage. Unless otherwise indicated the reagents were purchased from Merck (Darmstadt, Germany). In all cases LiChromosolv water from Merck (Darmstadt, Germany) was used.

The protein (basic, pI of 9; molecular mass of 9 kDa, do not bind to nucleic acids per se) used for in vitro selection was labeled according to the manufacturer's instructions with a biotin linker at least one of the accessible amino groups of the amino acids using the Biotinylation-Kit “EZ-Link Sulfo-NHS-LC-LC-Biotin” (Pierce, Rockford, USA). Thus, the separation of unbound nucleic acids using the biotin-streptavidine- or biotin-neutravidine-linkage can be realized. For this purpose the selection matrices neutravidine agarose and “UltraLink Plus” immobilized streptavidine gel (both purchased from Pierce, Rockford, USA) was used. Excess linker was removed according to the manufacturer's instructions with a molecular sieve, the filter device “YM-3” (molecular cut-off limit of MW 3000, Amicon/Milliore, Bedford, USA).

The used oligonucleotides such as primers and die initial DNA library were synthesized by standard phosphoramidite chemistry at NOXXON Pharma AG. The sequences can be found in Example 6.

### Synthesis of the FNA Library

### Synthesis of the Template for the FNA-Synthesis: Fill-In Reaction+In Vitro Transcription

At first 3 nmol of a synthetic DNA library BSA-1C initial library was converted into double-stranded DNA by a Fill-in-reaction using the BSA 1C-Reverse Primer T7 and the Vent-(exo⁻)-DNA polymerase (NEB, Frankfurt a.M., Germany). By using this reaction with the BSA 1C-Reverse Primer T7 the T7 RNA polymerase promoter was incorporated.

### Fill-In-Reaction

| Component | final concentration |
|---|---|
| 10x buffer (NEB) | 1x |
| Betain | 0.5 M |
| dNTPs (Larova, Teltow, Germany) | 0.5 mM |
| BSA-1C Reverse Primer T7 | 6 μM |
| BSA-1C initial library | 2 μM |
| Vent-(exo⁻)-DNA polymerase (NEB) | 10 U/l00 μl_{reaction volume} |

At first the batch without enzyme was denatured at 95° C. for 10 min. and cooled for 5 minutes on ice. Then the enzyme was added and the batch was incubated for two hours at 63° C.

Afterwards the dsDNA was desalted by ethanol precipitation (20 μg GlycoBlue (Ambion) and 2.5 vol. absolute ethanol, 30 min at −80° C.; centrifugation at 13200 rpm (16100 g) for 30 minutes at 4° C.; the pellet was once washed with 70% ethanol).

The dsDNA library was used as template for in vitro transcription.

### In Vitro Transcription

| Component | final concentration |
|---|---|
| Transcription buffer (80 mM HEPES/KOH, | 1x |
| pH 7.5, 22 mM MgCl₂; 1 mM Spermidin) | |
| DTT | 10 mM |
| NTPs (Larova, Teltow, Germany) | 4.0 mM |
| RNaseOUT (Invitrogen, Carlsbad, USA) | 1 μl/100 μl_{reaction volume} |
| Fill-in reaction product | 1 μM |
| T7 RNA polymerase | 50 U/100 μl_{reaction volume} |
| (Stratagene, La Jolla, USA) | |

Incubation: 2 to 12 hours at 37° C.

Subsequent to the in vitro transcription the remaining dsDNA template was digested with 20 units DNAse I (Sigma) for 20 min at 37° C. After addition of loading buffer (7M urea, xylene cyanol, bromphenol blue) the batch was denatured and gel-purified (10% denaturing polyacrylamide gel). The band of the transcript was detected by UV-Shadowing, cut off from the gel and eluted by the Crush-and-Soak method. The resultant eluate was precipitated in ethanol, the pellet washed once with ice-cold 70% ethanol and resuspended in water.

The RNA library was used as template for FNA-synthesis using a reverse transcriptase.

### FNA-Synthesis and Labeling of the FNA

### FNA-Synthesis

| | final |
|---|---|
| Component | concentration |
| RNA library | 1 μM |
| BSA-1C FS Primer 3rG | 5 μM |
| Q-solution (Qiagen, Hilden, Germany) | 1x |
| 5 min at 95° C., 5 min on ice | |
| 1st strand buffer (Invitrogen, Carlsbad, USA) | 1x |
| DTT (Invitrogen, Carlsbad, USA) | 10 mM |
| FNTPs (TriLink Biotech, San Diego, USA) | 0.5 mM |

Superscript II (Invitrogen, Carlsbad, USA)

Temperature program:

| | |
|---|---|
| 1. 51° C. | 20 min |
| 2. 54° C. | 10 min |

Subsequent to the FNA-synthesis the template-strand (RNA) was hydrolyzed under alkaline conditions.

### Alkaline Hydrolysis of the Template Strand

| Component | final concentration |
|---|---|
| Reaction product of the FNA-synthesis | total |
| NaOH | 0.3 M |
| 10 min at 95° C., 5 min on ice | |
| NaOAc | 0.1 M |
| HCl for neutralization | 0.3 M |

Afterwards the FNA library was desalted by ethanol precipitation (20 μg GlycoBlue (Ambion) and 2.5 Vol. absolute ethanol, 30 min at −80° C.; centrifugation at 13200 rpm (16100 g) for 30 minutes at 4° C.; the pellet was once washed with 70% ethanol). After addition of loading buffer (7M urea, xylene cyanol, bromphenol blue) the batch was denatured and gel-purified (10% denaturing polyacrylamide gel). The band of the FNA was detected by UV-Shadowing, cut off from the gel and eluted by the Crush-and-Soak method. The resultant eluate was precipitated in ethanol, the pellet washed once with ice-cold 70% ethanol and resuspended in water.

In order to detect the binding of the FNA library during the in vitro selection, the 5′-OH— group of the FNA library was radioactively labeled with ³²P by performing a kinase reaction.

### Labeling of the FNA

| Component | final concentration |
|---|---|
| FNA | 10 μM |
| “Forward”-buffer (Invitrogen) | 1x |
| γ-³²PATP | 1 μl/10 μl_{reaction volume} |
| T4 polynucleotide kinase (Invitrogen) | 1 U/μ_{reaction volume} |

The reaction was run at 37° C. for one hour and was stopped at 65° C. (10 min).

After addition of loading buffer (7M urea, xylene cyanol, bromphenol blue) the reaction was denatured and gel-purified (10% denaturing polyacrylamide gel). The FNA was detected by UV-Shadowing, cut off from the gel and eluted by the Crush-and-Soak method. The resultant eluate was precipitated in ethanol, the pellet washed once with ice-cold 70% ethanol and resuspended in water.

### Selection Steps

### Denaturation and Folding of the FNA

All non-enzymatic steps of the selection (except for the denaturation step) were carried out in selection buffer (20 mM Tris pH 7.4; 150 mM NaCl; 4 mM KCl; 1 mM MgCl₂ (all from Ambion, Austin, USA); 1 mM CaCl₂ (Merck, Darmstadt, Germany) and 0.1% Tween-20 (Roche Diagnostics, Mannheim, Germany). The denaturation step was carried out for five minutes at 95° C. in selection buffer. Subsequent to the denaturation step the FNA was cooled down to 37° C. for 15 minutes at 37° C.

### Binding Reaction

Subsequent to the folding, the FNA was at first incubated with the selection matrix (either with neutravidine agarose or with “UltraLink Plus” immobilized streptavidin, both from Pierce, USA) without protein at 37° C. for 30 minutes. This so-called “pre-selection” was done in order to remove potentially matrix binding molecules. After this incubation step the selection matrix was sedimented and the non-bound FNA in the supernatant was separated. An aliquot of the FNA was incubated with the biotinylated protein for one hour at 37° C. The other aliquot of the FNA was incubated without protein for one hour at 37° C. Subsequently, the biotin-binding selection matrix was added to the binding reaction. After incubation for 30 min at 37° C. the selection matrix including the complexes of protein and FNA bound thereto, was separated from the solution by centrifugation and washed with selection buffer.

### Elution of Bound FNA Molecules

The FNA which remained on the selection matrix after the washing step was eluted twice from the matrix material with 200 μl 8 M urea/10 mM EDTA (both from Ambion, Austin, USA), respectively. The first elution step was carried out for 15 minutes at 65° C., the second elution was done at 95° C. To the eluted FNA 400 μl of a mixture of phenol/(chloroform/isoamylalcohol) (1:(1:1/24)) (Applichem, Darmstadt, Germany) was added, the mixture was centrifuged for 5 min at 13000 rpm at room temperature. The aqueous phase (supernatent) was recovered, the phenolic phase was once re-extracted with 100 μl water, the aqueous phases were combined and shaken with 500 μl of a mixture of chloroform and isoamylyalcohol (24:1) (Applichem, Darmstadt, Germany), centrifuged for 5 min at 13000 rpm room temperature and the upper aqueous phase was separated.

Thereupon the aqueous phase was ethanol-precipitated (2.5 fold volume of absolute ethanol (Merck, Darmstadt, Germany), 0.3 M sodium acetate, pH 5.5 (Ambion, Austin, USA) and 1 μl glycogen (Roche Diagnostics, Mannheim, Germany)) for 30 min at −80° C. and centrifuged for 30 min at 14000 rpm (4° C.). The pellet was washed once with ice-cold 70% ethanol (Merck, Darmstadt, Germany).

### Amplification

### Synthesis of the cDNA

In order to amplify the FNA library via a PCR reaction the FNA was transcribed into DNA.

cDNA-Synthesis

| | final |
|---|---|
| Component | concentration |
| FNA resulting of selection | max. 0.5 μM |
| BSA-1C Reverse Primer T7 | 2.5 μM |
| Q-solution (Qiagen, Hilden, Germany) | 1x |
| 5 min at 95° C., 5 min on ice | |
| 1st strand buffer (Invitrogen, Carlsbad, USA) | 1x |
| DTT | 10 mM |
| dNTPs (Larova, Teltow, Germany) | 0.5 mM |
| Superscript II (Invitrogen, Carlsbad, USA) | 20 U/μl |

Temperature program:

| | |
|---|---|
| 1. 51° C. | 20 min |
| 2. 54° C. | 10 min |
| 3. 4° C. | infinite |

### PCR

| Component | final concentration |
|---|---|
| PCR buffer (NEB) | 1x |
| BSA-1C Forward-Primer | 5 μM |
| BSA-1C Reverse Primer T7 | 5 μM |
| dNTPs (Larova, Teltow, Germany) | 0.2 mM |
| vent-(exo⁻)-DNA polymerase (NEB) | 0.05 U/μl |
| cDNA | 0.1-0.5 μM |

Temperature program:

| | |
|---|---|
| 1. 95° C. | 4 min |
| 2. 95° C. | 1 min |
| 3. 68° C. | 1 min |
| 4. 72° C. | 1 min |
| 5. Go to 2 | 11x |
| 6. 72° C. | 6 min |
| 7. 4° C. | until stop |

In order to prepare the dsDNA for the following in vitro transcription, the DNA was ethanol-precipitated.

Subsequently, the RNA that had been synthesized by in vitro transcription, was transcribed into FNA. The RNA that was used as template and the BSA-1C FS-Primer 3rG were fragmented by alkaline hydrolysis and removed using a molecular sieve (YM-filter units, Amicon/Millipore, Bedford, USA). The FNA was radioactively labeled (as described). The thus enriched FNA library was used in the next selection round.

The products of the amplification steps to be performed during one selection round are depicted in FIG. 39. For this purpose aliquots of the products of the respective reactions were separated by denaturing polyacrylamide gel electrophoresis (10% polyacrylamide, 39:1 bis-acrylamide 7 M urea, 1×TBE) and visualized by UV-transillumination after incubation with ethidium bromide. 5 pmole of the RNA template and one aliquot of the FNA-synthesis after alkaline hydrolysis of the template strand (that in case of 100% conversion correspond to 5 pmole) were loaded on the gel. Furthermore approximately 5 pmole of the PCR-reaction and 0.5 μl of the final transcription were loaded onto the gel (FIG. 39).

### Course of Selection

During selection radioactively labeled FNA was used and the binding was calculated as percentage based on the utilized amount of FNA. The radioactivity was determined with a scintillation counter (Beckman, Fullerton, USA). The course of selection is depicted in FIG. 43. After four rounds of selection, a target-specific enrichment (0.89% in the presence of the protein) with a factor of 1.85 compared to the control (0.48% in the absence of protein) could be detected for the first time.

### EXAMPLE 2

### Synthesis of a FNA, DNA, 2′-F-Pyrimidine RNA and RNA Library for Stability Studies

### FNA Library/DNA Library

At first the synthetic DNA library BSA-1A initial library was converted into double-stranded DNA by a Fill-in-reaction using the BSA 1A-Reverse Primer 77. Then the dsDNA was used in the in vitro transcription. BSA-1A FS-Primer 3rG+15 was used as a primer for the FNA synthesis of the FNA library. Subsequent to the FNA synthesis the RNA template strand and the primer were removed by alkaline hydrolysis which led to an FNA library consisting of 63 nucleotides including 40 randomized nucleotides at its 5′-end ((N)40-CACGAGTGAAGTCTGAGCTCC-3′). Finally the sample was precipitated with ethanol for de-salting the sample. The components and reaction conditions correspond to the previous described protocols for FNA-synthesis, alkaline hydrolysis and ethanol precipitation.

For comparison DNA was synthesized in parallel. Instead of the FNA-synthesis a DNA-synthesis according to protocol of the FNA-synthesis was carried out, whereby the FNTPs were substituted for dNTPs (for protocols of all reactions using the herein specified oligonucleotides, see Example 1)

### RNA/2′-F-Pyrimidine RNA Library

As additional controls libraries consisting of RNA and 2′-F-pyrimidine RNA were used. The libraries were synthesized by in vitro transcription. Starting from the BSA-1A initial library a PCR reaction using the BSA-1 FS-Primer 3′G and the BSA1 Reverse Primer T7 was carried out (protocol, see Example 1) and the PCR-product was subjected to an alkaline hydrolysis (in a manner analogous to the description in Example 1 for the alkaline hydrolysis subsequent to the FNA-synthesis) and an in vitro transcription was carried out for the synthesis of the RNA library (protocol, see Example 1) and for the synthesis of a 2′-F-pyrimidine library (protocol, see in the following) (length of the libraries: 63 nucleotides). To avoid any undesired inhibition of the RNases by the RNase-inhibitor “RNaseOut” (Invitrogen), the RNase inhibitor was removed by phenol chloroform extraction and subsequent ethanol precipitation.

Die 2′-F-pyrimidine synthesis was carried out as follows:

| Component | final concentration |
|---|---|
| Txn-buffer with 6 mM MgCl2 (Epicentre, Madison, | 1x |
| USA) | |
| DTT (Merck, Darmstadt, Germany) | 10 mM |
| MgCl2 (Ambion, Austin, USA) | 5 mM |
| ATP (Larova, Teltow, Germany) | 1.5 mM |
| GTP (Larova, Teltow, Germany) | 1.5 mM |
| 2′-F-CTP (TriLink Biotech, San Diego, USA) | 1.5 mM |
| 2′-F-UTP (Noxxon Pharma AG, Berlin, Germany) | 1.5 mM |
| PCR-product BSA-1A Pool | 1 μM |
| T7 R&DNA-polymerase (Epicentre, Madison, USA) | 1.5 U/μl |

Incubation: 4 to 12 hours at 37° C.

### EXAMPLE 3

### DNase I Stability

Each 4 pmole of the FNA library and likewise synthesized DNA library (see Example 2) were incubated for 5 min at 37° C. in 10 μl of a buffer (100 mM NaOAc, 5 mM MgCl₂) including different amounts of DNase I (Sigma) according to the manufacturer's instructions. The dilutions of DNase I were prepared in the same buffer immediately prior to their use.

The DNase digestion was stopped by addition of 6 M urea/15 mM EDTA and by a denaturation step (10 min at 95° C.).

The samples were separated on a 10% denaturing polyacrylamide gel and visualized by a UV-transilluminator upon staining with ethidium bromide (FIG. 40). It was shown that FNA is at least 100 fold more stable against DNase I-digestion than DNA.

### EXAMPLE 4

### RNase Stability

For further characterization of the FNA the RNase stability of the enzymatically synthesized FNA library was determined and compared to RNA and to 2′-F-pyrimidine RNA (synthesis, see Example 2). Each 4 pmole of the FNA library, RNA or 2′-pyrimidine RNA library were incubated according to the manufacturer's instructions for 30 min at 37° C. in 10 μl of a buffer including different amounts of RNase T1 (1-1000 units) and RNase I (0.1-100 units). The RNase T1 originates from Aspergillus oryzae and cleaves after Gs. RNase I originates from E. coli and cleaves RNA in an unspecific manner. Both RNases were recombinantly produced in E. coli by Ambion (Austin, Tex., USA). RNase T1 buffer: 10 mM Tris, pH 8, 100 mM NaCl; RNase I buffer: 50 mM Tris, pH 7.5, 1 mM EDTA.

The reaction was stopped by addition of 20 μl STOP-solution (2% SDS, 50 mM EDTA, 6 M urea) and quick-freeze in a mixture of dry ice and ethanol. In the following the samples were stored on ice until denaturation (5 min at 95° C.), were quickly cooled on ice after denaturation and loaded onto an analytical denaturing 10% polyacrylamide gel.

After separation by gel electrophoresis the bands were stained with ethidium bromide and visualized under UV light on a UV-transilluminator (FIG. 41). In contrast to 2′-F-pyrimidine RNA and RNA (not shown) FNA was not cleaved by the specified RNases under the chosen reaction conditions.

### EXAMPLE 5

### Stability of FNA in Serum

The stability of FNA and RNA (for the synthesis, see Example 2) to serum nucleases was tested in human serum. In order to minimize pH-shifts in the course of the experiment (4 days) 50 mM sodium phosphate buffer was added to the serum. Each 4 pmole of nucleic acid were incubated in 20 μl batches with 14 μl human serum (70% serum) at 37° C. for a length of time as depicted in FIG. 42. After expiration of the reaction time 20 μl STOP-solution (2% SDS, 50 mM EDTA, 6 M urea) were added, the samples were quick-freezed in a bath of ethanol and dry ice and stored at −80° C. until the end of the experiment. In the course of the 96 hours experiment the pH-value of the buffered serum shifted from pH 7.4 to pH 8.5. On the last day of the experiment the samples were extracted with phenol chloroform. All work steps were carried out on ice or in a cooled centrifuge (4° C.). The samples were denatured for 5 min. at 95° C., cooled down on ice and loaded on a 10% denaturing polyacrylamide gel. After separation by gel electrophoresis the bands were stained with ethidium bromide and visualized by UV-light. As depicted in FIG. 42. in contrast to RNA, FNA is not affected by nucleases in the serum under the chosen reaction conditions.

### EXAMPLE 6

### Oligonucleotides as Used

For the different reactions as they were used in the examples the following oligonucleotides were used.

### FNA-Selection 1

### FNA-Selection 1 with FS Primer Consisting of FNA (A)

underlined: T7 RNA polymerase promoter or SP6 RNA polymerase promoter

### N_{OH}: Ribo-nucleotide

BSA-1A FNA-Pool (FNA, 83 nt)
GTGGAACCGACAGTGGTACGTG-N₄₀-CACGAGTGAAGTCTGAGCTCC
BSA-1A initial Library (DNA, 83 nt)
GTGGAACCGACAGTGGTACGTG-N₄₀CACGAGTGAAGTCTGAGCTCC
BSA-1A Forward Primer (DNA, 20 nt)
GTG GAA CCG ACA GTG GTA CG
BSA-1A FS-Primer (FNA, for FNA-synthesis, 20 nt)
GTG GAA CCG ACA GTG GTA CG
BSA1 Reverse Primer T7 (DNA, 38 nt)
TCT AAT ACG ACT CAC TAT AGG AGC TCA GAC TTC ACT CG

used for different tests:

BSA1 FS-Primer 3′rG (DNA, 15 nt)
5′-ACC GAC AGT GGT ACG_{OH}-3′
BSA1 FS Primer 3rG + 15 (DNA, 35 nt)
GTC CTA CCG_{OH} TCA GAT G_{OH}TG GAA CCG_{OH} ACA GTG_{OH}
GTA CG_{OH}

### FNA Selection 1 (B and C) with FS Primer Consisting of RNA or DNA

(using two overlapping primers and a Forward Synthesis Primer consisting of RNA or of DNA with a 3′-terminal ribo-nucleotide)
underlined: T7 RNA polymerase promoter or SP6 RNA polymerase promoter

### N_{OH}: ribo-nucleotide

BSA-1B FNA-Pool (FNA, 74 nt)
ACAGTGGTACGTG-N₄₀-CACGAGTGAAGTCTGAGCTCC
BSA-1B initial Library (DNA, 87 nt)
TGATGTGGAACCGACAGTGGTACGTG-N₄₀-CACGAGTGAAGTCTGAGC
TCC
BSA-1B Forward Primer (DNA, 24 nt)
TGA TGT GGA ACC GAC AGT GGT ACG TG
BSA-1B Reverse Primer T7 (DNA, 38 nt)
TCT AAT ACG ACT CAC TAT AGG AGC TCA GAC TTC ACT CG
BSA-1B FS-Primer (RNA, 13 nt)
UGA UGU GGA ACC G
BSA-1B FS-Primer (DNA with ribo-nucleotides,
13 nt)
TGA TG U_{OH} GGA ACC G_{OH}
BSA-1C FNA-Pool (FNA, 83 nt)
GTGGAACCGACAGTGGTACGTGN40CACGAGTGAAGTCTGAGCTCC
BSA-1C initial Library (DNA, 83 nt)
GTGGAACCGACAGTGGTACGTGN40CACGAGTGAAGTCTGAGCTCC
BSA-1C Forward Primer (DNA, 40 nt)
AAT TGT CCT ACT CGT CAG ATG TGG AAC CGA CAG TGG
TAC G
BSA-1C Reverse Primer T7 (DNA, 38 nt)
TCT AAT ACG ACT CAC TAT AGG AGC TCA GAC TTC ACT CG
BSA-1C FS Primer 3′-rG (DNA with ribo-nucleotides,
21 nt)
AAT TGT CCT ACT CGT CAG ATG_{OH}
BSA-1C FS-Primer 3′-rG (DNA with ribo-nucleotides,
21 nt)
AAT TG_{OH}T CCT ACT CG_{OH}T CAG ATG_{OH}

### FNA Selection 1 Using the STAR-Technology

(using two overlapping primers and a Forward Synthesis Primer consisting of RNA or of DNA including a 3′-terminal ribo-nucleotide)
in italics: Nucleotides of the libraries and of the ligation matrices that hybridize with each other
underlined: T7 RNA-polymerase-promoter or SP6-RNA-polymerase-promoter

### N_{OH}: ribo-nucleotide

pN: 5′-phosphate of a nucleotide
3′dN: 2′-3′-dideoxy-nucleotide

BSA-1 STAR strand during ligation
GCGAGTTCCTCTCAGCGT′ [symbol] -(dN)₄₀- [symbol] ′TATAGTGAGTCGTATTAGTAG TCGC
GGAGAGTCGCA.CCCT CAGG.ATATCACTCAGCATAATCATC AGCG
BSA-1 STAR Library (FNA, 48 nt)
GGGA-(dN)₄₀—GTCC
BSA-1 STAR Forward Primer (DNA, 18 nt)
GCG AGT TCC TCT CAG CGT
BSA-1 STAR Reverse Primer (DNA, including a T7 RNA polymerase
promoter, 29 nt)
GCG ACT ACT AAT ACG ACT CAC TAT AGG AC
BSA-1 STAR Forward Template (DNA, 15 nt)
TCC CAC GCT GAG AG 3′dG
BSA-1 STAR Reverse Ligate (DNA, 25 nt)
pTAT AGT GAG TCG TAT TAG TAG TCG 3′dC
BSA-1 STAR DNA FS Primer (DNA with ribo-nucleotides, 18 nt)
GCG AGT U_{OH} CC TCT CAG CG U_{OH}
BSA-1 STAR RNA FS Primer (RNA, 18 nt)
GCG AGU UCC UCU CAG CGU

### FNA-Selection 2

### FNA Selection 2 with FS Primer Consisting of FNA (A)

(using a Forward Synthesis Primer consisting of FNA)

BSA-2A FNA Pool (FNA, 83 nt)
GTGGAACCGACAGTGGTACGTG-N₄₀-CACGAGTGAAGTCTGAGCTCC
BSA-2A initial Library (DNA, 83 nt)
GTGGAACCGACAGTGGTACGTG-N₄₀-CACGAGTGAAGTCTGAGCTCC
BSA-2A Forward Primer (DNA, 20 nt)
GTG GAA CCG ACA GTG GTA CG
BSA-2A FS-Primer (FNA for FNA-synthesis, 20 nt)
GTG GAA CCG ACA GTG GTA CG
BSA-2A Reverse Primer (DNA, 19 nt)
GG AGC TCA GAC TTC ACT CG

### FNA Selection 2 with FS-Primer Consisting of DNA or RNA (B)

(using one overlapping primer consisting of RNA or of DNA with a 3′-terminal ribo-nucleotide)

### N_{OH}: ribo-nucleotide

BSA-2B FNA-Pool (FNA, 74 nt)
ACAGTGGTACGTG-N₄₀-CACGAGTGAAGTCTGAGCTCC
BSA-2B initial Library (DNA, 87 nt)
TGATGTGGAACCGACAGTGGTACGTG-N₄₀-CACGAGTGAAGTCTGAG
CTCC
BSA-2B Forward Primer (DNA, 24 nt)
TGA TGT GGA ACC GAC AGT GGT ACG TG
BSA-2B Reverse Primer (DNA, 19 nt)
GG AGC TCA GAC TTC ACT CG
BSA-2B RNA FS-Primer (RNA, 13 nt)
UGA UGU GGA ACC G
BSA-2B DNA FS-Primer (DNA with ribo-nucleotides,
13 nt)
TGA TG U_{OH} GGA ACC G_{OH}

### FNA Selection 2 Using the STAR-Technology

(using one overlapping primer consisting of RNA or of DNA with a 3′-terminal ribo-nucleotide)
in italics: Nucleotides of the libraries and of the ligation templates that hybridize with each other

### N_{OH}: Ribo-nucleotides

pN: 5′-Phosphate of a nucleotide
3′dN: 2′-3′-dideoxy-nucleotide

BSA-2 STAR Strand during ligation
GCGAGTTCCTCTCAGCGT′ [symbol] -(dN)₄₀- [symbol] ′TATAGTGAGTCGTATTAGTAGTCGC
GGAGAGTCGCA.CCCT CAGG.ATATCACTCAGCATAATCATCAGCG
BSA-2 STAR Library (FNA, 48 nt)
GGGA-(dN)₄₀-GTCC
BSA-2 STAR Forward Primer (DNA, 18 nt)
GCG AGT TCC TCT CAG CGT
BSA-2 STAR Reverse Primer (DNA with ribo-nucleotides, 29 nt)
GCG ACT ACT AAU_{OH} ACG ACT CAC TAT I_{OH} GG AC
BSA-2 STAR Forward Template (DNA, 15 nt)
TCC CAC GCT GAG AG 3′dG
BSA-2 STAR Reverse Ligate (DNA, 25 nt)
pTAT AGT GAG TCG TAT TAG TAG TCG 3′dC
BSA-2 STAR RNA FS Primer (RNA, 18 nt)
GCG AGU UCC UCU CAG CGU
BSA-2 STAR DNA FS Primer (DNA with ribo-nucleotides, 18 nt)
GCG AGT U_{OH}CC TCT CAG CGU_{OH}
FNA Selektion 3
BSA-3 FNA Pool (FNA, 83 nt)
5′-GTGGAACCGACAGTGGTACGTG-N₄₀-CACGAGTGAAGTCTGAGCTCC-3′
BSA-3 initial Library (DNA, 83 nt)
5′-GTGGAACCGACAGTGGTACGTG-N₄₀-CACGAGTGAAGTCTGAGCTCC-3′
BSA-3 Forward Primer (FNA, 20 nt)
5′-GTG GAA CCG ACA GTG GTA CG-3′
BSA-3 Reverse Primer (DNA, 19 nt)
5′Biotin-GG AGC TCA GAC TTC ACT CG-3′
BSA-3 Forward Sequencing Primer (DNA, 20 nt)
5′-GTG GAA CCG ACA GTG GTA CG-3′
BSA-3 Reverse Sequencing Primer (DNA, 19 nt)
5′-GG AGC TCA GAC TTC ACT CG-3′

### DNA Selection with FS Primer Consisting of DNA

underlined: T7 RNA polymerase promoter or SP6 RNA polymerase promoter

DNA-Pool (DNA, 83 nt)
GTGGAACCGACAGTGGTACGTG-N₄₀-CACGAGTGAAGTCTGAGCTCC
DNA initial Library (DNA, 83 nt)
GTGGAACCGACAGTGGTACGTG-N₄₀-CACGAGTGAAGTCTGAGCTCC
DNA Forward Primer (DNA, 20 nt)
GTG GAA CCG ACA GTG GTA CG
DNA Reverse Primer T7 (DNA, 38 nt)
TCT AAT ACG ACT CAC TAT AGG AGC TCA GAC TTC ACT CG

### LITERATURE

The various references which are contained herein read completely as follows and their disclosure is incorporated herein by reference.
Aurup, H., Williams, D. M. and Eckstein, F. (1992) 2′-Fluoro- and 2′-amino-2′-deoxynucleoside 5′-triphosphates as substrates for T7 RNA polymerase. Biochemistry, 31, 9636-9641.
Bell, C., Lynam, E., Landfair, D. J., Janjic, N. and Wiles, M. E. (1999) Oligonucleotide NX1838 inhibits VEGF165-mediated cellular responses in vitro. In Vitro Cell Dev Biol Anim, 35, 533-542.
Bock, L. C., Griffin, L. C., Latham, J. A., Vermaas, E. H. and Toole, J. J. (1992) Selection of single-stranded DNA molecules that bind and inhibit human thrombin. Nature, 355, 564-566.
Chiu, Y. L. and Rana, T. M. (2003) siRNA function in RNAi: A chemical modification analysis. Rna, 9, 1034-1048.
Cummins, L. L., Owens, S. R., Risen, L. M., Lesnik, E. A., Freier, S. M., McGee, D., Guinosso, C. J. and Cook, P. D. (1995) Characterization of fully 2′-modified oligoribonucleotide hetero- and homoduplex hybridization and nuclease sensitivity. Nucleic Acids Res, 23, 2019-2024.
Eaton, B. E. and Pieken, W. A. (1995) Ribonucleosides and RNA. Annu Rev Biochem, 64, 837-863.
Green, L. S., Jellinek, D., Bell, C., Beebe, L. A., Feistner, B. D., Gill, S. C., Jucker, F. M. and Janjic, N. (1995) Nuclease-resistant nucleic acid ligands to vascular permeability factor/vascular endothelial factor. Chem Biol, 2, 683-695.
Green, L. S., Jellinek, D., Jenison, R., Ostman, A., Heldin, C. H. and Janjic, N. (1996) Inhibitory DNA ligands to platelet-derived growth factor B-chain. Biochemistry, 35, 14413-14424.
Griffin, L. C., Tidmarsh, G. F., Bock, L. C., Toole, J. J. and Leung, L. L. (1993) In vivo anticoagulant properties of a novel nucleotide-based thrombin inhibitor and demonstration of regional anticoagulation in extracorporeal circuits. Blood, 81, 3271-3276.
Henry, S. P., Geary, R. S., Yu, R. and Levin, A. A. (2001) Drug properties of second-generation antisense oligonucleotides: how do they measure up to their predecessors? Curr Opin Investig Drugs, 2, 1444-1449.
Jellinek, D., Green, L. S., Bell, C., Lynott, C. K., Gill, N., Vargeese, C., Kirschenheuter, G., McGee, D. P. C., Abesinghe, P., Pieken, W. A., Shapiro, R., Rifkin, D. B., Moscatelli, D. and Janjic, N. (1995) Potent 2′-amino-2′deoxypyrimidine RNA inhibitors of basic fibroblast growth factor. Biochemistry, 34, 11363-11372.
Jhaveri, S., Olwin, B. and Ellington, A. D. (1998) In vitro selection of phosphorothiolated aptamers. Bioorg Med Chem Lett, 8, 2285-2290.
King, D. J., Bassett, S. E., Li, X., Fennewald, S. A., Herzog, N. K., Luxon, B. A., Shope, R. and Gorenstein, D. G. (2002) Combinatorial selection and binding of phosphorothioate aptamers targeting human NF-kappa B RelA(p65) and p50. Biochemistry, 41, 9696-9706.
Kujau, M. J., Siebert, A. and Wolfi, S. (1997) Design of leader sequences that improve the efficiency of the enzymatic synthesis of 2′-amino-pyrimidine RNA for in vitro selection. J Biochem Biophys Methods, 35, 141-151.
Kusser, W. (2000) Chemically modified nucleic acid aptamers for in vitro selections: evolving evolution. J Biotechnol, 74, 27-38.
Leva, S., Lichte, A., Burmeister, J., Muhn, P., Jahnke, B., Fesser, D., Erfurth, J., Burgstaller, P. and Klussmann, S. (2002) GnRH binding RNA and DNA Spiegelmers: a novel approach toward GnRH antagonism. Chem Biol, 9, 351-359.
Lin, Y., Qiu, Q., Gill, S. C. and Jayasena, S. D. (1994) Modified RNA sequence pools for in vitro selection. Nucleic Acids Research, 22, 5229-5234.
Manoharan, M. (1999) 2′-carbohydrate modifications in antisense oligonucleotide therapy: importance of conformation, configuration and conjugation. Biochim Biophys Acta, 1489, 117-130.
Meador, J. W., 3rd, McElroy, H. E., Pasloske, B. L., Milburn, S. C. and Winkler, M. M. (1995) pTRIPLEscript: a novel cloning vector for generating in vitro transcripts from tandem promoters for SP6, T7 and T3 RNA polymerase. Biotechniques, 18, 152-157.
Milligan, J. F. and Uhlenbeck, O. C. (1989) Synthesis of small RNAs using T7 RNA polymerase. Methods Enzymol, 180, 51-62.
Murphy, M. B., Fuller, S. T., Richardson, P. M. and Doyle, S. A. (2003) An improved method for the in vitro evolution of aptamers and applications in protein detection and purification. Nucleic Acids Res, 31, el 10.
Ono, T., Scalf, M. and Smith, L. M. (1997) 2′-Fluoro modified nucleic acids: polymerase-directed synthesis, properties and stability to analysis by matrix-assisted laser desorption/ionization mass spectrometry. Nucleic Acids Res, 25, 4581-4588.
Padilla, R. and Sousa, R. (1999) Efficient synthesis of nucleic acids heavily modified with non-canonical ribose 2′-groups using a mutantT7 RNA polymerase (RNAP). Nucleic Acids Res, 27, 1561-1563.
Padilla, R. and Sousa, R. (2002) A Y639F/H784A T7 RNA polymerase double mutant displays superior properties for synthesizing RNAs with non-canonical NTPs. Nucleic Acids Res, 30, e138.
Richardson, F. C., Kuchta, R. D., Mazurkiewicz, A. and Richardson, K. A. (2000) Polymerization of 2′-fluoro- and 2′-O-methyl-dNTPs by human DNA polymerase alpha, polymerase gamma, and primase. Biochem Pharmacol, 59, 1045-1052.
Stryer, L. (1995) Biochemistry. W. H. Freeman and Company, New York.
Williams, K. P., Liu, X. H., Schumacher, T. N., Lin, H. Y., Ausiello, D. A., Kim, P. S, and Bartel, D. P. (1997) Bioactive and nuclease-resistant L-DNA ligand of vasopressin. Proc Natl Acad Sci USA, 94, 11285-11290.
Xu, Y., Zhang, H. Y., Thormeyer, D., Larsson, O., Du, Q., Elmen, J., Wahlestedt, C. and Liang, Z. (2003) Effective small interfering RNAs and phosphorothioate antisense DNAs have different preferences for target sites in the luciferase mRNAs. Biochem Biophys Res Commun, 306, 712-717.
Zhang, H. Y., Mao, J., Zhou, D., Xu, Y., Thonberg, H., Liang, Z. and Wahlestedt, C. (2003) mRNA accessible site tagging (MAST): a novel high throughput method for selecting effective antisense oligonucleotides. Nucleic Acids Res, 31, e72.

The features of the invention disclosed in the preceding description, the claims and the figures can be essential for the practice of the invention either alone or in any combination.

1. A method for the synthesis of a nucleic acid, whereby the nucleic acid comprises modified nucleotides, comprising the steps of:
providing a template strand;
providing a primer which is at least partially hybridizing to the template strand;
providing nucleoside triphosphates, whereby a portion of the nucleoside triphosphates are modified nucleoside triphosphates;
providing a polymerase activity; and
incubating the template strand, the primers, the nucleoside triphosphates for the synthesis of a nucleic acid which is essentially complementary to the template strand,
characterized in that the polymerase activity is a reverse transcriptase activity.

2. The method according to claim 1, characterized in that the reverse transcriptase is selected from the group comprising reverse transcriptases of murine moloney leukemia virus (MMLV), avian myeloblastosis virus (AMV), thermostable reverse transcriptases, DNA polymerase of Carboxydothermus hydrogenoformans, respective mutants thereof, and mixtures thereof.

3. The method according to claim 1 or 2, characterized in that the modified nucleoside triphosphates are selected from the group comprising 2′-fluoro-modified nucleoside triphosphates, 2′-amino-modified nucleoside triphosphates, 2′-azido-modified nucleoside triphosphates, 2′-O-methyl-modified nucleoside triphosphates, 2′-alkyl-modified nucleoside triphosphates, 2′-allyl-modified nucleoside triphosphates, arabino-nucleoside triphosphates and nucleotide phosphorothioates.

4. The method according to any of claims 1 to 3, characterized in that the modified nucleoside triphosphates are 2′-fluoro nucleoside triphosphates.

5. The method according to any of claims 1 to 4, characterized in that the nucleoside triphosphates provided are exclusively modified nucleoside triphosphates and that the synthesized nucleic acid preferably essentially consists solely of modified nucleotides.

6. The method according to any of claims 1 to 5, characterized in that the template strand consists of RNA.

7. The method according to any of claims 1 to 5, characterized in that the template strand consists of DNA.

8. The method according to any of claims 1 to 5, characterized in that the template strand consists of a modified nucleic acid, preferably a 2′-fluoro nucleic acid.

9. The method according to any of claims 1 to 8, characterized in that the sequence of the primer is part of the nucleic acid to be synthesized.

10. The method according to any of claims 1 to 8, characterized in that the sequence of the primer is different from the nucleic acid to be synthesized.

11. The method according to any of claims 1 to 9, characterized in that the primer consists of modified nucleoside phosphates, whereby the modification of the nucleoside phosphates of the primer is the same modification as the one of the nucleoside triphosphates provided.

12. The method according to any of claims 1 to 8 and 10, characterized in that the primer consists of RNA.

13. The method according to any of claims 1 to 8 and 10, characterized in that the primer consists of DNA, whereby at least the 3′ terminal nucleotide of the primer is a deoxyribonucleotide.

14. The method according to any of claims 1 to 13, characterized in that the polymerase activity synthesizes a strand which is essentially complementary to the template strand, whereby it is preferably base paired with the template strand.

15. The method according to claim 14, characterized in that the synthesized nucleic acid is separated from the template strand.

16. The method according to any of claims 1 to 15, characterized in that the primer or a part thereof is removed from the nucleic acid which is synthesized by the polymerase activity.

17. The method according to any of claims 1 to 16, characterized in that the template strand and/or the primer is digested or cleaved, preferably after the synthesis of the nucleic acid which is essentially complementary to the template strand.

18. The method according to any of claims 15 to 17, characterized in that the separation and/or the cleavage is performed by alkaline cleavage or enzymatic activity.

19. Use of a reveres transcriptase for the synthesis of a nucleic acid, whereby the nucleic acid comprises at least a modified nucleoside phosphate.

20. Use according to claim 19, characterized in that the reverse transcriptase is selected from the group comprising reverse transcriptases of murine moloney leukemia virus (MMLV), avian myeloblastosis virus (AMV), thermostable reverse transcriptases, DNA polymerase of Carboxydothermus hydrogenoformans, mutants thereof, and mixtures thereof.

21. Use according to claim 19 or 20, characterized in that the modified nucleoside triphosphate is selected from the group comprising 2′-fluoro-modified nucleoside triphosphates, 2′-amino-modified nucleoside triphosphates, 2′-azido-modified nucleoside triphosphates, 2′-O-methyl-modified nucleoside triphosphates, 2′-alkyl-modified nucleoside triphosphates, 2′-allyl-modified nucleoside triphosphates, arabino nucleoside triphosphates and nucleoside phosphorothioates.

22. Use according to any of claims 19 to 21, characterized in that the nucleic acid essentially consists completely of modified nucleoside phosphates.

23. A method for the selection of a target molecule binding nucleic acid, in particular of aptamers, comprising the steps of:
(a) providing a heterogeneous population of nucleic acids, in particular D-nucleic acids, whereby any of the nucleic acids comprises a region having a randomized sequence and a first constant sequence at the 5′ end and a second constant sequence at the 3′ end, and whereby the nucleic acids forming the population differ in the randomized sequence,
(b) contacting the population of nucleic acids with the target molecule,
(c) separating the nucleic acids not interacting with the target molecule,
(d) separating from the target molecule the nucleic acid(s) interacting with the target molecule,
(e) optionally repeating the steps (a) to (d), whereby the nucleic acid(s) from step
(d) form the heterogeneous population or is/are contained therein,
(f) reverse transcription of the nucleic acid(s) which was/were interacting with the target molecule, in order to form reverse transcription products,
(g) performing a second strand synthesis, whereby the second strand is essentially complementary to the reverse transcription products, whereby the second strand synthesis is preferably an amplification reaction and preferably a polymerase chain reaction,
(h) transcription of the product of (g), whereby the synthesized second strand serves as a template strand, to obtain transcription products,
(i) synthesis of the nucleic acids which are essentially complementary to the transcription products,
(j) optionally repeating steps (a) to (i), whereby the nucleic acid(s) of step (i) form the heterogeneous population or are contained therein, and
(k) optionally sequencing the nucleic acid(s) obtained from step (f) or (g),
characterized in that the synthesis according to step (i) is performed in accordance with a method according to any of claims 1 to 22.

24. A method for the selection of a target molecule binding nucleic acid, in particular of aptamers, comprising the steps of:
(a) providing a heterogeneous population of nucleic acids, in particular D-nucleic acids, whereby any of the nucleic acids comprises a region having a randomized sequence and a first constant sequence at the 5′ end and a second constant sequence at the 3′ end, and whereby the nucleic acids forming the population differ in the randomized sequence,
(b) contacting the population of nucleic acids with the target molecule,
(c) separating the nucleic acids not interacting with the target molecule,
(d) separating from the target molecule the nucleic acid(s) interacting with the target molecule,
(e) optionally repeating steps (a) to (d), whereby the nucleic acid(s) of step (d) form the heterogeneous population or are contained therein,
(f) reverse transcription of the nucleic acid(s) which was/were interacting with the target molecule, in order to form reverse transcription products,
(g) performing a second strand synthesis, whereby the second strand is essentially complementary to the reverse transcription products, whereby the second strand synthesis is preferably an amplification reaction and preferably a polymerase chain reaction,
(h) transcription of the product of step (g), whereby the synthesized second strand serves as a template strand in order to obtain transcription products,
(i) synthesis of nucleic acids which are essentially complementary to the transcription products,
(j) optionally repeating steps (a) to (i), whereby the nucleic acid(s) of step (i) form the hydrogenous population or are contained therein, and
(k) optionally sequencing the nucleic acid(s) obtained from step (f) or (g),
characterized in that at least the randomized region of the nucleic acid and/or of the nucleic acid synthesized in step (i) essentially consists completely of modified nucleoside phosphates, preferably 2′-fluoro nucleoside phosphates.

25. A method for the selection of a target molecule binding nucleic acid, in particular of aptamers, in particular a method according to any of claims 23 or 24, comprising the steps of:
(a) providing a heterogeneous population of nucleic acids, in particular D-nucleic acids, whereby any of the nucleic acids comprises a region having a randomized sequence and a first constant sequence at the 5′ end and a second constant sequence at the 3′ end and whereby the nucleic acids forming the population differ in the randomized sequence,
(b) contacting the population of nucleic acids with the target molecule,
(c) separating the nucleic acids not interacting with the target molecule,
(d) separating from the target molecule the nucleic acid(s) interacting with the target molecule,
(e) optionally repeating steps (a) to (d), whereby the nucleic acid(s) of step (d) form the heterogeneous population or are contained therein,
(f) reverse transcription of the nucleic acid(s) which was/were interacting with the target molecule, in order to form reverse transcription products,
(g) performing a second strand synthesis, whereby the second strand is essentially complementary to the reverse transcription products, whereby the second strand synthesis is preferably an amplification reaction and preferably a polymerase chain reaction,
(h) transcription of the product of step (g), whereby the synthesized second strand serves as a template strand in order to obtain transcription products,
(i) synthesis of nucleic acids which are essentially complementary to the transcription products,
(j) optionally repeating steps (a) to (i), whereby the nucleic acid(s) of step (i) form the hydrogenous population or are contained therein, and
(k) optionally sequencing the nucleic acid(s) obtained from step (f) or (g),
characterized in that
the first constant sequence of the nucleic acid in step (a) comprises a forward primer sequence and the second constant sequence comprises a reverse primer binding site, and
a reverse primer is used in the reverse transcription according to step (f) which is essentially complementary to the reverse primer binding site and comprises at its 5′ end a further partial region, and the reverse transcription product comprises in 5′→3′ direction a reverse primer sequence, a sequence essentially complementary to the randomized sequence and a forward primer binding site.

26. The method according to claim 25, characterized in that the reverse primer and a forward primer are used in the second strand synthesis, whereby the forward primer is at least partially complementary to a part of the forward primer binding site of the reverse transcription product, whereby the sequence of the synthesized second strand is essentially identical to the sequence of the nucleic acid of step (d) and additionally comprises at the 3′ end a sequence which is essentially complementary to the further partial region of the reverse primer.

27. The method according to claims 25 or 26, characterized in that the further partial region of the reverse primer is a promoter sequence, whereby preferably the promoter sequence is selected from the group comprising promoter sequences of the T7-RNA polymerase, the T3-RNA polymerase and the SP6 polymerase.

28. The method according to any of claims 25 to 27, characterized in that the strand synthesized in the second strand synthesis is used as a template strand in a transcription reaction, whereby the transcription product comprises in 3′→5′ direction the forward primer binding site, the complementary randomized sequence and the reverse primer sequence.

29. The method according to claim 28, characterized in that the transcription product is reacted with a reverse transcriptase together with a forward synthesis primer and modified nucleoside triphosphates, preferably 2′-fluoro nucleoside phosphates, whereby the forward synthesis primer hybridizes to the forward primer binding site in order to obtain a synthesis product, whereby the synthesis product comprises modified nucleoside phosphates, preferably 2′-fluoro nucleoside phosphates.

30. The method according to claim 29, characterized in that the forward synthesis primer consists of modified nucleoside triphosphates.

31. The method according to any of claims 25 to 30, characterized in that the template strand is subjected to an alkaline treatment in order to obtain a single-stranded nucleic acid, whereby the nucleic acid comprises in 5′→3′ direction the forward primer sequence, the randomized region and the reverse primer binding site.

32. The method according to any of claims 25 to 27, characterized in that the forward primer comprises at its 5′ end a further partial region and that the synthesized second strand comprises at its 5′ end a sequence corresponding to the further partial region.

33. The method according to claim 32, characterized in that the strand synthesized in the second strand synthesis is subjected to a transcription reaction as a template strand, whereby the transcription product comprises in 3′→5′ direction the forward primer binding site including the sequence complementary to the further partial region of the forward primer, the complementary randomized region and the reverse primer sequence at its 5′ end, whereby the reverse primer sequence preferably lacks a sequence corresponding to the further partial region of the reverse primer.

34. The method according to claim 33, characterized in that the transcription product is reacted with a reverse transcriptase together with a forward synthesis primer and modified nucleoside triphosphates, preferably 2′-fluoro nucleoside phosphates, whereby the forward synthesis primer is hybridized to the forward primer binding site, in order to obtain a synthesis product, whereby the synthesis product comprises modified nucleoside phosphates, preferably 2′-fluoro nucleoside phosphates.

35. The method according to claim 34, characterized in that the forward synthesis primer consists of ribonucleotides or of deoxyribonucleotides having at least one ribonucleotide at its 3′ end.

36. The method according to any of claims 32 to 35, characterized in that the template strand is subjected to an alkaline cleavage and the forward synthesis primer is cleaved off, in order to obtain a single-stranded nucleic acid, whereby the nucleic acid comprises in 5′→3′ direction the forward primer sequence, the randomized region and the reverse primer binding site.

37. The method according to any of claims 25 to 36, characterized in that the forward primer and the reverse primer consist of DNA.

38. A method for the selection of a target molecule binding nucleic acid, in particular of aptamers, comprising the steps of:
(a) providing a heterogeneous population of nucleic acids, in particular D-nucleic acids, whereby any of the nucleic acids comprises a region having a randomized sequence and a first constant sequence at the 5′ end and a second constant sequence at the 3′ end, and whereby the nucleic acids forming the population differ in the randomized sequence,
(b) contacting the population of nucleic acids with the target molecule,
(c) separating the nucleic acids not interacting with the target molecule,
(d) separating from the target molecule the nucleic acid(s) interacting with the target molecule,
(e) optionally repeating steps (a) to (d), whereby the nucleic acid(s) of step (d) form the heterogeneous population or are contained therein,
(f) reverse transcription of the nucleic acid(s) which was/were interacting with the target molecule, in order to form reverse transcription products,
(g) performing an amplification reaction with the reverse transcription products, whereby the amplification reaction is preferably a polymerase chain reaction, in order to obtain an amplified reverse transcription product,
(h) synthesis of nucleic acids which are essentially complementary to the reverse transcription products amplified in (g), in order to obtain a synthesis product,
(i) optionally repeating steps (a) to (h), whereby the nucleic acid(s) of step (h) form the heterogeneous population or is contained therein,
(j) optionally sequencing of the nucleic acid obtained in step (f) or (g),
characterized in that
the first constant sequence of the nucleic acid in step (a) comprises a forward primer sequence and the second constant sequence comprises a reverse primer binding site, and
a reverse primer is used in the reverse transcription of step (f) which is essentially complementary to the reverse primer binding site and whereby the reverse transcription product comprises in 5′→3′ direction a reverse primer sequence, a sequence essentially complementary to the randomized sequence and a forward primer binding site essentially complementary to the forward primer sequence.

39. The method according to claim 38, characterized in that the reverse primer and a forward primer are used in the second strand synthesis, whereby the forward primer is essentially complementary to the forward primer binding site, whereby the sequence of the synthesized second strand is essentially identical to the nucleic acid to be amplified.

40. The method according to claim 38 or 39, characterized in that in the synthesis after step (g) the amplified reverse transcription product is reacted with a forward synthesis primer, modified nucleoside triphosphates, preferably 2′-fluoro nucleoside triphosphates, and a reverse transcriptase, whereby the forward synthesis primer hybridizes to the forward primer binding site, in order to obtain a synthesis product, whereby the synthesis product comprises modified nucleoside phosphates, preferably 2′-fluoro nucleoside phosphates.

41. The method according to any of claims 38 to 40, characterized in that the forward synthesis primer consists of modified nucleoside triphosphates.

42. The method according to any of claims 38 to 41, characterized in that the template strand is subjected to digestion, preferably an enzymatic digestion, in order to obtain a single-stranded nucleic acid, whereby the nucleic acid comprises in 5′→3′ direction the forward primer sequence, the randomized region and the reverse primer binding site.

43. The method according to claim 38, characterized in that, in the second strand synthesis, the reverser primer and a forward primer are used, whereby the forward primer is essentially complementary to the forward primer binding site and comprises at its 5′ end a further partial region, whereby the partial region preferably has a length of about 10 to 25 and more preferably a length of about 10 to 15 nucleotides, whereby the partial region preferably is a binding site or a part thereof, for a forward synthesis primer, and an extended reverse transcription product is obtained, whereby the extended reverse transcription product corresponds to the reverse transcription product, whereby the reverse transcription product is supplemented at its 3′ end by a sequence, whereby the sequence is complementary to the sequence of the further partial region of the forward primer.

44. The method according to claim 43, characterized in that in the synthesis after step (g) the amplified reverse transcription product is reacted with a forward synthesis primer, modified nucleoside triphosphates, preferably 2′-fluoro nucleoside triphosphates, and a reverse transcriptase, whereby the forward synthesis primer hybridizes to the binding site for the forward synthesis primer in order to obtain a synthesis product, whereby the synthesis product comprises modified nucleoside phosphates, preferably 2′-fluoro nucleoside phosphates.

45. The method according to claim 44, characterized in that the forward synthesis primer consists of ribonucleotides or of deoxyribonucleotides having at least one ribonucleotide at its 3′ end.

46. The method according to any of claims 43 to 45, characterized in that the template strand is subjected to a digestion, preferably an enzymatic digestion, in order to obtain a single-stranded nucleic acid, whereby the nucleic acid comprises in 5′→3′ direction the forward primer sequence, the randomized region and the reverse primer binding site.

47. The method according to any of claims 38 to 46, characterized in that the forward primer and the reverse primer consist of DNA.

48. A method for the selection of a target molecule binding nucleic acid, particularly of aptamers, comprising the steps of:
(a) providing a heterogeneous population of nucleic acids, in particular D-nucleic acids, whereby any of the nucleic acids comprises a region having a randomized sequence and a first constant sequence at the 5′ end and a second constant sequence at the 3′ end and whereby the nucleic acids forming the population differ in the randomized sequence,
(b) contacting the population of nucleic acids with the target molecule,
(c) separating the nucleic acids not interacting with the target molecule,
(d) separating from the nucleic acid the nucleic acid(s) interacting with the nucleic acid,
(e) optionally repeating steps (a) to (d), whereby the nucleic acid(s) of step (d) form the heterogeneous population or are contained therein,
(f) amplifying the nucleic acid of step (a) comprising the step of:
reacting the nucleic acid of step (e) with a reverse transcriptase, a reverse primer, a forward primer and nucleoside phosphates, preferably modified nucleoside phosphates and more preferably 2′-F-nucleoside phosphates,
whereby the reverse primer is essentially complementary to the reverse primer binding site and hybridizes thereto and carries a label, whereby the label is mediating an interaction between the primer and the interaction partner, and
whereby the forward primer is essentially identical to the forward primer sequence of the nucleic acid of step (a),
in order to obtain a double-stranded amplification product, whereby one strand essentially corresponds to the nucleic acid of step (a) and a strand is complementary thereto, whereby the complementary strand carries the label,
(g) removing the complementary strand from the amplification product in order to obtain a nucleic acid corresponding essentially to the nucleic acid of step (a),
(h) optionally repeating steps (a) to (g), whereby the nucleic acid of step (g) forms the heterogeneous population or is contained therein,
(i) optionally sequencing the nucleic acid(s) obtained from step (d), (f) or (g), whereby in case of sequencing preferably the following additional steps are performed:
(ia) reverse transcription using the reverse primer, whereby the reverse primer consists of DNA and does not carry any label,
(ib) amplifying the reverse transcription product of step (ia) by performing a second strand synthesis for the amplification, whereby the reverse primer and the forward primer are used, and whereby the reverse primer does not have any label and the forward primer consists of DNA.

49. The method according to claim 48, characterized in that the complementary strand in step (g) is separated by interaction between the label and the interaction partner.

50. The method according to claim 49, characterized in that the interaction partner is immobilized to a surface.

51. The method according to claim 50, characterized in that the amplification product is immobilized at the surface by the interaction between the label and the interaction partner.

52. The method according to any of claims 49 to 51, characterized in that the two strands of the amplification product are separated from each other, whereby preferably the complementary strand remains immobilized.

53. The method according to any of claims 48 to 52, characterized in that the label is selected from the group comprising biotin, digoxigenin and linker having reactive functional groups and whereby the reactive functional groups are preferably selected from the group comprising amino, carboxy, epoxy and thiol.

54. The method according to any of claims 48 to 53, characterized in that the interaction partner is selected from the group comprising streptavidin, avidin, neutravidin und anti-digoxigenin antibodies and complementary functional groups, and whereby the reactive functional groups are preferably selected from the group comprising amino, carboxy, epoxy and thiol.

55. The method according to any of claims 48 to 53, characterized in that the label is attached at the 5′ and of the reverse primer.

56. The method according to any of claims 48 to 55, characterized in that the forward primer comprises modified nucleoside phosphates, in particular 3′-fluoro nucleoside phosphates.

57. The method according to any of claims 48 to 56, characterized in that the reverse primer comprises deoxynucleoside phosphates.

58. A method for the selection of a target molecule binding nucleic acid, in particular of aptamers, comprising
(a) providing a heterogeneous population of nucleic acids, in particular D-nucleic acids, whereby any of the nucleic acids comprises a region having a randomized sequence and a first constant sequence at the 5′ end and a second constant sequence at the 3′ end and whereby the nucleic acids forming the population differ in the randomized sequence, whereby the nucleic acid comprises modified nucleoside phosphates, preferably 2′-fluoro-modified nucleoside phosphates, and each of the constant sequences comprises 4 to 6 nucleotides,
(b) contacting the population of nucleic acids with the target molecule,
(c) separating the nucleic acids not interacting with the target molecule,
(d) separating from the target molecule the nucleic acid(s) interacting with the target molecule,
(e) optionally repeating steps (a) to (d), whereby the nucleic acid(s) of step (d) form the heterogeneous population or are contained therein,
(f) modifying the nucleic acid of step (a) or (d) by the following steps:
(f0) 5′ phosphorylating the 5′ terminal nucleotide of the nucleic acid of step (a), preferably by using a kinase, under the proviso that the 5′ terminal nucleotide does not already have a phosphate group at the 5′ end,
(fa) providing a first adapter molecule, whereby the first adapter molecule consists of a double-stranded nucleic acid of a first and a second nucleic acid strand and whereby the first nucleic acid strand and the second nucleic acid strand are independently a deoxyribonucleic acid, a ribonucleic acid or an FNA, and whereby the 5′ end of the second nucleic acid strand provides for an overhang, whereby the overhang is at least partially complementary to the first constant partial region of the nucleic acid of step (a) and/or (d) or a part thereof,
(fb) providing a second adapter molecule, whereby the second adapter molecule consists of a double-stranded nucleic acid of a first and a second nucleic acid strand, whereby the first nucleic acid strand carries a 5′ phosphate and the first and the second nucleic acid strand are independent from each other a deoxyribonucleic acid, a ribonucleic acid or an FNA, and whereby the 3′ end of the second nucleic acid strand provides for an overhang which is at least partially complementary to the second constant partial sequence of the nucleic acid of step (a) and/or (d) or a part thereof,
(fc) ligating the first nucleic acid strand of the first and of the second adapter molecule to the nucleic acid of step (a) and/or (d), in order to obtain a ligation product as a reaction product,
(g) reverse transcription of the ligation product by using the second strand of the second adapter molecule present in the ligation reaction as a primer, in order to obtain a reverse transcription product,
(h) performing a second strand synthesis, whereby the second strand is essentially complementary to the reverse transcription product, whereby the second strand synthesis is more preferably an amplification reaction and preferably a polymerase chain reaction,
(i) transcription of the product of (h), whereby the synthesized second strand serves as a template strand in order to obtain transcription products, whereby a transcription product is obtained which is complementary to
the sequence of the first nucleic acid strand of the first adapter molecule,
the first constant partial sequence,
the randomized region, and
the second constant partial sequence; and
(j) performing a nucleic acid synthesis, whereby the transcription product of step (i) is reacted with a forward synthesis primer, modified nucleoside triphosphates, preferably 2′-fluoro nucleoside triphosphates, and a reverse transcriptase, whereby the primer hybridizes to the complementary sequence of the first nucleic acid strand of the first adapter molecule, and whereby the primer consists of RNA or a combination of RNA and DNA, under the proviso that in case of a combination of RNA and DNA at least the 3′ end is formed by a ribonucleotide,
(k) cleaving off the transcription product after step (j) and the forward primer sequence of the nucleic acid molecule synthesized in step (j), in order to obtain a nucleic acid which is essentially identical to the nucleic acid of step (a) or (d),
(l) optionally repeating steps (a) to (k), whereby the nucleic acid of step (k) forms the heterogeneous population or is contained therein, and
(m) optionally sequencing the nucleic acid obtained in step (h).

59. The method according to claim 58, characterized in that the cleavage in step (k) is an alkaline cleavage and/or is performed by RNAase digestion.

60. A method for the selection of a target molecule binding nucleic acid, in particular of aptamers, comprising
(a) providing a heterogeneous population of nucleic acids, in particular D-nucleic acids, whereby any of the nucleic acids comprises a region having a randomized sequence and a first constant sequence at the 5′ end and a second constant sequence at the 3′ end and whereby the nucleic acids forming the population differ in the randomized sequence, whereby the nucleic acid comprises modified nucleoside phosphates, preferably 2′-fluoro-modified nucleoside phosphates, and the constant sequences each comprises 4 to 6 nucleotides and the nucleic acid bears an OH group at the 3′ end,
(b) contacting the population of nucleic acids with the target molecule,
(c) separating the nucleic acids not interacting with the target molecule,
(d) separating from the target molecule the nucleic acid(s) interacting with the target molecule,
(e) optionally repeating steps (a) to (d), whereby the nucleic acid(s) of step (d) form the heterogeneous population or are contained therein,
(f) modifying the nucleic acid of step (a) or (d) by the following steps:
(fa) phosphorylating the 5′ end of the nucleic acid under the proviso that the nucleic acid does not have a phosphate at the 5′ end,
(fb) providing a first adapter molecule, whereby the first adapter molecule consists of a double-stranded nucleic acid of a first and a second nucleic acid strand, and whereby the first nucleic acid strand and the second nucleic acid strand are independent from each other a deoxyribonucleic acid, a ribonucleic acid or an FNA and whereby the 5′ end of the second nucleic acid strand provides for an overhang, whereby the overhang is at least partially complementary to the first constant partial sequence of the nucleic acid of step (a) and/or (d) or a part thereof,
(fc) providing a second adapter molecule, whereby the second adapter molecule consists of a double-stranded nucleic acid of a first and a second nucleic acid strand, whereby the first nucleic acid strand carries a 5′ phosphate and the first and the second nucleic acid strand are independent from each other a deoxyribonucleic acid, a ribonucleic acid or an FNA, and whereby the 3′ end of the second nucleic acid strand provides for an overhang which is at least partially complementary to the second constant partial sequence of the nucleic acid of step (a) and/or (d) or a part thereof and whereby the second nucleic acid strand contains a cleavage site which, upon cleavage of the nucleic acid strand, provides for a first cleavage product and a second cleavage product, whereby the first cleavage product is the 3′ end of the second nucleic acid strand of the second adapter molecule which is at least partially complementary to the second constant partial sequence of the nucleic acid of step (a) and/or (d),
(fd) ligating the first nucleic acid strand of the first and of the second adapter molecule to the nucleic acid of step (a) and/or (d), in order to obtain a ligation product as a reaction product,
(g) reverse transcription of the ligation product by using the second strand of the second adapter molecule present in the ligation reaction as a primer, in order to obtain a reverse transcription product,
(h) performing a second strand synthesis, whereby the second strand is essentially complementary to the reverse transcription product, whereby the second strand synthesis is preferably an amplification reaction and more preferably a polymerase chain reaction, and provides for an amplified reverse transcription product,
(i) degradation of the reverse transcription product, in particular of the amplified reverse transcription product, whereby a nucleic acid is provided which comprises in 3′→5′ direction:
the sequence complementary to the forward primer or the forward primer binding site,
the region complementary to the randomized region, as well as
the region of the second strand of the second adapter molecule which is partially complementary to the second constant sequence at the 3′ end of the nucleic acid of step (a) and/or (d),
(j) performing a nucleic acid synthesis, whereby the nucleic acid provided in (i) is reacted with a forward synthesis primer, modified nucleoside triphosphates, preferably 2′-fluoro nucleoside triphosphates, and a reverse transcriptase, whereby the primer hybridizes to the complementary sequence of the first nucleic acid strand of the first adapter molecule, and the primer consists of RNA or of a combination of DNA and RNA, whereby the primer consisting of a combination of DNA and RNA has at least a ribonucleotide at its 3′ end, in order to obtain a synthesis product,
(k) cleaving off the reverse transcription product from the synthesis product of step (j) and of the forward synthesis primer sequence of the synthesis product of step (j), in order to obtain a nucleic acid which is essentially identical to the nucleic acid of step (a) or (d),
(l) optionally repeating steps (a) to (k), whereby the nucleic acid of step (a) forms the heterogeneous population or is contained therein, and
(m) optionally sequencing the nucleic acid obtained in step (h).

61. The method according to claim 60, characterized in that the phosphorylating in step (fa) occurs by performing a kinase reaction.

62. The method according to claims 60 or 61, characterized in that the cleavage site is provided by a restriction enzyme cleavage site and the cleavage occurs by a restriction enzyme.

63. The method according to claims 60 or 61, characterized in that the cleavage site is provided by a ribonucleotide and the cleavage occurs via alkaline cleavage or via RNases.

64. The method according to any of claims 60 to 63, characterized in that the cleavage in accordance with step (l) occurs in an enzymatic manner, preferably by DNase, and/or that the forward synthesis primer sequence is removed by an RNase.

65. The method according to any of claims 60 to 64, characterized in that the nucleic acid of step (a) is a single-stranded nucleic acid consisting of modified nucleoside phosphates, in particular 2′-fluoro-modified nucleoside phosphates.

66. A method for the selection of a target molecule binding nucleic acid, in particular of aptamers, comprising the steps of
(a) providing a heterogeneous population of nucleic acids, in particular D-nucleic acids, whereby any of the nucleic acids comprises a region with a randomized sequence and a first constant sequence at the 5′ end and a second constant sequence at the 3′ end, and whereby the nucleic acids forming the population differ in the randomized sequence, whereby the first constant sequence comprises a forward primer sequence and the second constant sequence comprises a reverse primer binding site,
(b) contacting the population of nucleic acids with the target molecule,
(c) separating the nucleic acids not interacting with the target molecule,
(d) separating from the target molecule the nucleic acid(s) interacting with the target molecule,
(e) optionally repeating steps (a) to (d), whereby the nucleic acid(s) of step (d) form the heterogeneous population or are contained therein,
(f) second strand synthesis of a second strand complementary to the nucleic acid of step (a) and/or (d) and amplifying the second strand as well as the nucleic acids corresponding to the nucleic acid of step (a) and/or (d) by adding a reverse primer and a forward primer, whereby the reverse primer comprises a first and a second partial region, whereby the first partial region binds to the reverse primer binding site and the second partial region is arranged at the 5′ end of the reverse primer and comprises a promoter sequence for an RNA polymerase, whereby the synthesis product obtained by the second strand synthesis corresponds to the nucleic acid of step (a) and/or (d) and additionally has a sequence at its 3′ end which is complementary to the second partial region of the reverse primer,
(g) transcription of the synthesis product of step (f), whereby the transcription occurs upon addition of nucleoside phosphates and RNA polymerase and whereby the transcription product is subjected to a DNA digestion in order to obtain a transcription product which comprises in 3′→5′ direction the forward primer binding site, a region complementary to the randomized region of the nucleic acid of step (a), as well as the first partial region of the reverse primer,
(h) synthesis of a nucleic acid starting from the truncated transcription product of step (g), whereby the truncated transcription product is reacted with a forward synthesis primer, dNTPs and the reverse transcriptase, whereby the forward synthesis primer consists of deoxyribonucleotides,
(i) alkaline digestion of the reaction of step (h) for digesting the transcription product, in order to obtain a nucleic acid which is essentially identical to the nucleic acid of step (a) and (d),
(j) optionally repeating steps (a) to (i), whereby the nucleic acid(s) of step (i) forms the heterogeneous population or is contained therein, and
(k) optionally sequencing the nucleic acid(s) obtained in step (f) or (d).

67. The method according to claim 66, characterized in that the nucleic acid of step (a) is a deoxyribonucleic acid.

68. The method according to claims 66 or 67, characterized in that the promoter sequence is selected from the group comprising the promoter sequences of T7-RNA polymerase, T3-RNA polymerase and SP6 polymerase.

69. The method according to any of claims 23 to 68, characterized in that the selected nucleic acid(s) is/are selected from the group comprising aptamers, ribozymes, aptazymes, antisense molecules and siRNA.
